(19) United States
(12) Patent Application Publication
(10) Pub. No.: US 2021/0093724 A1
(43) Pub. Date: Apr. 1, 2021

(54) TOPICAL ANTI-ACNE COMPOSITION

(71) Applicant: Concept Matrix Solutions, Woodland Hills, CA (US)

(72) Inventors: Tony LaRosa, Woodland Hills, CA (US); Robert Davidson, Woodland Hills, CA (US); David Reid, Woodland Hills, CA (US)

(21) Appl. No.: 16/946,996

(22) Filed: Jul. 14, 2020

Related U.S. Application Data
(60) Provisional application No. 62/907,777, filed on Sep. 30, 2019.
(60) Provisional application No. 62/978,369, filed on Feb. 19, 2020.

(51) Int. Cl. A61K 47/46; A61K 47/22; A61K 9/06; A61K 47/10; A61K 47/24; A61K 47/44; A61K 9/00; A61K 47/34; A61K 31/60; A61K 31/327; A61K 47/14; A61K 47/02

(52) U.S. Cl. CPC A61K 47/46; A61K 47/22; A61K 9/06; A61K 47/10; A61K 47/24; A61K 47/02; A61K 9/0014; A61K 47/34; A61K 31/60; A61K 31/327; A61K 47/14; A61K 47/44

(57) ABSTRACT

Provided herein is a topical anti-acne composition that includes an external anti-acne agent, one or more pharmaceutically acceptable excipients, and at least one of a cannabinoid, terpene, and flavonoid. Also provided is a method that includes topically administering to a skin surface of a subject (e.g., human) the topical anti-acne composition.

### RELATED U.S. APPLICATION DATA

This application claims priority to provisional patent application No. 62/907,777 filed on Sep. 30, 2019, and provisional patent application No. 62/978,369 filed Feb. 19, 2020, the contents of which are incorporated by reference herein in their entirety.

### SUMMARY OF THE INVENTION

The present invention provides for a topical anti-acne composition that includes an external anti-acne agent; solvent; emulsifier; thickening agent; emollient; and at least one of a cannabinoid, terpene, and flavonoid.

The present invention also provides for a method that includes topically administering to a skin surface of a subject (e.g., human adolescent) the topical composition described herein.

The present invention also provides for a method that includes topically administering to a skin surface of a subject (e.g., human adolescent) the topical composition described herein to treat, prevent, ameliorate, or manage at least one of: (i) acne, (ii) inflammatory acne, (iii) acne causing bacteria (e.g., acne vulgaris), (iv) rosacea, (v) inflammation, (vi) clogged pores, (vii) dry sebum, (viii) inflammatory lesions (blemishes), (ix) pimples, (x) blackheads, and (xi) whiteheads.

The present invention also provides for a method that includes topically administering to a skin surface of a subject (e.g., human adolescent) the topical composition described herein to prevent & kill acne bacteria, reduce the number and/or severity of acne pimples, clear acne blemishes, help prevent development of new acne blemishes, cleanse the skin, remove excess oil, reduce the size and redness of acne breakout, renew skin, normalize skin, exfoliate the skin, provide smooth & soft skin texture, invigorate skin by lifting away dead skin cells, clear skin, reduce appearance of pores, help balance skin's pH level, provide clearer complexion, and/or provide softer & smoother skin.

### DETAILED DESCRIPTION OF THE INVENTION

Compositions of the present invention may be beneficial in preventing, treating, managing, and/or ameliorating a variety of dermal diseases or disorders, such as acne. Specifically, the compositions may be beneficial in preventing, treating, managing, and/or ameliorating at least one of: (i) acne, (ii) inflammatory acne, (iii) acne causing bacteria (e.g., acne vulgaris), (iv) rosacea, (v) inflammation, (vi) clogged pores, (vii) dry sebum, (viii) inflammatory lesions (blemishes), (ix) pimples, (x) blackheads, and (xi) whiteheads. More specifically, the compositions may be beneficial to prevent & kill acne bacteria, reduce the number and/or severity of acne pimples, clear acne blemishes, helps prevent development of new acne blemishes, cleanse the skin, remove excess oil, reduce the size and redness of acne breakout, renew skin, normalize skin, exfoliate the skin, provide smooth & soft skin texture, invigorate skin by lifting away dead skin cells, clear skin, reduce appearance of pores, help balance skin's pH level, provide clearer complexion, and/or provide softer & smoother skin.

In another aspect, the invention relates to a method of preventing, treating or ameliorating dermal diseases or disorders. The method includes administering to a person suffering from at least one of said diseases or disorders (or is at risk thereof), an effective amount of one or more compositions as described herein.

The compositions of the present invention include at least one of a cannabinoid, terpene, and flavonoid. Without wishing to be limited to any particular theory, it is currently believed that the cannabinoid, terpene, and/or flavonoid provides stability to the composition, which serves to prevent phase separation of an aqueous and a lipid phase in the composition at elevated temperatures (e.g. temperatures of more than about 25° C.), which might promote improved or prolonged contact to the skin, resulting in the observed increased retention times of the active(s) in the dermis and epidermis.

Across multiple topical dosage forms (e.g., creams, gels, lotions, ointments, foams, etc.) the cannabinoid, terpene, and/or flavonoid can be present in an amount, such that it exhibits activity as an active ingredient for the intended purpose (e.g., acne). In doing so, the cannabinoid, terpene, and/or flavonoid may further have a synergistic effect with the active ingredient(s) present therein. In other aspects, the cannabinoid, terpene, and/or flavonoid can be present in sub-therapeutic amounts.

It is currently believed that the topical use of the cannabinoid, terpene, and/or flavonoid provides additional benefits, which include: smoothing skin, strengthening underlying epidermal tissue, removing dead skin cells, balancing oil production, and helping the skin retain moisture levels. It is further believed that the cannabinoid, terpene, and/or flavonoid helps cleanse and moisturize the skin. Healthy skin is just like any other organ in your body: It continuously needs oxygen and nutrients to be brought to the cells, and toxins need to be washed away. It is further believed that the cannabinoid, terpene, and/or flavonoid contributes to the optimal skin health while leaving skin feeling and looking youthful.

It is also currently believed that some consumers have a preference to use a topical skin product that is environmentally friendly to produce and includes fewer toxic chemicals that are otherwise put into the environment to create topical skin products. Inclusion of substances, such as cannabinoid, terpene, and flavonoid (which are natural products), are viable options for such consumers.

Definitions:

The term “topical anti-acne composition” refers to a topical composition containing an external anti-acne agent, various inactive ingredients or excipients, and at least one of a terpene and cannabinoid. The composition is suitable for the prophylactic or therapeutic topical treatment of acne.

The term “acne” includes acne vulgaris and other dermal disorders such as preadolescent acne, acne rosacea (now known as rosacea), premenstrual acne, acne venenata, acne cosmetica, acne detergicans, acne cosmetica, acne excoriee, gram negative acne, steroid acne, acne conglobata, and nodulocystic acne.

The term “external anti-acne agent” or “anti-acne agent” refers to any chemical and/or biological agent (i.e. an antimicrobial peptide) that when topically administered at the site of acne, effectively treats and/or leads to a visible reduction of symptoms associated with the epithelial condition of acne vulgaris. Representative anti-acne agents include, for example adapalene, alcloxa, alkyl isoquinolinium bromide, aluminum chlorohydrex, aluminum hydroxide, azelaic acid, benzocaine, benzoic acid, benzoyl peroxide, boric acid, calcium polysulfide, calcium thiosulfate, camphor, cephalexin, chlorhydroxyquinoline (cloxyquin), chloroxylenol, cloxyquin (chlorhydroxyquinoline), clindamycin, coal tar, dapsone, dibenzothiophene, doxycycline, epidou, erythromycin, estrone, glycolic acid, hydrocortisone, isotretinoin, magnesium aluminum silicate, magnesium sulfate, metronidazole, minocycline, N-acetylcysteine, phenol, phenolate sodium, phenyl salicylate, picolinic acid, picolinic acid derivatives, picolinic acid analogs, povidone-iodine, pyrilamine maleate, pyruvic acid, resorcinol, resorcinol monoacetate, Retin-A, retinoids such as retinoic acid and its derivatives (e.g., cis and trans, esters), salicylic acid, sodium borate, sodium thiosulfate, spironolactone, sulfur, tazarotene, tetracaine hydrochloride, thymol, tretinoin, vitamin A, vitamin E, zinc oxide, zinc stearate, and zinc sulfide

The term “solvent” refers to a substance, typically a liquid at ambient conditions, capable of dissolving another substance (a solute), resulting in a solution. When one substance is dissolved into another, a solution is formed. This is opposed to the situation when the compounds are insoluble like sand in water. In solution, all of the ingredients are uniformly distributed at a molecular level and no residue remains. The mixing is referred to as miscibility, whereas the ability to dissolve one compound into another is known as solubility. However, in addition to mixing, both substances in the solution interact with each other. When something is dissolved, molecules of the solvent arrange themselves around molecules of the solute. Heat is involved and entropy is increased making the solution more thermodynamically stable than the solute alone. This arrangement is mediated by the respective chemical properties of the solvent and solute, such as hydrogen bonding, dipole moment and polarizability.

The term “emulsifier” refers to a substance capable of forming or promoting an emulsion. An emulsion is a mixture of two or more liquids that are normally immiscible (nonmixable or unblendable). Emulsions are part of a more general class of two-phase used systems of matter called colloids. Although the terms colloid and emulsion are sometimes interchangeably, emulsion should be used when both the dispersed and the continuous phase are liquids. In an emulsion, one liquid (the dispersed phase) is dispersed in the other (the continuous phase). Examples of emulsions include vinaigrettes, milk, mayonnaise, and some cutting fluids for metal working. The photo-sensitive side of photographic film is an example of a colloid.

The term “thickening agent” or “viscosity-increasing agent” refers to a substance which can increase the thickness or viscosity of a liquid without substantially changing its other properties. Some thickening agents are gelling agents (gellants), forming a gel, dissolving in the liquid phase as a colloid mixture that forms a weakly cohesive internal structure. Others act as mechanical thixotropic additives with discrete particles adhering or interlocking to resist strain. Typical gelling agents include natural gums, starches, pectins, agar-agar and gelatin. Often they are based on polysaccharides or proteins. Examples include: Alginic acid, sodium alginate, potassium alginate, ammonium alginate, calcium alginate—polysaccharides from brown algae; Agar (polysaccharide obtained from red algae); Carrageenan (polysaccharide obtained from red seaweeds); Locust bean gum (natural gum polysaccharide from the seeds of the carob tree); Pectin (polysaccharide obtained from apple or citrus-fruit); and Gelatin (made by partial hydrolysis of animal collagen). The thickening agent or viscosity-increasing agent also includes cellulose thickeners, such as e.g., hydroxypropyl cellulose, hydroxyethyl cellulose, hydroxypropyl cellulose hydroxypropyl methyl cellulose, methyl cellulose, with preferred products being hydroxypropyl cellulose), carbopols, polyacrylic acid, and polyvinyl alcohol.

The term “emollient” refers to lubricating ingredients (i.e., fats, phospholipids and sterols) that soften and smooth skin while helping it to retain moisture. Emollients are typically nonpolar and can come from natural or synthetic sources in the form of plant oils, mineral oils, shea butter, cocoa butter, petrolatum, cholesterol, silicones or animal oils (including emu, mink and lanolin).

The term “cannabinoid” refers to a class of diverse chemical compounds that act on cannabinoid receptors on cells that repress neurotransmitter release in the brain. These receptor proteins include the endocannabinoids (produced naturally in the body by humans and animals), the phytocannabinoids (found in Cannabis and some other plants), and synthetic cannabinoids (manufactured chemically). The most notable cannabinoid is the phytocannabinoid Δ9-tetrahydrocannabinol (THC), the primary psychoactive compound of Cannabis. Cannabidiol (CBD) is another major constituent of the plant, representing up to 40% in extracts of the plant resin. There are at least 85 different cannabinoids isolated from Cannabis, exhibiting varied effects. The cannabinoid can be synthetically prepared, or alternatively, can be obtained naturally (e.g., from plant matter). Either way, the cannabinoid can have the requisite purity (e.g., at least 95 wt. % pure, at least 98 wt. % pure, at least 99 wt. % pure, or at least 99.5 wt. % pure).

Cannabinoids isolated from Cannabis

| | |
|---|---|
| 1. | Cannabigerol ((E)-CBG-C5) |
| 2. | Cannabigerol monomethyl ether ((E)-CBGM-C5 A) |
| 3. | Cannabinerolic acid A ((Z)-CBGA-C5 A) |
| 4. | Cannabigerovarin ((E)-CBGV-C3) |
| 5. | Cannabigerolic acid A ((E)-CBGA-C5 A) |
| 6. | Cannabigerolic acid A monomethyl ether ((E)-CBGAM-C5 A) |
| 7. | Cannabigerovarinic acid A ((E)-CBGVA-C3 A) |
| 8. | (±)-Cannabichromene (CBC-C5) |
| 9. | (+)-Cannabichromenic acid ACBCA-C5 A |
| 10. | (+)-Cannabivarichromene or (+)-Cannabichromevarin (CBCV-C3) |
| 11. | (+)-Cannabichromevarinic acid A (CBCVA-C3 A) |
| 12. | (−)-Cannabidiol (CBD-C5) |
| 13. | Cannabidiol momomethyl ether (CBDM-C5) |
| 14. | Cannabidiol-C4 (CBD-C4) |
| 15. | (−)-Cannabidivarin CBDV-C3 |
| 16. | Cannabidiorcol (CBD-C1) |
| 17. | Cannabidiolic acid (CBDA-C5) |
| 18. | Cannabidivarinic acid (CBDVA-C3) |
| 19. | Cannabinodiol (CBND-C5) |
| 20. | Cannabinodivarin (CBND-C3) |
| 21 | Δ9-Tetrahydrocannabinol (Δ9-THC-C5) |
| 22. | Δ9-Tetrahydrocannabinol-C4 (Δ9-THC-C4) |
| 23. | Δ9-Tetrahydrocannabivarin (Δ9-THCV-C3) |
| 24. | Δ9-Tetrahydrocannabiorcol (Δ9-THCO-C1) |
| 25. | Δ9-Tetrahydro- cannabinolic acid A (Δ9-THCA-C5 A) |
| 26. | Δ9-Tetrahydro- cannabinolic acid B (Δ9-THCA-C5 B) |
| 27. | Δ9-Tetrahydro- cannabinolic acid-C4A and/or B (Δ9-THCA-C4A |
| | and/or B) |
| 28. | Δ9-Tetrahydro- cannabivarinic acid A (Δ9-THCVA-C3A) |
| 29. | Δ9-Tetrahydro- cannabiorcolic acid A and/or B (Δ9-THCOA-C1A |
| | and/or B) |
| 30. | (−)-Δ8-trans-(6aR,10aR)-A8-Tetrahydrocannabinol (Δ8-THC-C5) |
| 31. | (−)-Δ8-trans-(6aR,10aR)- Tetrahydrocannabinolic acid A (Δ8- |
| | THCA-C5 A) |
| 32. | (−)-(6aS,10aR)-Δ9-Tetrahydrocannabinol ((−)-cis-Δ9-THC-C5) |
| 33. | Cannabinol (CBN-C5) |
| 34. | Cannabinol-C4 (CBN-C4) |
| 35. | Cannabivarin (CBN-C3) |
| 36. | Cannabinol-C2 (CBN-C2) |
| 37. | Cannabiorcol (CBN-C1) |
| 38. | Cannabinolic acid A (CBNA-C5 A) |
| 39. | Cannabinol methyl ether (CBNM-C5) |
| 40. | (−)-(9R,10R)-trans- Cannabitriol ((−)-trans-CBT-C5) |
| 41. | (+)-(9S,10S)-Cannabitriol ((+)-trans-CBT-C5) |
| 42. | (±)-(9R,10S/9S,10R)- Cannabitriol ((±)-cis-CBT-C5) |
| 43. | (−)-(9R,10R)-trans-10-O-Ethyl-cannabitriol ((−)-trans-CBT-OEt-C5) |
| 44. | (±)-(9R,10R/9S,10S)- Cannabitriol-C3 ((±)-trans-CBT-C3) |
| 45. | 8,9-Dihydroxy-Δ6a(10a)- tetrahydrocannabinol (8,9-Di-OH-CBT-C5) |
| 46. | Cannabidiolic acid A cannabitriol ester (CBDA-C5 9-OH-CBT-C5 |
| | ester) |
| 47. | (−)-(6aR,9S,10S,10aR)- 9,10-Dihydroxy- hexahydrocannabinol, |
| | Cannabiripsol (Cannabiripsol-C5) |
| 48. | (−)-6a,7,10a-Trihydroxy-Δ9-tetrahydrocannabinol ((−)-Cannabitetrol) |
| 49. | 10-Oxo-Δ6a(10a)- tetrahydrocannabinol (OTHC) |
| 50. | (5aS,6S,9R,9aR)- Cannabielsoin (CBE-C5) |
| 51. | (5aS,6S,9R,9aR)- C3-Cannabielsoin (CBE-C3) |
| 52. | (5aS,6S,9R,9aR)- Cannabielsoic acid A (CBEA-C5 A) |
| 53. | (5aS,6S,9R,9aR)- Cannabielsoic acid B (CBEA-C5 B) |
| 54. | (5aS,6S,9R,9aR)- C3-Cannabielsoic acid B (CBEA-C3 B) |
| 55. | Cannabiglendol-C3 (OH-iso-HHCV-C3) |
| 56. | Dehydrocannabifuran (DCBF-C5) |
| 57. | Cannabifuran (CBF-C5) |
| 58. | (−)-Δ7-trans-(1R,3R,6R)-Isotetrahydrocannabinol |
| 59. | (±)-Δ7-1,2-cis- (1R,3R,6S/1S,3S,6R)- Isotetrahydro- cannabivarin |
| 60. | (−)-Δ7-trans-(1R,3R,6R)- Isotetrahydrocannabivarin |
| 61. | (±)-(1aS,3aR,8bR,8cR)- Cannabicyclol (CBL-C5) |
| 62. | (±)-(1aS,3aR,8bR,8cR)- Cannabicyclolic acid A (CBLA-C5 A) |
| 63. | (±)-(1aS,3aR,8bR,8cR)- Cannabicyclovarin (CBLV-C3) |
| 64. | Cannabicitran (CBT-C5) |
| 65. | Cannabichromanone (CBCN-C5) |
| 66. | Cannabichromanone-C3 (CBCN-C3) |
| 67. | Cannabicoumaronone (CBCON-C5) |
| 68. | Cannabielsoin acid A (CBEA-A) |
| 69. | 10-Ethoxy-9-hydroxy-delta-6a-tetrahydrocannabinol |
| 70. | Cannabitriolvarin (CBTV) |
| 71. | Delta-9-tetrahydrocannabiorcolic acid (THCA-C1) |
| 72. | Delta-7-cis-iso-tetrahydrocanna |
| 73. | Cannabichromanon (CBCF) |

Structure of common cannaboinoids

| | | |
|---|---|---|
| 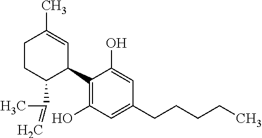 [SMILES: C=C(C)[C@@H]1CCC(C)=C[C@H]1c1c(O)cc(CCCCC)cc1O] | 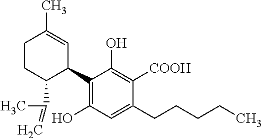 [SMILES: C=C(C)[C@@H]1CCC(C)=C[C@H]1c1c(O)cc(CCCCC)c(C(=O)O)c1O] | 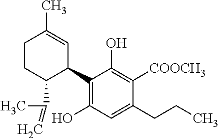 [SMILES: C=C(C)[C@@H]1CCC(C)=C[C@H]1c1c(O)cc(CCC)c(C(=O)OC)c1O] |
| CBD | CBDA | CBDVA-OMe |
| 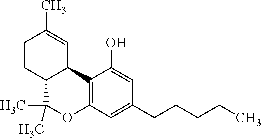 [SMILES: CCCCCc1cc(O)c2c(c1)OC(C)(C)[C@@H]1CCC(C)=C[C@@H]21] | 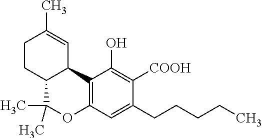 [SMILES: CCCCCc1cc2c(c(O)c1C(=O)O)[C@@H]1C=C(C)CC[C@H]1C(C)(C)O2] | 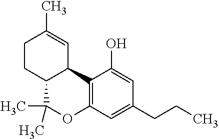 [SMILES: CCCc1cc(O)c2c(c1)OC(C)(C)[C@@H]1CCC(C)=C[C@@H]21] |
| THC | THCA | THCV |
| 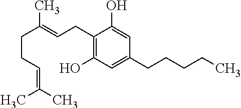 [SMILES: CCCCCc1cc(O)c(C/C=C(\C)CCC=C(C)C)c(O)c1] | 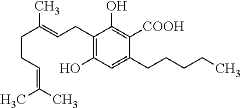 [SMILES: CCCCCc1cc(O)c(C/C=C(\C)CCC=C(C)C)c(O)c1C(=O)O] | 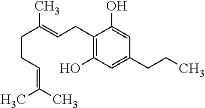 [SMILES: CCCc1cc(O)c(C/C=C(\C)CCC=C(C)C)c(O)c1] |
| CBG | CBGA | CBGV |
| 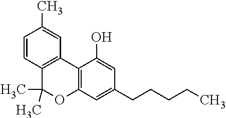 [SMILES: CCCCCc1cc(O)c2c(c1)OC(C)(C)c1ccc(C)cc1-2] | 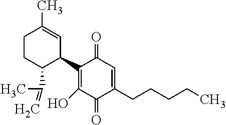 [SMILES: C=C(C)[C@@H]1CCC(C)=C[C@H]1C1=C(O)C(=O)C(CCCCC)=CC1=O] | 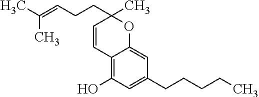 [SMILES: CCCCCc1cc(O)c2c(c1)OC(C)(CCC=C(C)C)C=C2] |
| CBN | CBQ | CBC |
| 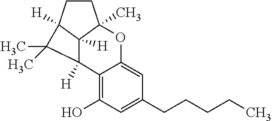 | 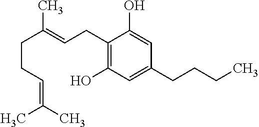 [SMILES: CCCCc1cc(O)c(C/C=C(\C)CCC=C(C)C)c(O)c1] | 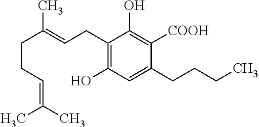 [SMILES: CCCCc1cc(O)c(C/C=C(\C)CCC=C(C)C)c(O)c1C(=O)O] |
| CBL | CBGB | CBGBA |
| 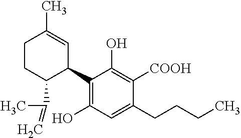 [SMILES: C=C(C)[C@@H]1CCC(C)=C[C@H]1c1c(O)cc(CCCC)c(C(=O)O)c1O] | 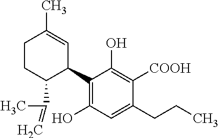 [SMILES: C=C(C)[C@@H]1CCC(C)=C[C@H]1c1c(O)cc(CCC)c(C(=O)O)c1O] | 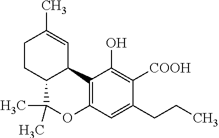 [SMILES: CCCc1cc2c(c(O)c1C(=O)O)[C@@H]1C=C(C)CC[C@H]1C(C)(C)O2] |
| CBDBA | CBDVA | THCVA |

Synthetically prepared cannabinoids, that are commercially available (e.g., Purisys™ of Athens, Ga.), are provided below.

| | | | |
|---|---|---|---|
| | | Alkyl | |
| | | Tail | |
| Common Name | Other Names | Length | CAS # |

TETRAHYDROCANNABIVARIN FAMILY

| | | | |
|---|---|---|---|
| Δ9-Tetrahydrocannabivarin | Δ9-THCV | C3 | 31262-37-0 |
| Δ8-Tetrahydrocannabivarin | Δ8-THCV | C3 | 31262-38-1 |
| Δ9-Tetrahydrocannabivarin | Δ9-THCV-NE | C3 | N/A |
| Naphtoylester | | | |
| Δ8-Tetrahydrocannabivarin | Δ8-THCV-NE | C3 | N/A |
| Naphtoylester | | | |
| Δ9-Tetrahydrocannabivarinic | Δ9-THCVA-A, | C3 | 39986-26-0 |
| Acid | Δ9-THC- VA- | | |
| | B | | |

CANNABIDIOLVARIN FAMILY

| | | | |
|---|---|---|---|
| (−)-Cannabidivarin | (−)-CBDV | C3 | 24274-48-4 |
| (+)-Cannabidivarin | (+)-CBDV | C3 | 1637328-94-9 |
| Cannabidivarinic Acid | CBDVA | C3 | 31932-13-S |
| Cannabidivarin Quinone | CBQV | C3 | N/A |

TETRAHYDROCANNABIBUTOL FAMILY

| | | | |
|---|---|---|---|
| Δ9-Tetrahydrocannabibutol | Δ9-THCB | C4 | 60008-00-6 |
| Δ8-Tetrahydrocannabibutol | Δ8-THCB | C4 | 51768-59-3 |
| Δ9-Tetrahydrocannabibutol | Δ9-THCB-NE | C4 | 60007-98-9 |
| Naphtoylester | | | |
| Δ8-Tetrahydrocannabibutol | Δ8-THCB-NE | C4 | N/A |
| Naphtoylester | | | |
| Δ9-Tetrahydrocannabibutolic | Δ9-THCBA-A, | C4 | 60007-98-9 |
| Acid | Δ9-THC- BA- | | |
| | B | | |

CANNABIDIBUTOL FAMILY

| | | | |
|---|---|---|---|
| (−)-Cannabidibutol | (−)-CBDB | C4 | 60113-11-3 |
| (+)-Cannabidibutol | (+)-CBDB | C4 | N/A |
| Cannabidibutolic Acid | CBDBA | C4 | N/A |

RARE CANNABINOIDS

| | | | |
|---|---|---|---|
| Cannabinol | CBN, USP | C5 | 521-35-7 |
| | Impurity | | |
| Cannabinolic Acid | CBNA | C5 | 2808-39-1 |
| Cannabigerol | CBG | C5 | 25654-31-3 |
| Cannabigerolic Acid | CBGA | C5 | 25555-57-1 |
| Cannabichromene | CBC | C5 | 20675-51-8 |
| Cannabichromenic Acid | CBCA | C5 | 185505-15-1 |
| Cannbicyclol | CBL | C5 | 21366-63-2 |
| Cannabicyclolic Acid | CBLA | C5 | 2283314-84-9 |
| Cannabivarin | CBNV | C3 | 33745-21-0 |
| Cannabivarinic Acid | CBNVA | C3 | 64846-02-2 |
| Cannbigerivarin | CBGV | C3 | 55824-11-8 |
| Cannabigerivarinic Acid | CBGVA | C3 | 64924-07-8 |
| Cannbichromevarin | CBCV | C3 | 57130-04-8 |
| Cannabichromevarinic Acid | CBCVA | C3 | 64898-02-8 |
| Cannabicyclolvarin | CBLV | C3 | 55870-47-8 |
| Cannabicyclolvarinic Acid | CBLVA | C3 | 2281847-63-8 |
| 3-Butylcannabinol | CBNB | C4 | 60007-99-0 |
| 3-Butylcannabinolic Acid | CBNBA | C4 | N/A |
| Cannabigerol Butyl | CBGB | C4 | N/A |
| Cannabigerol Butyric Acid | CBGBA | C4 | N/A |
| Cannabichromene Butyl | CBCB | C4 | N/A |
| Cannabichromene Buytric | CBCBA | C4 | N/A |
| Acid | | | |
| Cannabicyclol Butyl | CBLB | C4 | N/A |
| Cannabicyclol Butyric Acid | CBLBA | C4 | N/A |

The term “terpene” refers to a hydrocarbon or derivative thereof, found as a natural product and biosynthesized by oligomerization of isoprene units. A terpene can be acyclic, monocyclic, bicyclic, or multicyclic. Examples include, e.g., sesquiterpenes (e.g., (−)-β-caryophyllene, humulene, vetivazulene, guaiazulene, longifolene, copaene, and patchoulol), monoterpenes (e.g., limonene and pulegone), monoterpenoids (e.g., carvone), diterpenes (e.g., taxadiene), and triterpenes (e.g., squalene, betulin, betulinic acid, lupane, lupeol, betulin-3-caffeate, allobetulin, and cholesterol). The terpene can be synthetically prepared, or alternatively, can be obtained naturally (e.g., from plant matter). Either way, the terpene can have the requisite purity (e.g., at least 95 wt. % pure, at least 98 wt. % pure, at least 99 wt. % pure, or at least 99.5 wt. % pure).

| Terpene | Plant | Genus | Species |
|---|---|---|---|
| Myrcene | Myrtles | Myrtus | communis; nivellei; |
| | | | phyllireaefolia |
| | Cannabis | Cannabis | sativa; ruderalis; indica |
| Linalool | Mint | Mentha | spicata; arvensis; |
| | | | canadensis |
| | Lavender | Lavandula (subgenus: | spica; angustifolia; |
| | | Fabricia; Sabaudia) | latifolia; lanata; dentata; |
| | | | stoechas; pedunculata; |
| | | | viridis |
| Terpineol | Orange peel | Citrus | reticulata |
| | Junipers | Juniperus | communis; chinensis; |
| | | | conferta; rigida |
| Camphene | Chrysanthemum | Chrysanthemum | indicum |
| | Ginger | Zingiber | officinale |
| Bisabolol | Chamomile | Matricaria (or | chamomilla (or nobile) |
| | | Chamaemelum) | |
| | Figwort | Myoporum | crassifolium |
| Nerolidol | Cannabis | Cannabis | sativa; ruderalis; indica |
| Limonene | Citrus Lemon | Citrus | limon |
| Humulene | Hops | Humulus | lupulus; japonicus; |
| | | | yunnanensis |
| Terpinolene | Cannabis | Cannabis | sativa; ruderalis; indica |
| Carene | Rosemary | Salvia | rosmarinus; jordanii |
| | Cedar | Cedrus | atlantica; brevifolia; |
| | | | deodara; libani |
| Eucalyptol | Eucalyptus | Eucalyptus | obliqua |
| | Cannabis | Cannabis | sativa; ruderalis; indica |
| | Camphor laurel | Cinnamomum | camphora |
| | Bay leaves | Laurus | nobilis |
| | Wormwood | Artemisia | vulgaris |
| Ocimene | Hops | Humulus | lupulus; japonicus; |
| | | | yunnanensis |
| | Kumquats | Citrus | japonica |
| | Mango | Mangifera | indica |
| | Basil | Ocimum | basilicum |
| | bergamot orange | Citrus | x aurantium |
| Carophyllene | Peppercorn | Piper | nigrum |
| | Cloves | Syzgium | aromaticum |
| | Cannabis | Cannabis | sativa; ruderalis; indica |
| | Rosemary | Salvia | rosmarinus; jordanii |
| | Hops | Humulus | lupulus; japonicus; |
| | | | yunnanensis |
| Valencene | Nootka cypress | Callitropsis | nootkatensis |
| Geraniol | Roses | Rosa (subgenus: | persica; minutifolia; |
| | | Banksianae, Bracteatae, | stellata |
| | | Caninae, Carolinae, | |
| | | Chinensis, Gallicanae, | |
| | | Gymnocarpae, | |
| | | Laevigatae, | |
| | | Pimpinellifoliae, | |
| | | Synstylae) | |
| | Wine grapes | Vitis | vinifera |
| Borneol | Borneo camphor | Dryobalanops | aromatica |
| | Ngai camphor; | Blumea | balsamifera |
| | sambong | | |
| Pulegone | Catnip | Nepeta | cataria |
| | Peppermint | Mentha | piperita |
| | Pennyroyal | Hedeoma | pulegioides |
| Guaiazulene | Chamomile | Matricaria (or | chamomilla (or nobile) |
| | | Chamaemelum) | |
| | Guaiacum tree | Guaiacum | sanctum, angustifolium, |
| | | | coulteri, officinale |
| Lupeol | Lupine seed | Lupinus | luteus |
| Lupane | Lupine seed | Lupinus | luteus |
| Betulin | Brich tree | Betula (Subgenus: | alleghaniensis, cordifolia, |
| Betulinic acid | | Betulenta, Betulaster, | glandulosa, lenta, |
| Lupeol | | Neurobetula, | michauxii, minor, nana, |
| | | Chamaebetula) | neoalaskana, nigra, |
| | | | occidentalis, papyrifera, |
| | | | populifolia, pumila, uber |
| Squalene | Amaranth seed | Amaranthus (subgenus: | acanthochiton, |
| | | Acnida; Albersia) | acutilobus, albus, |
| | | | anderssonii, californicus |
| | Wheat germ | Triticum | aestivum |
| | Olive | Olea | europaea |
| Carvone | Caraway seed | Carum | carvi |
| | Spearmint | Mentha | spicata |
| | Dill | Anethum | graveolens |
| Patchoulol | Patchouli | Pogostemon | cablin |
| Copaene | Copaiba tree | Copaifera | langsdorfii |
| Longifolene | Pine | Pinus | longifolia |
| Pinene | Pine | Pinus (subgenus: | densata, densiflora, |
| | | Strobus; Pinus) | pinea, sylvestris |
| Vetivazulene | Vetiver | Chrysopogon | zizanioides |
| Nerol | Lemon Grass | Cymbopogon | nardus; citratus; |
| | | | flexuosus; martinii; |
| | | | schoenanthus |

Synthetically prepared terpenes, which are commercially available (e.g., Purisys™ of Athens, Ga.), are provided below.

| Terpene | CAS# |
|---|---|
| Alpha-Pinene | 51634232009 |
| Beta-Pinene | 51634232109 |
| Beta-Myrcene | 51634232209 |
| Alpha-Terpinene | 51634232309 |
| Limonene | 51634232409 |
| Beta-Ocimene | 51634232509 |
| Terpinolene | 51634232609 |
| Linalool | 51634232709 |
| Fenchyl Alcohol | 51634232809 |
| Borneol Isomers | 51634232909 |
| Alpha-Terpineol | 51634233009 |
| Trans-caryophyllene | 51634233109 |
| Alpha-humulene | 51634233209 |
| Trans-nerolidol | 51634233309 |
| Guaiol | 51634233409 |
| Alpha-Bisabolol | 51634233509 |

The term “flavonoid” refers to ubiquitous plant natural products with various polyphenolic structures. Flavonoids can be extracted from fruits, vegetables, grains, bark, roots, stems, flowers, and teas or can be biosynthetically produced. The role of flavonoids in plants includes UV protection, aid in plant growth, defense against plaques, and provide the color and aroma of flowers.

Flavonoids can be divided into classes (e.g., anthocyanin, chalcone, flavone, flavonol, isoflavone, and flavonone) and subclasses depending on the carbon of the C ring on which the B ring is attached and the degree of unsaturation and oxidation of the C ring.

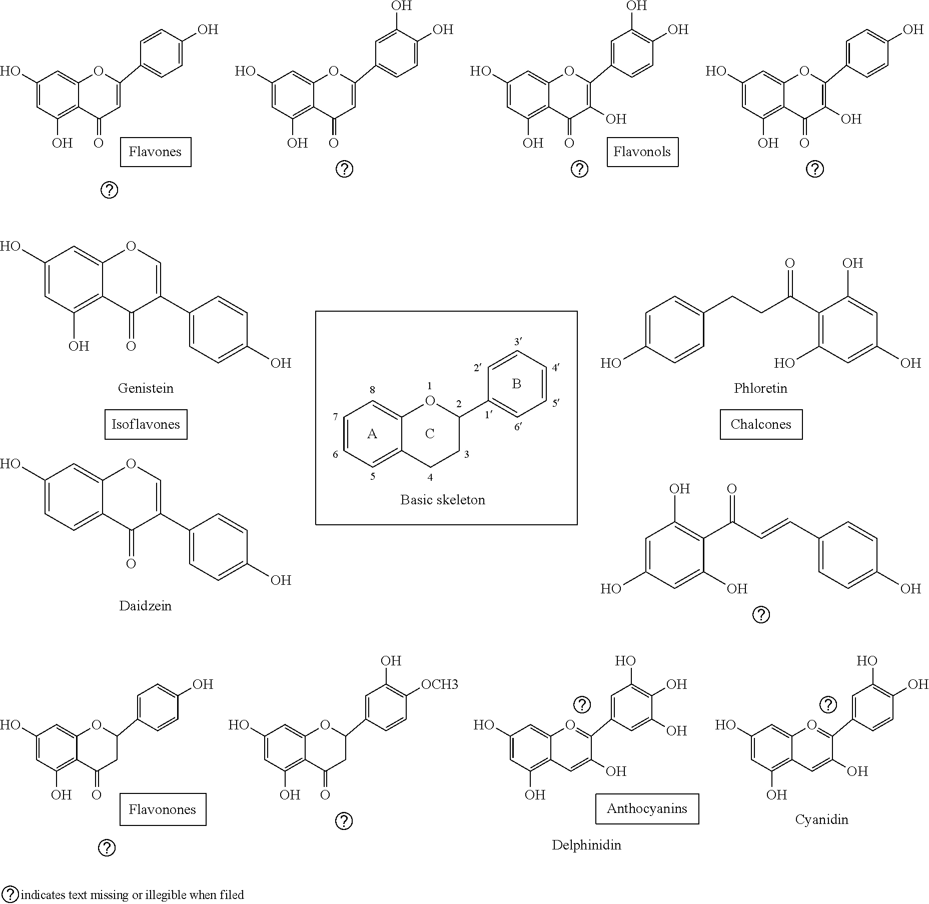

| | | | Examples of |
|---|---|---|---|
| Flavonoid classes | Subclasses | Natural sources | natural sources |
| Anthocyanins | Cyanidin, Malvidin, | Fruits, vegetables, | Cranberries, plums, |
| | Delphinidin, Peonidin | nuts, dried fruits, | cherries, sweet |
| | | medicinal plants | potatoes, black |
| | | | currants, red |
| | | | grapes, merlot |
| | | | grapes, raspberries, |
| | | | strawberries, |
| | | | blueberries, |
| | | | bilberries and |
| | | | blackberries |
| Chalcones | Phloretin, Arbutin, | Fruits, vegetables, | Tomatoes, pears, |
| | Phlioridzin | medicinal plants | strawberries, |
| | | | bearberries and |
| | | | certain wheat |
| | | | products |
| Flavonones | Hesperitin, Naringin, | Fruits (citrus), | Oranges, lemons, |
| | Naringenin, Eriodictyol, | medicinal plants | grapes, rosehips |
| | Hesperidin | | |
| Flavones | Apigenin, Tangeretin, | Fruits, medicinal | Celery, parsley, red |
| | Baicalein, Rpoifolin | plants | peppers, |
| | | | chamomile, mint, |
| | | | ginkgo biloba, |
| | | | broccoli, green |
| | | | pepper, thyme, |
| | | | dandelion, perilla, |
| | | | tea, carrot, |
| | | | rosemary, oregano, |
| | | | Cannabis sativa |
| Flavonols | Quercetin, Myricetin, | Fruits, vegetables, | Onion, kale, |
| | Rutin, Morin, | medicine plants | lettuce, tomatoes, |
| | Kaempferol | | apples, grapes, |
| | | | berries, tea, red |
| | | | wine, broccoli, |
| | | | potatoes, brussel |
| | | | sprouts, squash, |
| | | | cucumbers, lettuce, |
| | | | green beans, |
| | | | spinach, peaches, |
| | | | blackberries |
| Isoflavonoids | Genistin, Genistein, | Legumes, | Soybeans, lupin, |
| | Daidzein, Glycetein, | medicinal plants | fava beans, kudzu, |
| | Daidzin | | psoralea, red |
| | | | clover, alfalfa |
| | | | sprouts, peanuts, |
| | | | chickpeas |

| Flavonoid classes | Structure of flavonoid classes |
|---|---|
| Anthocyanins | Double bonds between positions 1 and 2, 3 and 4 |
| | of the C ring; Hydroxyl groups at positions 5 |
| | and 7 in the A ring and 3′, 4′ and/or 5′ of the |
| | B ring; Methylation or acylation at the hydroxyl |
| | groups on the A and B rings vary |
| Chalcones | Absence of ‘C ring’ of the basic flavonoid skeleton |
| | structure |
| Flavonones | C ring is saturated (contains no double bonds) |
| Flavones | Double bond between positions 2 and 3 and a ketone |
| | in position 4 of the C ring; Most have a hydroxyl |
| | group in position 5 or 7 of the A ring of the A |
| | ring or 3′ and 4′ of the B ring (varies according |
| | to the taxonomic classification of the particular plant) |
| Flavonols | Double bond between positions 2 and 3, a ketone |
| | in position 4 and hydroxyl group in position 3 of |
| | the C ring, the ketone group the C ring may also |
| | be glycosylated; very diverse in methylation and |
| | hydroxylation patterns |
| Isoflavonoids | B ring is attached to the 3 position of the C ring |
| | and contains a hydroxyl group at the 4′ position; |
| | hydroxylation of the A ring varies |

Studies on flavonoids have revealed an increasing number of health benefits showing anti-oxidant, anti-inflammatory, anti-mutagenic, and anti-carcinogenic properties by inhibiting numerous pro-inflammatory and pro-oxidative enzymes (e.g., xanthine oxidase (XO), cyclo-oxygenase (COS), lipoxygenase, phosphoinositide 3-kinase, and acetylcholinesterase). This may have benefits towards numerous diseases and medical conditions (e.g., pain, cancer, arthersclerosis, Alzheimer's disease). There is a growing interest in the medicinal properties of Cannabis (Cannabis sativa, Cannabis indica, Cannabis ruderalis). Studies have shown that Cannflavin A and Cannflavin B, prenylated flavones, have anti-inflammatory properties greater than aspirin. Cannflavin A and B can be isolated from Cannabis sativa and biosynthesized.

Synthetically prepared flavonoids, which are commercially available (e.g., Cannflavin B from Toronto Research Chemicals), are provided below.

| Flavonoid | CAS# |
|---|---|
| Cannflavin A | 76735-57-4 |
| Cannflavin B | 76735-58-5 |
| Myricetin | 529-44-2 |
| (−)-Epigallocathechin | 989-51-5 |
| gallate | |
| Polyphenon 60 from green | 138988-88-2 |
| tea | |
| (−)-Gallocathechin | 3371-27-5 |
| Kaempferol | 520-18-3 |
| (±)-Catechin hydrate | 7295-85-4 (anhydrous) |
| Galangin | 548-83-4 |
| Hesperidin | 520-26-3 |
| Baicalein | 491-67-8 |
| Icariin | 489-32-7 |
| Orientin | 28608-75-5 |
| Liquiritigenin | 578-86-9 |
| Acacetin | 480-44-4 |
| Diosmetin | 520-34-3 |
| Scutellarein | 529-53-3 |
| Luteolin | 491-70-3 |

The term “transdermal delivery agent” refers to a substance that aids or facilitates the passage of desired compounds, such as pharmaceutically active ingredients (e.g., external anti-acne agent), cannabinoids, and/or terpenes, at least partially through one or more layers of the skin, including the dermis and epidermis.

The term “antioxidant” refers to an oxidation preventive agent that reduces excessive production of reactive oxygen species. Antioxidants can come from natural products or sources such as vitamin E (tocopherol), vitamin C (ascorbic acid), vitamin A, β-carotene, superoxide dismutase, coenzyme Q. Synthetically derived antioxidants include 3,5-tert-butyl-4-hydroxytoluene (BHT) and 2,3-tert-butyl-hydroxyanisole (BHA) which have been developed to inhibit oxidation of a lipid or the like.

The term “fragrance” refers to a combination of chemicals that gives each product its distinct scent. Chemical ingredients may be derived from synthetic or natural raw materials that can add or mask a scent. The ingredients are capable of imparting or modifying the odor of skin or hair or other substrate.

The term “coloring agent” refers to a natural or synthetic compound that provides a pigment used to enhance the product's appearance and aesthetic value.

The term “exfoliant” refers to an additive used in an attempt to remove dead skin cells exposing the living layer. This provides the skin a smoother softer more youthful appearance, as well as allowing the living layers to grow and stay healthy. Exfoliating topical compositions conventionally use an abrasive compound in a scrubbing motion to remove dead skin cells from the face. Common exfoliants include powders from ground apricot seeds, crushed walnut shells, coconut shells, almond seeds and shells, and sawdust, various solid polymer powders, and various inorganic particles such as sand, salt, alumina, silica, alumino-silicates, lava stone, various phosphates, borates, sulfates, and carbonates.

The term “skin surface” refers to interface of an organism with the environment, which prevents moisture loss from the body, and is a barrier functioning to prevent the invasion of biotoxic substances, such as microorganisms and allergens, from the environment. The skin contains two layers consisting of an outer epidermis and an inner dermis.

The term “human” refers to a person who can benefit from the pharmaceutical formulations and methods of the present invention. The person that could benefit from the presently described pharmaceutical formulations and methods may be an adolescent or adult. A human may be referred to as an individual, patient, subject, or recipient.

The term “topical formulation” is used herein to generally include a formulation that can be applied to skin or a mucosa. Topical formulations may, for example, be used to confer therapeutic benefit to a patient or cosmetic benefits to a consumer. Topical formulations can be used for both topical and transdermal administration of substances. The topical formulations can be configured and formulated to exist in various dosage forms, such as, e.g., gel, pump gel, gel packet, cream, lotion, roll-on liquid, roll-on gel, spray, pump spray, aerosol spray, stick, patch, ointment, liniment, or balm.

The term “topical administration” is used herein to generally include the delivery of a substance, such as a therapeutically active agent (e.g., external anti-acne agent), to the skin or a localized region of the body.

The term “transdermal administration” is used herein to generally include administration through at least a portion of the skin. Transdermal administration is often applied for delivering desired substances to tissues underlying the skin with minimal systemic absorption. As such, the transdermal administration delivers desired substances at least partially through one or more layers of the skin, including the dermis and epidermis.

The term “subject” is used herein to generally include humans, particularly human adolescents (e.g., 12-17 years old) and human adults (e.g., at least 18 years old).

The term “effective amount” is used herein to generally include an amount of topical formulation (or external anti-acne agent) effective for treating or preventing a disease in a subject as described herein.

The term “treating” with regard to a subject, refers to improving at least one symptom of the subject's disorder. Treating includes curing, improving, or at least partially ameliorating the disorder or the symptoms thereof.

### Method of Manufacturing

As described herein, the compositions of the present invention are suitable for topical (e.g., dermal or intradermal) administration and include, e.g., liquid or semi-solid preparations (e.g., liniments, lotions, gels, sprays, foams, film forming systems, microneedles, micro- or nano-emulsions), and oil-in-water or water-in-oil emulsions (e.g., creams, ointments or pastes). These topical dosage forms can be prepared employing well-known and routine procedures, equipment, techniques, and substances. See, e.g., A. Williams, Transdermal and Topical Delivery Systems, Pharmaceutical Press, London and Chicago, 2003; L. Lachman, The Theory and Practice of industrial Pharmacy, 4th Ed., Stipes Publishing, 2015; Remington, Pharmaceutical Sciences, 22^{nd} Rev., Pharmaceutical Press, 2012; H. Benson, Topical and Transdermal Drug Delivery: Principles and Practice 1st Edition, Wiley, 2012; D. Osborne, Topical Drug Delivery Formulations (Drugs and the Pharmaceutical Sciences) 1st Edition, CRC Press, 1989; and M. Brown, The Art and Science of Dermal Formulation Development (Drugs and the Pharmaceutical Sciences) 1st Edition, CRC Press, 2019.

### EXAMPLES

Production Examples 1-306 provided below illustrate active ingredients (external anti-acne agents) formulated with suitable inactive ingredients (excipients), to provide various dosage forms (e.g., creams and lotions). Production Example 307 provided below illustrates active ingredients (external anti-acne agents). As described herein, the formulations illustrated in Production Examples 1-306 can be formulated to further include a cannabinoid, terpene, flavonoid, or combination thereof. Additionally, the active ingredients (external anti-acne agents) illustrated in Production Example 307 can be formulated (1) with suitable inactive ingredients (excipients) and (2) with a cannabinoid, terpene, flavonoid, or combination thereof, to provide various dosage forms (e.g., creams and lotions).

### Production Example 1: Benzoyl Peroxide Cream

ACTIVE INGREDIENT: Benzoyl Peroxide 10%
INACTIVE INGREDIENTS: Water, Propylene Glycol, Aluminium Hydroxide, Bentonite, Glyceryl Stearate SE, PEG-12, Isopropyl Myristate, Simethicone, Methylparaben, Carbomer, Potassium Hydroxide, Propylparaben, Titanium Dioxide, Iron Oxides

### Production Example 2: Benzoyl Peroxide Cream

ACTIVE INGREDIENT: Benzoyl peroxide 10%
INACTIVE INGREDIENTS: Water, Propylene Glycol, Aluminium Hydroxide, Bentonite, Glyceryl Stearate SE, PEG-12, Isopropyl Myristate, Methylparaben, Carbomer, Potassium Hydroxide, Propylparaben.

### Production Example 3: Benzoyl Peroxide Cream

ACTIVE INGREDIENT: Benzoyl peroxide 10%
Inactive ingredients: Water, Gluconolactone, Glyceryl Stearate, PEG-40 Stearate, Glycerin, PEG-100 Stearate, Cetyl Alcohol, Dimethicone, Magnesium Aluminum Silicate, Xanthan Gum, Benzyl Alcohol, Ammonium Hydroxide, Arginine, Benzoic Acid, Sorbic Acid, Titanium Dioxide

### Production Example 4: Benzoyl Peroxide Cream

ACTIVE INGREDIENT: Benzoyl peroxide 10%
INACTIVE INGREDIENTS: Water, Propylene Glycol, Aluminum Hydroxide, Bentonite, Glyceryl Stearate SE, PEG-12, Isopropyl Myristate, Simethicone, Methylparaben, Carbomer, Potassium Hydroxide, Propylparaben, Titanium Dioxide, Iron Oxides

### Production Example 5: Benzoyl Peroxide Cream

ACTIVE INGREDIENT: Benzoyl peroxide 10%
INACTIVE INGREDIENTS: Water, Propylene Glycol, Aluminum Hydroxide, Bentonite, Glyceryl Stearate SE, PEG-12, Isopropyl Myristate, Methylparaben, Carbomer, Potassium Hydroxide, Propylparaben

### Production Example 6: Benzoyl Peroxide Cream

ACTIVE INGREDIENT: Benzoyl peroxide 10%
INACTIVE INGREDIENTS: water, gluconolactone, glyceryl stearate, glycerin. PEG-40 stearate, PEG-100 stearate, cetyl alcohol, dimethicone, magnesium aluminum silicate, xanthan gum, benzyl alcohol, ammonium hydroxide, arginine, benzoic acid, sorbic acid, titanium dioxide

### Production Example 7: Benzoyl Peroxide Cream

ACTIVE INGREDIENT: Benzoyl peroxide 10%
INACTIVE INGREDIENTS: Water, Gluconolactone, Glyceryl Stearate, Glycerin, PEG-40 Stearate, PEG-100 Stearate, Cetyl Alcohol, Dimethicone, Magnesium Aluminum Silicate, Xanthan Gum, Phenoxyethanol, Ammonium Hydroxide, Isopropylparaben, Butylparaben, Isobutylparaben, Arginine, Titanium Dioxide.

### Production Example 8: Benzoyl Peroxide Lotion

ACTIVE INGREDIENT: Benzoyl peroxide 2.5%
INACTIVE INGREDIENTS: Water, Carbomer Homopolymer type B, Ethylhexylglycerin, Sodium Hydroxide, Chlorphenesin, Disodium EDTA, Laureth-4, Hydroxypropyl Methylcellulose

### Production Example 9: Benzoyl Peroxide Cream

ACTIVE INGREDIENT: Benzoyl peroxide 5%
INACTIVE INGREDIENTS: Water, Carbomer, Benzyl Alcohol, Sodium Hydroxide, Laureth-4, Disodium EDTA, Hydroxypropyl Methylcellulose

### Production Example 10: Benzoyl Peroxide Gel

ACTIVE INGREDIENT: Benzoyl peroxide 3.5%
INACTIVE INGREDIENTS: Water, Glycerin, Kaolin, Bentonite, Sodium Methyl Cocoyl Taurate, Titanium Dioxide, Citric Acid, Xanthan Gum, Benzyl Alcohol, Sodium Citrate, Sodium Hydroxide, Fragrance, Disodium EDTA, Menthol

### Production Example 11: Benzoyl Peroxide Suspension

ACTIVE INGREDIENT: Benzoyl peroxide 3.7%
INACTIVE INGREDIENTS: Water, Glycerin, Kaolin, Bentonite, Sodium Methyl Cocoyl Taurate, Titanium Dioxide, Polyethylene, Xanthan Gum, Citric Acid, Sodium Citrate, Trideceth-9, Disodium EDTA, Fragrance, PEG-5 Ethylhexanoate, Menthol.

### Production Example 12: Benzoyl Peroxide Cream

ACTIVE INGREDIENT: Benzoyl peroxide 2.5%
INACTIVE INGREDIENTS: Water, Carbomer Homopolymer type B, Ethylhexylglycerin, Sodium Hydroxide, Chlorphenesin, Disodium EDTA, Laureth-4, Hydroxypropyl Methylcellulose

### Production Example 13: Benzoyl Peroxide Lotion

ACTIVE INGREDIENT: Benzoyl peroxide 10%
INACTIVE INGREDIENTS: water, cetyl alcohol, petrolatum, glycerin, steareth-2, zinc lactate, potassium cetyl phosphate, acrylates/C10-30 alkyl acrylate crosspolymer, xanthan gum, fragrance, benzyl alcohol, laureth-4, disodium EDTA, BHT, menthol.

### Production Example 14: Benzoyl Peroxide Gel

ACTIVE INGREDIENT: Benzoyl peroxide 10%
INACTIVE INGREDIENTS: water, carbomer homopolymer type B, hydroxypropyl methylcellulose, benzyl alcohol, disodium EDTA, laureth-4, citric acid, sodium hydroxide

### Production Example 15: Benzoyl Peroxide Gel

ACTIVE INGREDIENT: Benzoyl peroxide 2.5%
INACTIVE INGREDIENTS: Water, Carbomer Homopolymer type B, Ethylhexylglycerin, Sodium Hydroxide, Chlorphenesin, Disodium EDTA, Laureth-4, Hydroxypropyl Methylcellulose

### Production Example 16: Benzoyl Peroxide Solution

ACTIVE INGREDIENT: Benzoyl peroxide 5%
INACTIVE INGREDIENTS: Carbomer, Glycerin, Lauryl Methacrylate/Glycol Dimethacrylate Crosspolymer, Sodium C14-16 Olefin Sulfonate, Sodium Hydroxide, Water

### Production Example 17: Benzoyl Peroxide Cream

ACTIVE INGREDIENT: Benzoyl peroxide 5%
INACTIVE INGREDIENTS: Water, Cetyl Alcohol, Petrolatum, Steareth-2, Glycerin, Zinc Lactate, Potassium Cetyl Phosphate, Acrylates/C10-30 Alkyl Acrylate Crosspolymer, Polyethylene, Xanthan Gum, Benzyl Alcohol, Fragrance, Disodium EDTA, Laureth-4, BHT, Menthol

### Production Example 18: Benzoyl Peroxide Kit

ACTIVE INGREDIENT: Benzoyl peroxide 5%
INACTIVE INGREDIENTS: Water, Cetyl Alcohol, Petrolatum, Zinc Lactate, Steareth-2, Glycerin, Potassium Cetyl Phosphate, Acrylates/C10-30 Alkyl Acrylate Crosspolymer, Xanthan Gum, Benzyl Alcohol, Fragrance, Disodium EDTA, Laureth-4, BHT, Menthol

### Production Example 19: Benzoyl Peroxide Cream

ACTIVE INGREDIENT: Benzoyl peroxide 5%
INACTIVE INGREDIENTS: Water, Cetyl Alcohol, Petrolatum, Zinc Lactate, Steareth-2, Glycerin, Potassium Cetyl Phosphate, Acrylates/C10-30 Alkyl Acrylate Crosspolymer, Xanthan Gum, Benzyl Alcohol, Fragrance, Disodium EDTA, Laureth-4, BHT, Menthol

### Production Example 20: Benzoyl Peroxide Gel

ACTIVE INGREDIENT: Benzoyl peroxide 2.5%
INACTIVE INGREDIENTS: Water, Carbomer Homopolymer type B, Ethylhexylglycerin, Sodium Hydroxide, Chlorphenesin, Disodium EDTA, Laureth-4, Hydroxypropyl Methylcellulose

### Production Example 21: Benzoyl Peroxide Cream

ACTIVE INGREDIENT: Benzoyl peroxide 10%
INACTIVE INGREDIENTS: Water, Cetyl Alcohol, Petrolatum, Zinc Lactate, Steareth-2, Glycerin, Potassium Cetyl Phosphate, Acrylates/C10-30 Alkyl Acrylate Crosspolymer, Xanthan Gum, Benzyl Alcohol, Fragrance, Disodium EDTA, Laureth-4, BHT, Menthol

### Production Example 22: Benzoyl Peroxide Gel

ACTIVE INGREDIENT: Benzoyl peroxide 10%
INACTIVE INGREDIENTS: Water, Carbomer Homopolymer Type B, Hydroxypropyl Methylcellulose, Sodium Hydroxide, Laureth-4, Disodium EDTA

### Production Example 23: Benzoyl Peroxide Cream

ACTIVE INGREDIENT: Benzoyl peroxide 2.5%
INACTIVE INGREDIENTS: water, magnesium aluminum silicate, glyceryl stearate, PEG-100 stearate, sorbitol, dimethyl isosorbide, disodium PEG-12 dimethicone sulfosuccinate, tridecyl stearate, neopentyl glycol dicaprylate/dicaprate, polyethylene, cetyl esters, anthemis nobilis flower extract, sodium hyaluronate, sodium PCA, xanthan gum, tridecyl trimellitate, propylene glycol, Imidazolidinyl urea, methylparaben, propylparaben, fragrance.

### Production Example 24: Benzoyl Peroxide Lotion

ACTIVE INGREDIENT: Benzoyl peroxide 7%
INACTIVE INGREDIENTS: water (aqua), cyclotetrasiloxane, ethoxydiglycol, propylene glycol, cetearyl alcohol, glyceryl stearate, PEG-100 stearate, dimethicone, cyclopentasiloxane, panthenol, allantoin, carbomer, ceteareth-20, xanthan gum, triethanolamine, diazolidinyl urea, methylparaben, propylparaben, fragrance (parfum).

### Production Example 25: Benzoyl Peroxide Cream

ACTIVE INGREDIENT: Benzoyl peroxide 6%
INACTIVE INGREDIENTS: water (aqua), ethoxydiglycol, cyclopentasiloxane, methylpropanediol, steareth-2, cyclohexasiloxane, steareth-21, polyquaternium-37, propylene glycol, propylene glycol dicaprylate/dicaprate, PPG-1 trideceth-6, panthenol, allantoin, diazolidinyl urea, methylparaben, propylparaben, fragrance (parfum)

### Production Example 26: Benzoyl Peroxide Cream

ACTIVE INGREDIENT: Benzoyl peroxide 5%
INACTIVE INGREDIENTS: water, ethoxydiglycol, cyclopentasiloxane, methylpropanediol, steareth-2, cyclopentasiloxane, steareth-21, polyquaternium-37, propylene glycol dicaprylate/dicaprate, propylene glycol, PPG-1 trideceth-6, panthenol, allantoin, diazolidinyl urea, methylparaben, propylparaben, fragrance.

### Production Example 27: Benzoyl Peroxide Cream

ACTIVE INGREDIENT: Benzoyl peroxide 7%
INACTIVE INGREDIENTS: water, dimethyl isosorbide, disodium PEG-12 dimethicone sulfosuccinate, magnesium aluminum silicate, glyceryl stearate, PEG-100 stearate, sorbitol, tridecyl stearate, neopentyl glycol dicaprylate/dicaprate, polyethylene, cetyl esters, tridecyl trimellitate, anthemis nobilis flower extract, sodium hyaluronate, sodium PCA, propylene glycol, xanthan gum, oxidized polyethylene, trisodium sulfosuccinate, PEG-12 dimethicone, sodium sulfate, imidazolidinyl urea, methylparaben, propylparaben, fragrance, ferric ammonium ferrocyanide

### Production Example 28: Benzoyl Peroxide Gel

ACTIVE INGREDIENT: Benzoyl peroxide 8.5%
INACTIVE INGREDIENTS: acrylates/C10-30 alkyl acrylate crosspolymer, butylene glycol, coconut acid, dimethicone, ethylhexylglycerin, fragrance, glycerin, lauryl/myristyl benzoate, linoleic acid, linolenic acid, menthol, paraffin, phenoxyethanol, sodium cocoyl isethionate, sodium hydroxide, sodium isethionate, water

### Production Example 29: Benzoyl Peroxide Lotion

ACTIVE INGREDIENT: Benzoyl peroxide 7%
INACTIVE INGREDIENTS: water, ethoxydiglycol, cyclotetrasiloxane, cyclopentasiloxane, propylene glycol, cetearyl alcohol, glyceryl stearate, PEG-100 stearate, dimethicone, ceteareth-20, carbomer, panthenol, allantoin, xanthan gum, sodium hydroxide, diazolidinyl urea, methylparaben, propylparaben, fragrance.

### Production Example 30: Benzoyl Peroxide Gel

ACTIVE INGREDIENT: Benzoyl peroxide 2.5%
INACTIVE INGREDIENTS: water, ethoxydiglycol, glycerin, prunus amygdalus dulcis (sweet almond) oil, polysorbate 80, polysorbate 60, sorbitan stearate, carbomer, potassium hydroxide, disodium EDTA, fragrance

### Production Example 31: Benzoyl Peroxide Cream

ACTIVE INGREDIENT: Benzoyl peroxide 2.5%
INACTIVE INGREDIENTS: water (aqua), magnesium aluminum silicate, glyceryl stearate, PEG-100 stearate, disodium PEG-12 dimethicone sulfosuccinate, sorbitol, dimethyl isosorbide, tridecyl stearate, neopentyl glycol dicaprylate/dicaprate, polyethylene, cetyl esters, anthemis nobilis flower extract, sodium hyaluronate, sodium PCA, xanthan gum, tridecyl trimellitate, propylene glycol, Imidazolidinyl urea, methylparaben, propylparaben, fragrance (parfum)

### Production Example 32: Benzoyl Peroxide Gel

ACTIVE INGREDIENT: Benzoyl peroxide 2.5%
INACTIVE INGREDIENTS: water, magnesium aluminum silicate, glyceryl stearate, PEG-100 stearate, sorbitol, dimethyl isosorbide, disodium PEG-12 dimethicone sulfosuccinate, tridecyl stearate, neopentyl glycol dicaprylate/dicaprate, paraffin, cetyl esters, anthemis nobilis flower extract, tridecyl trimellitate trisodium sulfosuccinate, PEG-12 dimethicone, sodium sulfate, sodium hyaluronate, propylene glycol, sodium PCA, xanthan gum, phenoxyethanol, imidazolidinyl urea, methylparaben, propylparaben, fragrance

### Production Example 33: Benzoyl Peroxide Lotion

ACTIVE INGREDIENT: Benzoyl peroxide 2.5%
INACTIVE INGREDIENTS: water (aqua), ethoxydiglycol, cyclotetrasiloxane, propylene glycol, cetearyl alcohol, dimethicone, glyceryl stearate, PEG-100 stearate, cyclopentasiloxane, panthenol, allantoin, xanthan gum, ceteareth-20, carbomer, triethanolamine, diazolidinyl urea, methylparaben, propylparaben, fragrance (parfum)

### Production Example 34: Benzoyl Peroxide Cream

ACTIVE INGREDIENT: Benzoyl peroxide 5%
INACTIVE INGREDIENTS: Water, Sodium Cocoyl Isethionate, Glycerin, Lauryl/Myristyl Benzoate, Coconut Acid, Cocos Nucifera (Coconut) Fruit Extract, Cocos Nucifera (Coconut) Oil, PEG-60 Hydrogenated Castor Oil, Rosmarinus Officinalis (Rosemary) Leaf Extract, Helianthus Annuus (Sunflower) Seed Oil, Linoleic Acid, Linolenic Acid, Menthol, Sodium Isethionate, Butylene Glycol, Dimethicone, Phenethyl Alcohol, Caprylyl Glycol, Ethylhexylglycerin, Acrylates/C10-30 Alkyl Acrylate Crosspolymer, Sodium Hydroxide, Potassium Sorbate, Sodium Benzoate, Phenoxyethanol, Fragrance.

### Production Example 35: Benzoyl Peroxide Gel

ACTIVE INGREDIENT: Benzoyl peroxide 2.5%
INACTIVE INGREDIENTS: water, ethoxydiglycol, cyclotetrasiloxane, cyclopentasiloxane, propylene glycol, cetearyl alcohol, dimethicone, glyceryl stearate, PEG-100 stearate, panthenol, allantoin, xanthan gum, ceteareth-20, carbomer, sodium hydroxide, diazolidinyl urea, methylparaben, propylparaben, fragrance

### Production Example 36: Benzoyl Peroxide Gel

ACTIVE INGREDIENT: Benzoyl peroxide 2.5%
INACTIVE INGREDIENTS: water, ethoxydiglycol, cyclotetrasiloxane, cyclopentasiloxane, propylene glycol, cetearyl alcohol, dimethicone, glyceryl stearate, PEG-100 stearate, panthenol, allantoin, xanthan gum, ceteareth-20, carbomer, sodium hydroxide, diazolidinyl urea, methylparaben, propylparaben, fragrance

### Production Example 37: Benzoyl Peroxide Lotion

ACTIVE INGREDIENT: Benzoyl peroxide 2.5%
INACTIVE INGREDIENTS: water (aqua), ethoxydiglycol, cyclotetrasiloxane, propylene glycol, cetearyl alcohol, dimethicone, glyceryl stearate, PEG-100 stearate, cyclopentasiloxane, panthenol, allantoin, xanthan gum, ceteareth-20, carbomer, triethanolamine, diazolidinyl urea, methylparaben, propylparaben, fragrance (parfum)

### Production Example 38: Benzoyl Peroxide Lotion

ACTIVE INGREDIENT: Benzoyl peroxide 2.5%
INACTIVE INGREDIENTS: water (aqua), ethoxydiglycol, cyclotetrasiloxane, cyclopentasiloxane, cetearyl alcohol, propylene glycol, dimethicone, glyceryl stearate, PEG-100 stearate, panthenol, allantoin, xanthan gum, ceteareth-20, carbomer, triethanolamine, diazolidinyl urea, methylparaben, propylparaben, fragrance (parfum)

### Production Example 39: Benzoyl Peroxide Lotion

ACTIVE INGREDIENT: Benzoyl peroxide 2.5%
INACTIVE INGREDIENTS: water, magnesium aluminum silicate, glyceryl stearate, PEG-100 stearate, sorbitol, dimethyl isosorbide, disodium PEG-12 dimethicone sulfosuccinate, neopentyl glycol dicaprylate/dicaprate, tridecyl stearate, polyethylene, cetyl esters, anthemis nobilis flower extract, tridecyl trimellitate, imidazolidinyl urea, sodium PCA, propylene glycol, sodium hyaluronate, xanthan gum, methylparaben, propylparaben, fragrance

### Production Example 40: Benzoyl Peroxide Gel

ACTIVE INGREDIENT: Benzoyl peroxide 2.5%
INACTIVE INGREDIENTS: water, ethoxydiglycol, cyclotetrasiloxane, cyclopentasiloxane, cetearyl alcohol, dimethicone, glyceryl stearate, peg-100 stearate, propylene glycol, panthenol, allantoin, xanthan gum, ceteareth-20, carbomer, sodium hydroxide, diazolidinyl urea, methylparaben, propylparaben, fragrance

### Production Example 41: Benzoyl Peroxide Gel

ACTIVE INGREDIENT: Benzoyl peroxide 2.5%
INACTIVE INGREDIENTS: water, sodium cocoyl isethionate, glycerin, lauryl/myristyl benzoate, coconut acid, linoleic acid, linolenic acid, menthol, butylene glycol, dimethicone, ethylhexylglycerin, acrylates/C10-30 alkyl acrylate crosspolymer, sodium isethionate, sodium hydroxide, phenoxyethanol, fragrance

### Production Example 42: Benzoyl Peroxide Lotion

ACTIVE INGREDIENT: Benzoyl peroxide 2.5%
INACTIVE INGREDIENTS: water, sodium cocoyl isethionate, glycerin, paraffin, lauryl/myristyl benzoate, coconut acid, tocopherol, linoleic acid, linolenic acid, butylene glycol, dimethicone, ethylhexylglycerin, acrylates/c10-30 alkyl acrylate crosspolymer, glycolic acid, sodium isethionate, sodium hydroxide, phenoxyethanol, fragrance.

### Production Example 43: Benzoyl Peroxide Lotion

ACTIVE INGREDIENT: Benzoyl peroxide 2.5%
INACTIVE INGREDIENTS: water, cyclopentasiloxane, butylene glycol, ceteareth-20, dimethicone, sucrose, camellia sinensis (green tea) leaf extract, hordeum vulgare (barley) extract\extrait d'orge, acetyl glucosamine, lactobacillus ferment, poria cocos sclerotium extract, laminaria saccharina extract, polymethyl methacrylate, gentiana lutea (gentian) root extract, helianthus annuus (sunflower) seedcake, saccharomyces lysate extract, astrocaryum murumuru butter, acrylamide/sodium acryloyldimethyltaurate copolymer, myristyl alcohol, glycerin, zeolite, algae extract, linoleic acid, 10-hydroxydecanoic acid, cucumis sativus (cucumber) fruit extract, caffeine, cholesterol, tocopheryl acetate, bisabolol, longifolene, polysorbate 80, sodium hyaluronate, beta-caryophyllene, propylene glycol dicaprate, capryloyl glycine, quaternium-22, mentha piperita (peppermint), perilla aldehyde, caprylyl glycol, isohexadecane, xanthan gum, disodium edta, chloroxylenol, phenoxyethanol, titanium dioxide (ci 77891)

### Production Example 44: Benzoyl Peroxide Kit

ACTIVE INGREDIENT: Benzoyl peroxide 3.7%
INACTIVE INGREDIENTS: water, propylene glycol, glycerin, carbomer, potassium hydroxide, polysorbate 80, methyl methacrylate/glycol dimethacrylate crosspolymer, disodium EDTA, sodium lauroyl lactylate, ceramide NP, ceramide AP, phytosphingosine, cholesterol, xanthan gum, ceramide EOP

### Production Example 45: Benzoyl Peroxide Liquid

ACTIVE INGREDIENT: Benzoyl peroxide 2.5%
INACTIVE INGREDIENTS: water, glycerin, propylene glycol, cocamidopropyl hydroxysultaine, sodium C14-16 olefin sulfonate, xanthan gum, citric acid, potassium hydroxide, methyl methacrylate/glycol dimethacrylate crosspolymer, sodium lauroyl lactylate, glycolic acid, ceramide NP, ceramide AP, phytosphingosine, cholesterol, carbomer, ceramide EOP

### Production Example 46: Benzoyl Peroxide Kit

ACTIVE INGREDIENT: Benzoyl peroxide 2.5%
INACTIVE INGREDIENTS: Water, Glycerin, Propylene Glycol, Cocamidopropyl Hydroxysultaine, Sodium C14-16 Olefin Sulfonate, Xanthan Gum, Citric Acid, Potassium Hydroxide, Methyl Methacrylate/Glycol Dimethacrylate Crosspolymer, Sodium Lauroyl Lactylate, Glycolic Acid, Ceramide NP, Ceramide AP, Phytosphingosine, Cholesterol, Carbomer, Ceramide EOP

### Production Example 47: Benzoyl Peroxide Kit

ACTIVE INGREDIENT: Benzoyl peroxide 3%
INACTIVE INGREDIENTS: Water, Ethyl Macadamiate, Propanediol, Glycerin, Butylene Glycol, Polyacrylamide, Phenoxyethanol, C13-14 Isoparaffin, Bisabolol, Laureth-7, Allantoin, Melaleuca Alternifolia (Tea Tree) Leaf Oil, Methylisothiazolinone, Sodium Hyaluronate, Methyl Methacrylate/Glycol Dimethacrylate Crosspolymer, Sodium Hydroxide, Sea Whip Extract, Zingiber Officinale (Ginger) Root Extract

### Production Example 48: Salicyclic Acid Gel

ACTIVE INGREDIENT: Salicylic Acid 2%
INACTIVE INGREDIENTS: Water, glycerin, sodium laureth sulfate, cocamidopropyl betaine, coco-glucoside, decyl glucoside, PEG-200 hydrogenated glyceryl palmate, laureth 4, PEG-7 glyceryl cocoate, polyquaternium 43, fragrance, sodium hydroxide, phenoxyethanol, sodium benzoate, lavandula stoechas extract, helichrysum italicum extract, cistus monspeliensis extract, blue 1

### Production Example 49: Salicyclic Acid Lotion

ACTIVE INGREDIENT: Salicylic Acid 2%
INACTIVE INGREDIENTS: water, butylene glycol, octyldodecanol, steareth-2, cetyl alcohol, steareth-21, dimethicone, polyacrylamide, glycerin, C13-14 isoparaffin, sodium hydroxide, xanthan gum, magnesium aluminum silicate, laureth-7, fragrance, lavandula stoechas extract, helichrysum italicum extract, cistus monspeliensis extract, titanium dioxide

### Production Example 50: Salicyclic Acid Lotion

ACTIVE INGREDIENT: Salicylic Acid 2%
INACTIVE INGREDIENTS: Water, Sodium Laureth Sulfate, Polyethylene, Propylene Glycol, Polyacrylate-1 Crosspolymer, Cocamidopropyl Betaine, Fragrance, Sorbitol, Sodium Chloride, Citric Acid, Polyquaternium 43, Phenoxyethanol, Methylparaben, Butylparaben, Ethylparaben, Isobutylparaben, Propylparaben, FD & C Blue No. 1 Lake.

### Production Example 51: Salicyclic Acid Lotion

ACTIVE INGREDIENT: Salicylic Acid 2%
INACTIVE INGREDIENTS: Water, sodium laureth sulfate, polyethylene, propylene glycol, polyacrylate-1 crosspolymer, cocamidopropyl betaine, fragrance, sorbitol, sodium chloride, citric acid, polyquaternium 43, phenoxyethanol, sodium benzoate, FD&C blue no. 1 lake

### Production Example 52: Salicyclic Acid Swab

ACTIVE INGREDIENT: Salicylic Acid 2%
INACTIVE INGREDIENTS: Water, Alcohol Denat, Isoceteth-20, Aloe Barbadensis Leaf Juice, Sodium Hydroxide, Triethanolamine, Allantoin, Fragrance, Menthol, Disodium EDTA, Sodium Benzoate, Potassium Sorbate, Citric Acid

### Production Example 53: Salicyclic Acid Gel

ACTIVE INGREDIENT: Salicylic Acid 2%
INACTIVE INGREDIENTS: water, sodium laureth sulfate, hydrogenated starch hydrolysate, cocamidopropyl betaine, ammonium lauryl sulfate, sodium chloride, sodium cocoamphoacetate, fragrance, sodium hydroxide, citric acid, tetrasodium EDTA, buteth-3, sodium benzotriazolyl butylphenol sulfonate, polyquaternium-43, tributyl citrate, magnesium nitrate, phenoxyethanol, sodium benzoate, methylchloroisothiazolinone, methylisothiazolinone, FD&C blue no. 1, D&C red no. 33

### Production Example 54: Salicyclic Acid Lotion

ACTIVE INGREDIENT: Salicylic Acid 2%
INACTIVE INGREDIENTS: water, sodium laureth sulfate, polyethylene, propylene glycol, polyacrylate-1 crosspolymer, cocamidopropyl betaine, fragrance, sorbitol, sodium chloride, citric acid, polyquaternium 43, phenoxyethanol, sodium benzoate, FD&C blue no. 1 lake

### Production Example 55: Salicyclic Acid Lotion

ACTIVE INGREDIENT: Salicylic Acid 2%
INACTIVE INGREDIENTS: water, sodium laureth sulfate, polyethylene, propylene glycol, polyacrylate-1 crosspolymer, cocamidopropyl betaine, hydrogenated starch hydrolysate, fragrance, sodium chloride, citric acid, polyquaternium-43, phenoxyethanol, sodium benzoate, FD&C blue no. 1 lake

### Production Example 56: Salicyclic Acid Lotion

ACTIVE INGREDIENT: Salicylic Acid 2%
INACTIVE INGREDIENTS: water, glycerin, myristic acid, stearic acid, sodium lauroyl sarcosinate, palmitic acid, potassium hydroxide, lauric acid, polyquaternium-10, fragrance, phenoxyethanol, tetrasodium EDTA, pentasodium pentetate, tetrasodium etidronate, menthol, methylparaben, butylparaben, ethylparaben, isobutylparaben, propylparaben, Aloe barbadensis leaf juice, maltodextrin, potassium sorbate, sodium benzoate

### Production Example 57: Salicyclic Acid Lotion

ACTIVE INGREDIENT: Salicylic Acid 2%
INACTIVE INGREDIENTS: water, glycerin, myristic acid, stearic acid, sodium lauroyl sarcosinate, palmitic acid, potassium hydroxide, lauric acid, polyquaternium-10, phenoxyethanol, tetrasodium EDTA, pentasodium pentetate, tetrasodium etidronate, methylparaben, butylparaben, ethylparaben, isobutylparaben, propylparaben, Aloe barbadensis leaf extract, maltodextrin, potassium sorbate, sodium benzoate

### Production Example 58: Salicyclic Acid Solution

ACTIVE INGREDIENT: Salicylic Acid 2%
INACTIVE INGREDIENTS: water, alcohol denat, isoceteth-20, Aloe barbadensis leaf juice, sodium hydroxide, triethanolamine, allantoin, fragrance, menthol, disodium EDTA, potassium sorbate, sodium benzoate, citric acid

### Production Example 59: Salicyclic Acid Solution

ACTIVE INGREDIENT: Salicylic Acid 1%
INACTIVE INGREDIENTS: water, alcohol denat., isoceteth-20, glycerin, sorbitol, sodium hydroxide, fragrance, niacinamide, Aloe barbadensis leaf juice, butylene glycol, Cucumis sativus (cucumber) fruit extract, menthol, citric acid, potassium sorbate, sodium benzoate

### Production Example 60: Salicyclic Acid Lotion

ACTIVE INGREDIENT: Salicylic Acid 2%
INACTIVE INGREDIENTS: water, glycerin, myristic acid, stearic acid, sodium lauroyl sarcosinate, palmitic acid, potassium hydroxide, lauric acid, polyquaternium 10, fragrance, phenoxyethanol, tetrasodium EDTA, pentasodium pentetate, tetrasodium etidronate, menthol, methylparaben, butylparaben, ethylparaben, isobutylparaben, propylparaben, aloe barbadensis leaf juice, maltodextrin, potassium sorbate, sodium benzoate

### Production Example 61: Salicyclic Acid Lotion

ACTIVE INGREDIENT: Salicylic Acid 2%
INACTIVE INGREDIENTS: water, glycerin, myristic acid, stearic acid, sodium lauroyl sarcosinate, palmitic acid, potassium hydroxide, lauric acid, caprylyl glycol, polyquaternium-10, fragrance, ethylhexylglycerin, tetrasodium etidronate, tetrasodium EDTA, menthol, Aloe barbadensis leaf juice, maltodextrin, disodium phosphate, potassium sorbate, sodium benzoate

### Production Example 62: Salicyclic Acid Lotion

ACTIVE INGREDIENT: Salicylic Acid 2%
INACTIVE INGREDIENTS: water, glycerin, myristic acid, stearic acid, sodium lauroyl sarcosinate, palmitic acid, potassium hydroxide, lautic acid, caprylyl glycol, polyquaternium-10, ethylhexylglycerin, tetrasodium etidronate, tetrasodium EDTA, Aloe barbadensis leaf juice, maltodextrin, disodium phosphate, potassium sorbate, sodium benzoate

### Production Example 63: Salicyclic Acid Lotion

ACTIVE INGREDIENT: Salicylic Acid 2%
INACTIVE INGREDIENTS: water, glycerin, myristic acid, stearic acid, sodium lauroyl sarcosinate, palmitic acid, potassium hydroxide, lauric acid, polyquaternium 10, phenoxyethanol, tetrasodium EDTA, pentasodium pentetate, tetrasodium etidronate, methylparaben, butylparaben, ethylparaben, isobutylparaben, propylparaben, aloe barbadensis leaf extract, maltodextrin, potassium sorbate, sodium benzoate

### Production Example 64: Salicyclic Acid Gel

ACTIVE INGREDIENT: Salicylic Acid 2%
INACTIVE INGREDIENTS: water, sodium laureth sulfate, sorbitol, cocamidopropyl betaine, ammonium lauryl sulfate, sodium chloride, sodium cocoamphoacetate, fragrance, sodium hydroxide, citric acid, tetrasodium EDTA, buteth-3, sodium benzotriazolyl butylphenol sulfonate, polyquaterniutn-43, tributyl citrate, magnesium nitrate, phenoxyethanol, sodium benzoate, methylchloroisothiazolinone, methylisothiazolinone, FD&C blue no. 1, D&C red no. 33

### Production Example 65: Salicyclic Acid Solution

ACTIVE INGREDIENT: Salicylic Acid 2%
INACTIVE INGREDIENTS: Water, PPG-15 Stearyl Ether, Glycerin, Stearyl Alcohol, Cetyl Betaine, Distearyldimonium Chloride, Sodium Lauryl Sulfate, Oxidized Polyethylene, Cetyl Alcohol, Alcohol, Steareth-21, Sodium Chloride, Sodium Hydroxide, Behenyl Alcohol, Fragrance, PPG-30, Steareth-2, Dipropylene Glycol, Disodium EDTA, BHT.

### Production Example 66: Salicyclic Acid Solution

ACTIVE INGREDIENT: Salicylic Acid 1%
INACTIVE INGREDIENTS: Water, Alcohol Denat., Isoceteth-20, Glycerin, Hydrogenated Starch Hydrolysate, Sodium Hydroxide, Fragrance, Niacinamide, Aloe Barbadensis Leaf Juice, Butylene Glycol, Cucumis Sativus (Cucumber) Fruit Extract, Menthol, Citric Acid, Potassium Sorbate, Sodium Benzoate

### Production Example 67: Salicyclic Acid Lotion

ACTIVE INGREDIENT: Salicylic Acid 1%
INACTIVE INGREDIENTS: Water, Sodium Cocoyl Isethionate, Glycerin, Cetyl Alcohol, Sodium Lauroyl Sarcosinate, Stearyl Alcohol, Cocamidopropyl Betaine, Steareth-21, Steareth-2, Coco-Glucoside, Fragrance, Acrylates/C 10-30 Alkyl Acrylate Crosspolymer, Sodium Hydroxide, Niacinamide, Aloe Barbadensis Leaf Juice, Disodium EDTA, Butylene Glycol, Cucumis Sativus (Cucumber) Fruit Extract, Potassium Sorbate, Sodium Benzoate

### Production Example 68: Salicyclic Acid Kit

ACTIVE INGREDIENT: Salicylic Acid 2%
INACTIVE INGREDIENTS: Water, Glycerin, Sodium Laureth Sulfate, Cocamidopropyl Betaine, Coco-Glucoside, Glycol Distearate, Sodium Chloride, Laureth-4, Tocopheryl Acetate, Polyquatemium-43, Fragrance, Propylene Glycol, Formic Acid, Phenoxyethanol, Methylparaben, Butylparaben, Ethylparaben, Isobutylparaben, Propylparaben, Lavandula Stoechas Extract, Helichrysum Italicum Extract, Cistus Monspeliensis Extract, FD&C Blue No. 1, FD&C Yellow No. 6, FD&C Yellow No. 5

### Production Example 69: Salicyclic Acid Kit

ACTIVE INGREDIENT: Salicylic Acid 2%
INACTIVE INGREDIENTS: water, glycerin, sodium laureth sulfate, cocamidopropyl betaine, coco-glucoside, glycol distearate, sodium chloride, laureth-4, tocopheryl acetate, polyquatemium-43, fragrance, propylene glycol, formic acid, phenoxyethanol, methytparaben, butylparaben, ethylparaben, isobutylparaben, propylparaben, lavandula stoechas extract, helichrysum italicum extract, cistus monspeliensis extract, FD&C blue no. 1, FD&C yellow no. 6, FD&C yellow no. 5

### Production Example 70: Salicyclic Acid Lotion

ACTIVE INGREDIENT: Salicylic Acid 2%
INACTIVE INGREDIENTS: Water, Glycerin, Sodium Laureth Sulfate, Cocamidopropyl Betaine, Coco-Glucoside, Glycol Distearate, Sodium Chloride, Laureth-4, Tocopheryl Acetate, Polyquatemium-43, Fragrance, Propylene Glycol, Formic Acid, Phenoxyethanol, Methylparaben, Butylparaben, Ethylparaben, Isobutylparaben, Propylparaben, Lavandula Stoechas Extract, Helichrysum Italicum Extract, Cistus Monspeliensis Extract, FD&C Blue No.1, FD&C Yellow No.6, FD&C Yellow No.5

### Production Example 71: Salicyclic Acid Lotion

ACTIVE INGREDIENT: Salicylic Acid 2%
INACTIVE INGREDIENTS: Water, Glycerin, Sodium Laureth Sulfate, Cocamidopropyl Betaine, Coco-Glucoside, Sodium Chloride, Laureth-4, Fragrance, Tocopheryl Acetate, Propylene Glycol, Polyquaternium-43, Disodium EDTA, Trideceth-9, Rubus Idaeus (Raspberry Juice, Vaccinium Macrocarpon (Cranberry) Fruit Extract, Phenoxyethanol, Bisabolol, Citric Acid, Methylparaben, Butylparaben, Ethylparaben, Isobutylparaben, Propylparaben, D&C Red No. 33, FD&C Yellow No. 6

### Production Example 72: Salicyclic Acid Lotion

ACTIVE INGREDIENT: Salicylic Acid 2%
INACTIVE INGREDIENTS: Water, Butylene Glycol, Octyldodecanol, Steareth-2, Cetyl Alcohol, Steareth-21, Dimethicone, Polyacrylamide, Glycerin, C13-14 Isoparaffin, Sodium Hydroxide, Xanthan Gum, Magnesium Aluminum Silicate, Laureth-7, Fragrance, Lavandula Stoechas Extract, Helichrysum Italicum Extract, Cistus Monspeliensis Extract, Titanium Dioxide

### Production Example 73: Salicyclic Acid Cloth

ACTIVE INGREDIENT: Salicylic Acid 2%
INACTIVE INGREDIENTS: Water, Alcohol Denat., Glycerin, Isoceteth-20, Hydrogenated Starch Hydrolysate, Sodium Hydroxide, Fragrance, Lavandula Stoechas Extract, Helichrysum Italicum Extract, Cistus Monspeliensis Extract, Disodium EDTA

### Production Example 74: Salicyclic Acid Lotion

ACTIVE INGREDIENT: Salicylic Acid 2%
INACTIVE INGREDIENTS: Water, PPG-15 Stearyl Ether, Hydrated Silica, Glycerin, Stearyl Alcohol, Cetyl Betaine, Distearyldimonium Chloride, Sodium Lauryl Sulfate, Cetyl Alcohol, Alcohol Denat, Steareth-21, Sodium Chloride, Behenyl Alcohol, Synthetic Wax, Steareth-2, Xanthan Gum, Fragrance, MEK, Disodium EDTA, Isopropyl Alcohol, BHT, Lavandula Stoechas Extract, Methylchloroisothiazolinone, Helichrysum Italicum Extract, Methylisothiazolinone, Cistus Monspeliensis Extract, Mica, Ferric Ferrocyanide, Titanium Dioxide, FD&C Blue No. 1

### Production Example 75: Salicyclic Acid Lotion

ACTIVE INGREDIENT: Salicylic Acid 2%
INACTIVE INGREDIENTS: Water, Sodium Cocoyl isethionate, Cetearyl Alcohol, Glycerin, Sodium Laureth Sulfate, Sodium Cocoatnphoacetate, Cocamidopropyl Betaine, Sodium Hydroxide, Acrylates/C10-30 Alkyl Acrylate Crosspolymer, Fragrance, Disodium EDTA, Lavendula Stoechas Extract, Helichrysum Italicum Extract, Cistus Monspeliensis Extract

### Production Example 76: Salicyclic Acid Gel

ACTIVE INGREDIENT: Salicylic Acid 2%
INACTIVE INGREDIENTS: Water, Alcohol Denat., Polysorbate 20, Sorbitol, Hydroxyethylcellulose, Glycerin, Sodium Hydroxide, Fragrance, Lavandula Stoechas Extract, Helichrysum Italicum Extract, Cistus Monspeliensis Extract, Menthol, Disodium EDTA

### Production Example 77: Salicyclic Acid Cloth

ACTIVE INGREDIENT: Salicylic Acid 1.9%
INACTIVE INGREDIENTS: Water, Alcohol Denat, Isoceteth-20, Aloe Barbadensis Leaf Juice, Sodium Hydroxide, Triethanolamine, Allantoin, Fragrance, Menthol, Disodium EDTA, Potassium Sorbate, Sodium Benzoate, Citric Acid

### Production Example 78: Salicyclic Acid Lotion

ACTIVE INGREDIENT: Salicylic Acid 2%
INACTIVE INGREDIENTS: Water, PPG-15 Stearyl Ether, Hydrated Silica, Glycerin, Stearyl Alcohol, Cetyl Betaine, Distearyldimonium Chloride, Sodium Lauryl Sulfate, Cetyl Alcohol, Alcohol Denat, Steareth-21, Sodium Chloride, Behenyl Alcohol, Synthetic Wax, Steareth-2, Xanthan Gum, Fragrance, MEK, Disodium EDTA, Isopropyl Alcohol, BHT, Lavandula Stoechas Extract, Methylchloroisothiazolinone, Helichrysum Italicum Extract, Methylisothiazolinone, Cistus Monspeliensis Extract, Mica, Ferric Ferrocyanide, Titanium Dioxide, FD&C Blue No. 1

### Production Example 79: Salicyclic Acid Lotion

ACTIVE INGREDIENT: Salicylic Acid 2%
INACTIVE INGREDIENTS: Water, Kaolin, Bentonite, Glycerin, Sodium Laureth Sulfate, Titanium Dioxide, Niacinamide, Sodium Lactate, Xanthan Gum, Sodium Hydroxide, Cocamidopropyl Betaine, Coco-Glucoside, Hexyldecanol, Fragrance, Dipotassium Glycyrrhizate, Disodium EDTA, Bisabolol, Cetylhydroxyproline Palmitamide, Stearic Acid, Brassica Campestris (Rapeseed) Sterols, FD&C Blue No. 1, FD&C Yellow No. 6

### Production Example 80: Salicyclic Acid Lotion

ACTIVE INGREDIENT: Salicylic Acid 2%
INACTIVE INGREDIENTS: Water, Sodium Cocoyl Isethionate, Glycerin, Cetyl Alcohol, Laureth-3, Sodium Laureth Sulfate, Hydrated Silica, Niacinamide, Sodium Lactate, Sodium Cocoamphoacetate, Sorbitol, Hexyldecanol, Sodium Chloride, Fragrance, Sodium Hydroxide, Acrylates/C10-30 Alkyl Acrylate Crosspolymer, Disodium EDTA, Bisabolol, Dipotassium Glycyrrhizinate, Benzyl Salicylate, Brassica Campestris (Rapeseeed) Sterols, Cetylhydroxyproline Palmitamide, Stearic Acid, Polyquaternium-43, Phenoxyethanol, Sodium Benzoate, Sodium Sulfate, FD&C Blue 1, FD&C Yellow 6

### Production Example 81: Salicyclic Acid Lotion

ACTIVE INGREDIENT: Salicylic Acid 2%
INACTIVE INGREDIENTS: Water, Kaolin, Bentonite, Glycerin, Sodium Laureth Sulfate, Titanium Dioxide, Niacinamide, Sodium Lactate, Xanthan Gum, Sodium Hydroxide, Cocamidopropyl Betaine, Coco-Glucoside, Hexyldecanol, Fragrance, Dipotassium Glycyrrhizate, Disodium EDTA, Bisabolol, Cetylhydroxyproline Palmitamide, Stearic Acid, Brassica Campestris (Rapeseed) Sterols, FD&C Blue No. 1, FD&C Yellow No. 6

### Production Example 82: Salicyclic Acid Solution

ACTIVE INGREDIENT: Salicylic Acid 2%
INACTIVE INGREDIENTS: Water, Sodium Laureth Sulfate, Sorbitol, Cocamidopropyl Betaine, Ammonium Lauryl Sulfate, Sodium Chloride, Sodium Hydroxide, Sodium Cocoamphoacetate, Fragrance, Tetrasodium EDTA, Lavandula Stoechas Extract, Helichrysum Italicum Extract, Cistus Monspelienses Extract, Polyquaternium-43, Magnesium Nitrate, PEG-40 Hydrogenated Castor Oil, Phenoxyethanol, Methylparaben, Methylchloroisothiazolinone, Magnesium Chloride, Methylisothiazolinone, Butylparaben, Ethylparaben, Isobutylparaben, Propylparaben, Blue 1.

### Production Example 83: Salicyclic Acid Solution

ACTIVE INGREDIENT: Salicylic Acid 2%
INACTIVE INGREDIENTS: Water, Sodium Laureth Sulfate, Sorbitol, Cocamidopropyl Betaine, Ammonium Lauryl Sulfate, Sodium Chloride, Sodium Hydroxide, Sodium Cocoamphoacetate, Fragrance, Tetrasodium EDTA, Lavandula Stoechas Extract, Helichrysum Italicum Extract, Cistus Monspelienses Extract, Polyquaternium-43, Magnesium Nitrate, PEG-40 Hydrogenated Castor Oil, Phenoxyethanol, Methylparaben, Methylchloroisothiazolinone, Magnesium Chloride, Methylisothiazolinone, Butylparaben, Ethylparaben, Isobutylparaben, Propylparaben, Blue 1.

### Production Example 84: Salicyclic Acid Solution

ACTIVE INGREDIENT: Salicylic Acid 2%
INACTIVE INGREDIENTS: Water, Sodium Cocoyl Isethionate, Cetearyl Alcohol, Laureth-3, Glycerin, Coconut Acid, Sodium Isethionate, Sodium Hydroxide, Fragrance, Lavandula Stoechas Extract, Helichrysum Italicum Extract, Cistus Monspeliensis Extract, Disodium EDTA, PEG-40 Hydrogenated Castor Oil, Phenoxyethanol, Methylparaben, Butylparaben, Ethylparaben, Isobutylparaben, Propylparaben

### Production Example 85: Salicyclic Acid Solution

ACTIVE INGREDIENT: Salicylic Acid 2%
INACTIVE INGREDIENTS: water, butylene glycol, octyldodecanol, steareth-2, cetyl alcohol, steareth-21, dimethicone, polyacrylamide, glycerin, C13-14 isoparaffin, sodium hydroxide, xanthan gum, magnesium aluminum silicate, laureth-7, fragrance, Lavandula stoechas extract Helichrysum italicum extract, Cistus monspeliensis extract, titanium dioxide

### Production Example 86: Salicyclic Acid Solution

ACTIVE INGREDIENT: Salicylic Acid 2%
INACTIVE INGREDIENTS: water, polyethylene, PPG-15 stearyl ether, glycerin, stearyl alcohol, cetyl betaine, distearyldimonium chloride, sodium lauryl sulfate, cetyl alcohol, alcohol, steareth-21, sodium chloride, behenyl alcohol, synthetic wax, steareth-2, fragrance, Lavandula stoechas extract, Helichrysum italicum extract, Cistus monspeliensis extract, xanthan gum, dimethyl palmitamine, lauryl alcohol, mica, isopropyl alcohol, disodium EDTA, BHT, magnesium nitrate, methylchloroisothiazolinone, magnesium chloride, methylisothiazolinone, sodium sulfate, ferric ferrocyanide, titanium dioxide, FD&C blue no. 1

### Production Example 87: Salicyclic Acid Solution

ACTIVE INGREDIENT: Salicylic Acid 1.9%
Inactive ingredients: water, alcohol denat, isoceteth-20, Aloe barbadensis leaf juice, sodium hydroxide, triethanolamine, allantoin, fragrance, menthol, disodium EDTA, potassium sorbate, sodium benzoate, citric acid

### Production Example 88: Salicyclic Acid Cream

ACTIVE INGREDIENT: Salicylic Acid 2%
Inactive ingredients: water, PPG-15 stearyl ether, hydrated silica, glycerin, stearyl alcohol, cetyl betaine, distearyldimonium chloride, sodium lauryl sulfate, cetyl alcohol, alcohol denat, steareth-21, sodium chloride, behenyl alcohol, synthetic wax, steareth-2, fragrance, Lavandula stoechas extract, Helichrysum italicum extract, Cistus monspeliensis extract, xanthan gum, dimethyl palmitamine, lauryl alcohol, mica, isopropyl alcohol, disodium EDTA, BHT, magnesium nitrate, methylchloroisothiazolinone, magnesium chloride, methylisothiazolinone, sodium sulfate, ferric ferrocyanide, titanium dioxide, FD&C blue no. 1

### Production Example 89: Salicyclic Acid Solution

ACTIVE INGREDIENT: Salicylic Acid 2%
INACTIVE INGREDIENTS: Water, Polyethylene, PPG-15 Stearyl Ether, Glycerin, Stearyl Alcohol, Cetyl Betaine, Distearyldimonium Chloride, Sodium Lauryl Sulfate, Cetyl Alcohol, Alcohol, Steareth-21, Sodium Chloride, Behenyl Alcohol, Synthetic Wax, Steareth-2, Fragrance, Xanthan Gum, Lavandula Stoechas Extract, Helichrysum Italicum Extract, Cistus Monspeliensis Extract, Lauryl Alcohol, Mica, Disodium EDTA, PEG-40 Hydrogenated Castor Oil, BHT, Magnesium Nitrate, Phenoxyethanol, Methylparaben, Methylchloroisothiazolinone, Magnesium Chloride, Butylparaben, Ethylparaben, Isobutylparaben, Propylparaben, Methylisothiazolinone, Ferric Ferrocyanide, Titanium Dioxide, Blue1.

### Production Example 90: Salicyclic Acid Lotion

ACTIVE INGREDIENT: Salicylic Acid 2%
INACTIVE INGREDIENTS: water, sodium cocoyl isethionate, glycerin, cetyl alcohol, laureth-3, polyethylene, sodium laureth sulfate, sodium cocoamphoacetate, niacinamide, sodium lactate, sorbitol, hexyldecanol, fragrance, acrylates C10-30 alkyl acrylate crosspolymer, sodium hydroxide, dipotassium glycrrhizate, disodium EDTA, cetylhydroxyproline palmitamide, stearic acid, polyquaternium-43, brassica campestris (rapeseed) sterols, phenoxyethanol, methylparaben, butylparaben, ethylparaben, isobutylparaben, propylparaben, FD&C blue no. 1, FD&C yellow no. 6

### Production Example 91: Salicyclic Acid Lotion

ACTIVE INGREDIENT: Salicylic Acid 2%
INACTIVE INGREDIENTS: water, sodium cocoyl isethionate, glycerin, cetyl alcohol, laureth-3, polyethylene, sodium laureth sulfate, sodium cocoamphoacetate, niacinamide, sodium lactate, sorbitol, hexyldecanol, fragrance, acrylates/C10-30 alkyl acrylate crosspolymer, sodium hydroxide, dipotassium glycyrrhizate, disodium EDTA, bisabolol, cetylhydroxyproline palmitamide, stearic acid, polyquaternium-43, Brassica campestris (rapeseed) sterols, phenoxyethanol, sodium benzoate, FD&C blue no. 1, FD&C yellow no. 6

### Production Example 92: Salicyclic Acid Solution

ACTIVE INGREDIENT: Salicylic Acid 2%
INACTIVE INGREDIENTS: water, alcohol denat., glycerin, isoceteth-20, sodium hydroxide, fragrance, Lavandula stoechas extract, Helichrysum italicum extract, Cistus monspeliensis extract, disodium EDTA

### Production Example 93: Salicyclic Acid Lotion

ACTIVE INGREDIENT: Salicylic Acid 2%
INACTIVE INGREDIENTS: Water, Alcohol Denat., Octyldodecanol, Dimethicone, Niacinamide, Sodium Lactate, Isohexadecane, Polyacrylamide, Sodium Hydroxide, Acrylates/C10-30 Alkyl Acrylate Crosspolymer, Xanthan Gum, Hexyldecanol, C13-14 Isoparaffin, Magnesium Aluminum Silicate, Fragrance, Laureth-7, Disodium EDTA, Dipotassium Glycyrrhizate, Bisabolol, Cetylhydroxyproline Palmitamide, Stearic Acid, Brassica Campestris (Rapeseed) Sterols, Titanium Dioxide

### Production Example 94: Salicyclic Acid Solution

ACTIVE INGREDIENT: Salicylic Acid 2%
INACTIVE INGREDIENTS: water, butylene glycol, octyldodecanol, steareth-2, cetyl alcohol, steareth-21, dimethicone, polyacrylamide, glycerin, C13-14 isoparaffin, sodium hydroxide, xanthan gum, magnesium aluminum silicate, laureth-7, fragrance, Lavandula stoechas extract, Helichrysum italicum extract, Cistus monspeliensis extract, titanium dioxide

### Production Example 95: Salicyclic Acid Lotion

ACTIVE INGREDIENT: Salicylic Acid 2%
INACTIVE INGREDIENTS: water, bentonite, kaolin, glycerin, titanium dioxide, sodium laureth sulfate, niacinamide, sodium lactate, xanthan gum, coco-glucoside, cocamidopropyl betaine, hexyldecanol, sodium hydroxide, fragrance, dipotassium glycyrrhizate, disodium EDTA, bisabolol, cetylhydroxyproline palmitamide, stearic acid, Brassica campestris sterols, FD&C blue no. 1, FD&C yellow no. 6

### Production Example 96: Salicyclic Acid Cream

ACTIVE INGREDIENT: Salicylic Acid 2%
INACTIVE INGREDIENTS: water, PPG-15 stearyl ether, hydrated silica, glycerin, stearyl alcohol, cetyl betaine, distearyldimonium chloride, sodium lauryl sulfate, cetyl alcohol, alcohol, steareth-21, sodium chloride, behenyl alcohol, synthetic wax, steareth-2, fragrance, Lavandula stoechas extract, Helichrysum italicum extract, Cistus monspeliensis extract, xanthan gum, dimethyl palmitamine, lauryl alcohol, mica, isopropyl alcohol, disodium EDTA, BHT, magnesium nitrate, methylchloroisothiazolinone, magnesium chloride, methylisothiazolinone, sodium sulfate, ferric ferrocyanide, titanium dioxide, FD&C blue no. 1

### Production Example 97: Salicyclic Acid Solution

ACTIVE INGREDIENT: Salicylic Acid 2%
INACTIVE INGREDIENTS: Water, Sodium Cocoyl Isethionate, Cetearyl Alcohol, Laureth-3, Glycerin, Coconut Acid, Sodium isethionate, Sodium Hydroxide, Fragrance, Lavandula Stoechas Extract, Helchrysum Italicum Extract, Cistus Monspeliensis Extract, Disodium EDTA, PEG-40 Hydrogenated Castor Oil, Phenoxyethanol, Methylparaben, Butylparaben, Ethylparaben, Isobutylparaben, Propylparaben.

### Production Example 98: Salicyclic Acid Solution

ACTIVE INGREDIENT: Salicylic Acid 2%
INACTIVE INGREDIENTS: water, PPG-14 butyl ether, ceteareth-20, cetearyl alcohol, cetearyl isononanoate, phenoxyethanol, glycerin, glyceryl stearate, polysorbate 20, fragrance, ceteareth-12, cetyl palmitate, benzoic acid, disodium EDTA, sodium hydroxide, dehydroacetic acid, BHT

### Production Example 99: Salicyclic Acid Lotion

ACTIVE INGREDIENT: Salicylic Acid 2%
INACTIVE INGREDIENTS: Water, Butylene Glycol, Octyldodecanol, Steareth-2, Cetyl Alcohol, Steareth-21, Dimethicone, Polyacrylamide Glycerin, C13-14 Isoparaffin, Phenoxyethanol, Sodium Hydroxide, Xanthan Gum, Magnesium Aluminum Silicate, Laureth-7, Fragrance, Lavandula Stoechas Extract, Helichrysum Italicum Extract, Cistus Monspeliensis Extract, Methyl paraben, Butytparaben, Ethylparaben, Isobutylparaben, Propylparaben, Titanium Dioxide

### Production Example 100: Salicyclic Acid Solution

ACTIVE INGREDIENT: Salicylic Acid 2%
INACTIVE INGREDIENTS: Water, PPG-15 Stearyl Ether, Glycerin, Stearyl Alcohol, Cetyl Betaine, Polyethylene, Distearyldimonium Chloride, Sodium Lauryl Sulfate, Sodium Hydroxide, Cetyl Alcohol, Alcohol, Steareth-21, Sodium Chloride, Behenyl Alcohol, PPG-30, Steareth-2, Fragrance, Disodium EDTA, Isopropanol, BHT, Zinc Stearate, Red 30, Blue 1 Lake, Red 33, Blue 1.

### Production Example 101: Salicyclic Acid Solution

ACTIVE INGREDIENT: Salicylic Acid 2%
INACTIVE INGREDIENTS: water, glycerin, sodium laureth sulfate, coco-glucoside, cocamidopropyl betaine, glycol distearate, PEG-200 hydrogenated glyceryl palmate, laureth-4, sodium chloride, PEG-7 glyceryl cocoate, polyquaternium-43, fragrance, lavandula stoechas extract, helichrysum italicum extract, cistus monspeliensis extract, sodium hydroxide, formic acid, phenoxyethanol, sodium benzoate, D&C red no. 33, FD&C blue no. 1

### Production Example 102: Salicyclic Acid Solution

ACTIVE INGREDIENT: Salicylic Acid 2%
INACTIVE INGREDIENTS: Water, Glycerin, Sodium Laureth Sulfate, Coco-Glucoside, Cocamidopropyl Betaine, Glycol Distearate, Sodium Chloride, PEG-200 Hydrogenated Glyceryl Palmate, Laureth-4, PEG-7 Glyceryl Cocoate, Polyquaternium-43, Fragrance, Lavandula Stoechas Extract, Helichrysum Italicum Extract, Cistus Monspeliensis Extract, Sodium Hydroxide, Formic Acid, Phenoxyethanol, Methylparaben, Butylparaben, Ethylparaben, Isobutylparaben, Propylparaben, Red 33, Blue 1.

### Production Example 103: Salicyclic Acid Cream

ACTIVE INGREDIENT: Salicylic Acid 2%
INACTIVE INGREDIENTS: water, glycerin, myristic acid, stearic acid, sodium lauroyl sarcosinate, palmitic acid, potassium hydroxide, lauric acid, polyquaternium-10, fragrance, phenoxyethanol, tetrasodium EDTA, pentasodium pentetate, tetrasodium etidronate, menthol, methylparaben, butylparaben, ethylparaben, isobutylparaben, propylparaben, Aloe barbadensis leaf juice, maltodextrin, potassium sorbate, sodium benzoate.

### Production Example 104: Salicyclic Acid Lotion

ACTIVE INGREDIENT: Salicylic Acid 2%
INACTIVE INGREDIENTS: water, sodium cocoyl isethionate, cetearyl alcohol, glycerin, sodium laureth sulfate, sodium cocoamphoacetate, cocamidopropyl betaine, sodium hydroxide, acrylates/C10-30 alkyl acrylate crosspolymer, fragrance, disodium EDTA, Lavendula stoechas extract, Helichrysum italicum extract, Cistus monspeliensis extract.

### Production Example 105: Salicyclic Acid Gel

ACTIVE INGREDIENT: Salicylic Acid 2%
INACTIVE INGREDIENTS: water, glycerin, sodium laureth sulfate, coca-glucoside, cocamidopropyl betaine, PEG-200 hydrogenated glyceryl palmate, sodium chloride, laureth-4, PEG-7 glyceryl cocoate, polyquaternium-43, fragrance, lavandula stoechas extract, helichrysum italicum extract, cistus monspeliensis extract, sodium hydroxide, phenoxyethanol, sodium benzoate, FD&C blue no. 1

### Production Example 106: Salicyclic Acid Gel

ACTIVE INGREDIENT: Salicylic Acid 2%
INACTIVE INGREDIENTS: water, glycerin, sodium laureth sulfate, coca-glucoside, cocamidopropyl betaine, PEG-200 hydrogenated glyceryl palmate, sodium chloride, laureth-4, PEG-7 glyceryl cocoate, polyquaterium-43, fragrance, Lavandula stoechas extract, Helichrysum italicum extract, Cistus monspeliensis extract, sodium hydroxide, phenoxyethanol, sodium benzoate, FD&C blue no. 1.

### Production Example 107: Salicyclic Acid Lotion

ACTIVE INGREDIENT: Salicylic Acid 2%
INACTIVE INGREDIENTS: water, polyethylene, PPG-15 stearyl ether, glycerin, stearyl alcohol, cetyl betaine, distearyldimonium chloride, sodium lauryl sulfate, cetyl alcohol, alcohol, steareth-21, sodium chloride, behenyl alcohol, steareth-2, synthetic wax, fragrance, lavandula stoechas extract, helichrysum italicum extract, cistus monspeliensis extract, xanthan gum, lauryl alcohol, mica, disodium EDTA, BHT, ferric ferrocyanide, titanium dioxide, FD&C blue no. 1

### Production Example 108: Salicyclic Acid Lotion

ACTIVE INGREDIENT: Salicylic Acid 2%
INACTIVE INGREDIENTS: water, polyethylene, PPG-15 stearyl ether, glycerin, stearyl alcohol, cetyl betaine, distearyldimonium chloride, sodium lauryl sulfate, cetyl alcohol, alcohol, steareth-21, sodium chloride, behenyl alcohol, synthetic wax, steareth-2, fragrance, lavandula stoechas extract, helichrysum italicum extract, cistus monspeliensis extract, xanthan gum, dimethyl palmitamine, lauryl alcohol, mica, isopropyl alcohol, disodium EDTA, BHT, magnesium nitrate, methylchloroisothiazolinone, magnesium chloride, methylisothiazolinone, sodium sulfate, ferric ferrocyanide, titanium dioxide, FD&C blue no. 1

### Production Example 109: Salicyclic Acid Lotion

ACTIVE INGREDIENT: Salicylic Acid 2%
INACTIVE INGREDIENTS: water, sodium cocoyl isethionate, cetearyl alcohol, glycerin, sodium laureth sulfate, sodium cocoamphoacetate, cocamidopropyl betaine, sodium hydroxide, acrylates/C10-30 alkyl acrylate crosspolymer, fragrance, disodium EDTA, Lavendula stoechas extract, Helichrysum italicum extract, Cistus monspeliensis extract.

### Production Example 110: Salicyclic Acid Solution

ACTIVE INGREDIENT: Salicylic Acid 2%
INACTIVE INGREDIENTS: water, alcohol denat., glycerin, isoceteth-20, sorbitol, sodium hydroxide, fragrance, lavandula stoechas extract, helichrysum italicum extract, cistus monspeliensis extract, disodium EDTA.

### Production Example 111: Salicyclic Acid Cream

ACTIVE INGREDIENT: Salicylic Acid 2%
INACTIVE INGREDIENTS: water, PPG-15 stearyl ether, hydrated silica, glycerin, stearyl alcohol, cetyl betaine, distearyldimonium chloride, sodium lauryl sulfate, cetyl alcohol, alcohol denat, steareth-21, sodium chloride, behenyl alcohol, synthetic wax, steareth-2, fragrance, Lavandula stoechas extract, Helichrysum italicum extract, Cistus monspeliensis extract, xanthan gum, dimethyl palmitamine, lauryl alcohol, mica, isopropyl alcohol, disodium EDTA, BHT, magnesium nitrate, methylchloroisothiazolinone, magnesium chloride, methylisothiazolinone, sodium sulfate, ferric ferrocyanide, titanium dioxide, FD&C blue no. 1

### Production Example 112: Salicyclic Acid Lotion

ACTIVE INGREDIENT: Salicylic Acid 2%
INACTIVE INGREDIENTS: water, polyethylene, PPG-15 stearyl ether, glycerin, stearyl alcohol, cetyl betaine, distearyldimonium chloride, sodium lauryl sulfate, cetyl alcohol, alcohol, steareth-21, sodium chloride, behenyl alcohol, synthetic wax, steareth-2, fragrance, Lavandula stoechas extract, Helichrysum italicum extract, Cistus monspeliensis extract, xanthan gum, dimethyl palmitamine, lauryl alcohol, mica, isopropyl alcohol, disodium EDTA, BHT, magnesium nitrate, methylchloroisothiazolinone, magnesium chloride, methylisothiazolinone, sodium sulfate, ferric ferrocyanide, titanium dioxide, FD&C blue no. 1.

### Production Example 113: Salicyclic Acid Gel

ACTIVE INGREDIENT: Salicylic Acid 2%
INACTIVE INGREDIENTS: Water, Alcohol Denat., Sodium Gluconate, Propylene Glycol, Hydroxyethylcellulose, Isoceteth-20, Dimethylacrylamide/Acrylic Acid/Polystyrene Ethyl Methacrylate Copolymer, Sodium Hydroxide, Disodium EDTA, Phenoxyethanol, Sodium Benzoate, Oryza Saliva (Rice) Extract.

### Production Example 114: Salicyclic Acid Gel

ACTIVE INGREDIENT: Salicylic Acid 2%
INACTIVE INGREDIENTS: Water, Alcohol Denat., Polysorbate 20, Sorbitol, Hydroxyethylcellulose, Glycerin, Sodium Hydroxide, Fragrance, Lavandula Stoechas Extract, Helichrysum Italicum Extract, Cistus Monspeliensis Extract, Menthol, Disodium EDTA.

### Production Example 115: Salicyclic Acid Lotion

ACTIVE INGREDIENT: Salicylic Acid 2%
INACTIVE INGREDIENTS: water, butylene glycol, octyldodecanol, steareth-2, cetyl alcohol, steareth-21, dimethicone, polyacrylamide, glycerin, C13-14 isoparaffin, sodium hydroxide, xanthan gum, magnesium aluminum silicate, laureth-7, fragrance, Lavandula stoechas extract, Helichrysum italicum extract, Cistus monspeliensis extract, titanium dioxide.

### Production Example 116: Salicyclic Acid Gel

ACTIVE INGREDIENT: Salicylic Acid 2%
INACTIVE INGREDIENTS: Water, Cocamidopropyl Hydroxysultaine, Sodium Methyl Cocoyl Taurate, Sodium Chloride, Glycerin, Sodium Hydrolyzed Potato Starch Dodecenylsuccinate, Fragrance, Sodium Hydroxide, PEG-120 Methyl Glucose Dioleate, Panthenol, C12-15 Alkyl Lactate, Disodium EDTA, Cocamidopropyl PG-Dimonium Chloride Phosphate, Benzalkonium Chloride.

### Production Example 117: Salicyclic Acid Gel

ACTIVE INGREDIENT: Salicylic Acid 2%
INACTIVE INGREDIENTS: Water, Cocamidopropyl Hydroxysultaine, Sodium Methyl Cocoyl Taurate, Sodium Chloride, Glycerin, Sodium Hydrolyzed Potato Starch Dodecenylsuccinate, Fragrance, Sodium Hydroxide, PEG-120 Methyl Glucose Dioleate, Panthenol, C12-15 Alkyl Lactate, Disodium EDTA, Cocamidopropyl PG-Dimonium Chloride Phosphate, Benzalkonium Chloride.

### Production Example 118: Salicyclic Acid Gel

ACTIVE INGREDIENT: Salicylic Acid 2%
INACTIVE INGREDIENTS: Water, Sodium C14-16 Olefin Sulfonate, Cocamidopropyl Hydroxysultaine, Sorbitol Cellulose, Sodium Hydrolyzed Potato Starch Dodecenylsuccinate, Acrylates Crosspolymer-4, Panthenol, Sodium Hydroxide, Fragrance, Microcrystalline Wax, C12-15 Alkyl Lactate, Benzalkonium Chloride, Disodium EDTA, Cocamidopropyl PG-Dimonium Chloride Phosphate, Talc, Yellow 5 Lake, Red 30.

### Production Example 119: Salicyclic Acid Aerosol, Foam

ACTIVE INGREDIENT: Salicylic Acid 2%
INACTIVE INGREDIENTS: Water, Glycerin, Cocamidopropyl Betaine, Isobutane, Methyl Gluceth-20, Decyl Glucoside, Sodium Chloride, Potassium Acrylates Copolymer, Sodium Hydroxide, Fragrance, Panthenol, PEG-120 Methyl Glucose Dioleate, Menthol, Disodium EDTA.

### Production Example 120: Salicyclic Acid Liquid

ACTIVE INGREDIENT: Salicylic Acid 2%
INACTIVE INGREDIENTS: water, cetyl alcohol, PPG-15 stearyl ether, cellulose, glycerin, Polysorbate 60, steareth-21, potassium cetyl phosphate, xanthan gum, fragrance, disodium EDTA, menthyl lactate, sodium hydroxide.

### Production Example 121: Salicyclic Acid Cream

ACTIVE INGREDIENT: Salicylic Acid 1%
INACTIVE INGREDIENTS: Water, C12-15 Alkyl Benzoate, Cetearyl Alcohol, Glycerin, Dimethicone, PPG-10 Cetyl Ether, Arachidyl Alcohol, Methyl Gluceth-20, Glycine Soja (Soybean) Seed Extract, Behenyl Alcohol, Polyacrylamide, Isoceteth-20, Cetearyl Glucoside, Phenyl Trimethicone, Phenoxyethanol, Arachidyl Glucoside, C13-14 Isoparaffin, Benzyl Alcohol, Ethylene/Acrylic Acid Copolymer, Disodium EDTA, Fragrance, Ammonium Hydroxide, Methylparaben, Laureth-7, BHT, Ascorbyl Palmitate, Benzalkonium Chloride, Propylparaben, 20, Ethylparaben, Retinol, Lactobacillus/Soybean Seed Ferment Extract.

### Production Example 122: Salicyclic Acid Lotion

ACTIVE INGREDIENT: Salicylic Acid 2%
INACTIVE INGREDIENTS: water, cetyl alcohol, polyethylene, PPG-15 stearyl ether, polysorbate 60, steareth-21, potassium cetyl phosphate, xanthan gum, steareth-2, C12-15 alkyl lactate, cocamidopropyl PG-dimonium chloride phosphate, fragrance, sodium hydroxide, cetyl lactate, menthyl lactate, disodium EDTA, benzalkonium chloride, citric acid, titanium dioxide

### Production Example 123: Salicyclic Acid Gel

ACTIVE INGREDIENT: Salicylic Acid 2%
INACTIVE INGREDIENTS: water, cetyl alcohol, cellulose, PPG-15 stearyl ether, glycerin, polysorbate 60, steareth-21, potassium cetyl phosphate, xanthan gum, fragrance, disodium EDTA, menthyl lactate, sodium hydroxide, agar, iron oxides, mica, titanium dioxide, red 30.

### Production Example 124: Salicyclic Acid Gel

ACTIVE INGREDIENT: Salicylic Acid 2%
INACTIVE INGREDIENTS: water, cetyl alcohol, cellulose, PPG-15 stearyl ether, glycerin, polysorbate 60, steareth-21, potassium cetyl phosphate, xanthan gum, fragrance, disodium EDTA, menthyl lactate, sodium hydroxide, agar, iron oxides, mica, titanium dioxide, red 30.

### Production Example 125: Salicyclic Acid Strip

ACTIVE INGREDIENT: Salicylic Acid 2%
INACTIVE INGREDIENTS: Polyvinyl Alcohol, Cellulose, Sodium C14-16 Olefin Sulfonate, Cocamidopropyl Hydroxysultaine, Glycerin, Water, Glycolic Acid, Menthol, Fragrance, Sodium Hydroxide, Menthyl Lactate, Disodium EDTA, Citric Acid, Yellow 5, Red 40.

### Production Example 126: Salicyclic Acid Spray

ACTIVE INGREDIENT: Salicylic Acid 0.5%
INACTIVE INGREDIENTS: Water, Alcohol Denat., Butylene Glycol, PPG-5-Ceteth-20, C12-15 Alkyl Lactate, Sodium Citrate, Cocamidopropyl PG-Dimonium Chloride Phosphate, PEG/PPG-20/6 Dimethicone, Cetyl Lactate, Fragrance, Benzalkonium Chloride, Disodium EDTA, Glycerin, Camellia Oleifera Leaf Extract, Cucumis Sativus (Cucumber) Fruit Extract.

### Production Example 127: Salicyclic Acid Suspension

ACTIVE INGREDIENT: Salicylic Acid 2%
INACTIVE INGREDIENTS: Water, Sodium C14-16 Olefin Sulfonate, Glycerin, Cocamidopropyl Betaine, Jojoba Esters, Acrylates Copolymer, Sodium Chloride, Fragrance, Sodium Hydroxide, Disodium EDTA, Camellia Oleifera Leaf Extract, Butylene Glycol, Chamomilla Recutita (Matricaria) Flower Extract, Citric Acid, Yellow 6, Yellow 5, Red 40, Red 33, Blue 1.

### Production Example 128: Salicyclic Acid Solution

ACTIVE INGREDIENT: Salicylic Acid 2%
INACTIVE INGREDIENTS: water, sodium C14-16 olefin sulfonate, cocamidopropyl betaine, disodium cocoamphodiacetate, glycerin, sodium methyl cocoyl taurate, sodium chloride, propylene glycol, linoleamidropropyl PG-dimonium chloride phosphate, fragrance, citric acid, aloe barbadensis leaf extract, anthemis nobilis flower extract, chamomilla recutita (matricaria) flower extract, yellow 5, red 33.

### Production Example 129: Salicyclic Acid Suspension

ACTIVE INGREDIENT: Salicylic Acid 2%
INACTIVE INGREDIENTS: water, sodium C14-16 olefin sulfonate, glycerin, cocamidopropyl betaine, acrylates copolymer, sodium chloride, polysorbate 20, fragrance, sodium hydroxide, benzalkonium chloride, C12-15 alkyl lactate, disodium EDTA, polyquaternium-7, cocamidopropyl PG-dimonium chloride phosphate, sodium benzotriazolyl butylphenol sulfonate, agar, carrageenan, ascorbyl palmitate, propylene glycol, camellia oleifera leaf extract, butylene glycol, chamomilla recutita (matricaria) flower extract, citrus grandis (grapefruit) fruit extract, mica, titanium dioxide, iron oxides, red 30 lake, red 40, red 30, violet 2.

### Production Example 130: Salicyclic Acid Liquid

ACTIVE INGREDIENT: Salicylic Acid 2%
INACTIVE INGREDIENTS: Water, Alcohol Denat., Hamamelis Virginiana (Witch Hazel) Water, Butylene Glycol, Glycereth-7, Methyl Gluceth-20, Alcohol, Fragrance, Propylene Glycol, Benzophenone-4, Aloe Barbadensis Leaf Extract, Chamomilla Recutita (Matricaria) Flower Extract, Blue 1.

### Production Example 131: Salicyclic Acid Gel

ACTIVE INGREDIENT: Salicylic Acid 0.5%
INACTIVE INGREDIENTS: Water, Sodium C14-16 Olefin Sulfonate, Cocamidopropyl Hydroxysultaine, Cellulose Sorbitol, Acrylates Crosspolymer-4, Sodium Hydrolyzed Potato Starch Dodecenylsuccinate, Polysorbate 20, Glycolic Acid, Fragrance, Microcrystalline Wax, Sodium Hydroxide, Menthol, Disodium EDTA, Ferric Ferrocyanide, Green 3.

### Production Example 132: Salicyclic Acid Cream

ACTIVE INGREDIENT: Salicylic Acid 0.5%
INACTIVE INGREDIENTS: Water, Glycerin, Kaolin, Bentonite, Sodium Methyl Cocoyl Taurate, Trideceth-9, PEG-5 Ethylhexanoate, Xanthan Gum, Glycolic Acid, Citric Acid, Menthyl Lactate, Panthenol, Sodium Hydroxide, Fragrance, Sodium Citrate, Disodium EDTA, Titanium Dioxide, Green 3.

### Production Example 133: Salicyclic Acid Aerosol, Foam

ACTIVE INGREDIENT: Salicylic Acid 0.5%
INACTIVE INGREDIENTS: Water, Glycerin, Cocamidopropyl Betaine, Isobutane, Methyl Gluceth-20, Decyl Glucoside, Sodium Chloride, Potassium Acrylates Copolymer, Glycolic Acid, Fragrance, PEG-120 Methyl Glucose Dioleate, Sodium Hydroxide, Menthol Disodium EDTA.

### Production Example 134: Salicyclic Acid Lotion

ACTIVE INGREDIENT: Salicylic Acid 1%
INACTIVE INGREDIENTS: Water, cetyl alcohol, glycerin, sucrose cocoate, hydroxypropyl starch phosphate, coco-glucoside, potassium cetyl phosphate, xanthan gum, sodium benzoate, fragrance, menthol, citric acid, sodium phytate, butylene glycol, cedrus atlantica bark extract, portulaca oleracea extract.

### Production Example 135: Salicyclic Acid Lotion

ACTIVE INGREDIENT: Salicylic Acid 1%
INACTIVE INGREDIENTS: Water, Glycerin, Decyl Glucoside, Jojoba Esters, Propanediol, Polyglyceryl-10 Oleate, Xanthan Gum, Hydroxypropyl Starch Phosphate, Coco-Glucoside, Glyceryl Oleate, Sodium Benzoate, Citric Acid, Fragrance, Sodium Phytate, Butylene Glycol, Cedrus Atlantica Bark Extract, Portulaca Oleracea Extract.

### Production Example 136: Salicyclic Acid Lotion

ACTIVE INGREDIENT: Salicylic Acid 1%
INACTIVE INGREDIENTS: water, hydrated silica, glycerin, dicaprylyl carbonate, cetearyl alcohol, magnesium aluminum silicate, hydroxypropyl starch phosphate, coca-glucoside, benzyl alcohol, sodium benzoate, xanthan gum, cetearyl glucoside, sodium hydroxide, fragrance, sodium phytate, butylene glycol, cedrus atlantica bark extract, portulaca oleracea extract.

### Production Example 137: Salicyclic Acid Lotion

ACTIVE INGREDIENT: Salicylic Acid 0.5%
INACTIVE INGREDIENTS: Water, Dimethicone, Dicaprylyl Ether, Glycerin, C12-15 Alkyl Benzoate, Phenyl Trimethicone, Cetearyl Alcohol, Ceteth-10 Phosphate, Dicetyl Phosphate, Methyl Gluceth-20, Magnesium Aluminum Silicate, Steareth-20, Steareth-2, Xanthan Gum, Sodium C14-16 Olefin Sulfonate, Fragrance, Sodium Hydroxide, C12-15 Alkyl Lactate, Cetyl Lactate, Benzalkonium Chloride, Cocamidopropyl PG-Dimonium Chloride Phosphate, Propylene Glycol, Tetrasodium Glutamate Diacetate, Citrus Grandis (Grapefruit) Fruit Extract, Titanium Dioxide, Iron Oxides

### Production Example 138: Salicyclic Acid Lotion

ACTIVE INGREDIENT: Salicylic Acid 0.5%
INACTIVE INGREDIENTS: Water, Dimethicone, Dicaprylyl Ether, Glycerin, C12-15 Alkyl Benzoate, Phenyl Trimethicone, Cetearyl Alcohol, Ceteth-10 Phosphate, Dicetyl Phosphate, Methyl Gluceth-20, Magnesium Aluminum Silicate, Steareth-20, Steareth-2, Xanthan Gum, Sodium C14-16 Olefin Sulfonate, Fragrance, Sodium Hydroxide, C12-15 Alkyl Lactate, Cetyl Lactate, Benzalkonium Chloride, Cocamidopropyl PG-Dimonium Chloride Phosphate, Propylene Glycol, Tetrasodium Glutamate Diacetate, Citrus Grandis (Grapefruit) Fruit Extract, iron Oxides, Titanium Dioxide

### Production Example 139: Salicyclic Acid Lotion

ACTIVE INGREDIENT: Salicylic Acid 0.5%
INACTIVE INGREDIENTS: water, dicaprylyl ether, cetearyl alcohol, glycerin, neopentyl glycol diethylhexanoate, dimethicone, methyl gluceth-20, aluminum starch octenylsuccinate, ceteth-10 phosphate, dicetyl phosphate, steareth-20, steareth-2, neopentyl glycol diisostearate, sodium benzoate, fragrance, sodium hydroxide, xanthan gum, magnesium aluminum silicate, disodium EDTA, BHT, propylene glycol, citrus grandis (grapefruit) fruit extract, titanium dioxide

### Production Example 140: Salicyclic Acid Cream

ACTIVE INGREDIENT: Salicylic Acid 2%
INACTIVE INGREDIENTS: Water, Cetyl Alcohol, PPG-15 Stearyl Ether, Glycolic Acid, Glycerin, Polysorbate 60, C12-15 Alkyl Lactate, Cetyl Lactate, Potassium Cetyl Phosphate, Sodium Hydroxide, Fragrance, Cocamidopropyl PG-Dimonium Chloride Phosphate, Xanthan Gum, Disodium EDTA, Menthol, Benzalkonium Chloride, Panthenol, Butylene Glycol, Camellia Oleifera Leaf Extract, Cucumus Sativus (Cucumber) Fruit Extract, Green 5, Yellow 5, Blue 1

### Production Example 141: Salicyclic Acid Cloth

ACTIVE INGREDIENT: Salicylic Acid 2%
INACTIVE INGREDIENTS: Water, Alcohol Denat., Butylene Glycol, PPG-5-Ceteth-20, C12-15 Alkyl Lactate, Sodium Citrate, Fragrance, Cocamidopropyl PG-Dimonium Chloride Phosphate, PEG/PPG-20/6 Dimethicone, Sodium Hydroxide, Benzalkonium Chloride, Cetyl Lactate, Disodium EDTA

### Production Example 142: Salicyclic Acid Gel

ACTIVE INGREDIENT: Salicylic Acid 2%
INACTIVE INGREDIENTS: Water, Cetyl Alcohol, PPG-15 Stearyl Ether, Microcrystalline Cellulose, Glycolic Acid, Glycerin, Polysorbate 60, Steareth-21, C12-15 Alkyl Lactate, Cetyl Lactate, Potassium Cetyl Phosphate, Microcrystalline Wax, Sodium Hydroxide, Xanthan Gum, Cocamidopropyl PG-Dimonium Chloride Phosphate, Fragrance, Disodium EDTA, Menthol, Benzalkonium Chloride, Camellia Sinensis Leaf Extract, Sodium Benzotriazolyl Butylphenol Sulfonate, Butylene Glycol, Cucumis Sativus (Cucumber) Fruit Extract, Chromium Hydroxide Green, Green 5, Yellow 5, Blue 1

### Production Example 143: Salicyclic Acid Liquid

ACTIVE INGREDIENT: Salicylic Acid 2%
INACTIVE INGREDIENTS: water, alcohol denat., butylene glycol, PPG-5-ceteth-20, C12-15 alkyl lactate, fragrance, sodium citrate, cocamidopropyl PG-dimonium chloride phosphate, PEG/PPG-20/6 dimethicone, sodium hydroxide, benzalkonium chloride, cetyl lactate, sodium benzotriazolyl butylphenol sulfonate, disodium EDTA, propylene glycol, cucumis sativus (cucumber) fruit extract, camellia oleifera leaf extract, blue 1, green 5, yellow 5

### Production Example 144: Salicyclic Acid Liquid

ACTIVE INGREDIENT: Salicylic Acid 2%
INACTIVE INGREDIENTS: Water, Alcohol Denat., Butylene Glycol, PPG-5-Ceteth-20, C12-15 Alkyl Lactate, Fragrance, Sodium Citrate, Cocamidopropyl PG-Dimonium Chloride Phosphate, PEG/PPG-20/6 Dimethicone, Sodium Hydroxide, Benzalkonium Chloride, Cetyl Lactate, Sodium Benzotriazolyl Butylphenol Sulfonate, Disodium EDTA, Camellia Sinensis Leaf Extract, Glycerin, Cucumis Sativus (Cucumber) Fruit Extract, Yellow 5, Blue 1, Green 5

### Production Example 145: Salicyclic Acid Gel

ACTIVE INGREDIENT: Salicylic Acid 2%
INACTIVE INGREDIENTS: Water, Sodium C14-16 Olefin Sulfonate, Cocamidopropyl Betaine, Sodium Chloride, PEG-80 Sorbitan Laurate, C12-15 Alkyl Lactate, Benzalkonium Chloride, Disodium EDTA, Fragrance, Cocamidopropyl PG-Dimonium Chloride Phosphate, Propylene Glycol, Aloe Barbadensis Leaf Extract, Anthemis Nobilis Flower Extract, Chamomilla Recutita (Matricaria) Flower Extract, Sodium Hydroxide, Citric Acid, Yellow 5, Red 40

### Production Example 146: Salicyclic Acid Gel

ACTIVE INGREDIENT: Salicylic Acid 2%
INACTIVE INGREDIENTS: Water, Sodium C14-16 Olefin Sulfonate, Cocamidopropyl Betaine, Sodium Chloride, PEG-80 Sorbitan Laurate, C12-15 Alkyl Lactate, Benzalkonium Chloride, Disodium EDTA, Fragrance, Cocamidopropyl PG-Dimonium Chloride Phosphate, Propylene Glycol, Aloe Barbadensis Leaf Extract, Anthemis Nobilis Flower Extract, Chamomilla Recutita (Matricaria) Flower Extract, Sodium Hydroxide, Citric Acid, Yellow 5, Red 40

### Production Example 147: Salicyclic Acid Lotion

ACTIVE INGREDIENT: Salicylic Acid 2%
INACTIVE INGREDIENTS: Water, Sodium C14-16 Olefin Sulfonate, Cocamidopropyl Betaine, Sodium Chloride, PEG-80 Sorbitan Laurate, Benzalkonium Chloride, Disodium EDTA, C12-15 Alkyl Lactate, Fragrance, Cocamidopropyl PG-Dimonium Chloride Phosphate, Propylene Glycol, Aloe Barbadensis Leaf Extract, Anthemis Nobilis Flower Extract, Chamomilla Recutita (Matricaria) Flower Extract, Citric Acid, Sodium Hydroxide, Yellow 5, Red 40

### Production Example 148: Salicyclic Acid Cream

ACTIVE INGREDIENT: Salicylic Acid 2%
INACTIVE INGREDIENTS: Water, Cetyl Alcohol, PPG-15 Stearyl Ether, Glycolic Acid, Glycerin, Steareth-21, Polysorbate 60, C12-15 Alkyl Lactate, Cetyl Lactate, Potassium Cetyl Phosphate, Sodium Hydroxide, Cocamidopropyl PG-Dimonium Chloride Phosphate, Xanthan Gum, Fragrance, Disodium EDTA, Menthol, Benzalkonium Chloride, Panthenol

### Production Example 149: Salicyclic Acid Lotion

ACTIVE INGREDIENT: Salicylic Acid 2%
INACTIVE INGREDIENTS: water, cetyl alcohol, PPG-15 stearyl ether, glycolic acid, steareth-21, glycerin, polysorbate 60, C12-15 alkyl lactate, cetyl lactate, potassium cetyl phosphate, sodium hydroxide, cocamidopropyl PG-dimonium chloride phosphate, fragrance, xanthan gum, disodium EDTA, menthol, benzalkonium chloride, panthenol, sodium benzotriazolyl butylphenol sulfonate, ascorbyl palmitate, propylene glycol, citrus grandis (grapefruit) fruit extract, red 40, yellow 6

### Production Example 150: Salicyclic Acid Liquid

ACTIVE INGREDIENT: Salicylic Acid 2%
INACTIVE INGREDIENTS; water, sodium C14-16 olefin sulfonate, cocamidopropyl betaine, sodium chloride, PEG-120 methyl glucose dioleate, polysorbate 20, linoleamidopropyl PG-dimonium chloride phosphate, propylene glycol, PEG-80 sorbitan laurate, fragrance, disodium EDTA, benzalkonium chloride, C12-15 alkyl lactate, polyquaternium-7, sodium benzotriazolyl butylphenol sulfonate, cocamidopropyl PG-dimonium chloride phosphate, ascorbyl palmitate, aloe barbadensis leaf extract, anthemis nobilis flower extract, chamomilla recutita (matricaria) flower extract, citrus grandis (grapefruit) fruit extract, citric acid, sodium hydroxide, red 40, violet 2

### Production Example 151: Salicyclic Acid Lotion

ACTIVE INGREDIENT: Salicylic Acid 2%
INACTIVE INGREDIENTS: water, cetyl alcohol, PPG-15 stearyl ether, glycolic acid, steareth-21, glycerin, polysorbate 60, C12-15 alkyl lactate, cetyl lactate, potassium cetyl phosphate, sodium hydroxide, xanthan gum, fragrance, cocamidopropyl PG-dimonium chloride phosphate, disodium EDTA, menthol, benzalkonium chloride, sodium benzotriazolyl butylphenol sulfonate, panthenol, propylene glycol, aloe barbadensis leaf extract, chamomilla recutita (matricaria) flower extract, yellow 5, blue 1

### Production Example 152: Salicyclic Acid Solution

ACTIVE INGREDIENT: Salicylic Acid 2%
INACTIVE INGREDIENTS: Water, Sodium C14-16 Olefin Sulfonate, Cocamidopropyl Betaine, PEG-120 Methyl Glucose Dioleate, Sodium Chloride, Linoleamidopropyl PG-Dimonium Chloride Phosphate, Polysorbate 20, Propylene Glycol, Fragrance, PEG-80 Sorbitan Laurate, Disodium EDTA, Sodium Glycolate, Sodium Hydroxide, C12-15 Alkyl Lactate, Benzalkonium Chloride, Sodium Benzotriazolyl Butylphenol Sulfonate, Cocamidopropyl PG-Dimonium Chloride Phosphate, 3-Ditnethylaminopropylamine, Polyquaternium-7, Ethanol, Tocopheryl Acetate, Tetrasodium EDTA, Aloe Barbadensis Leaf Extract, Chamomilla Recutita (Matricaria) Flower Extract, Citric Acid, Yellow 5, Blue 1

### Production Example 153: Salicyclic Acid Cream

ACTIVE INGREDIENT: Salicylic Acid 2%
INACTIVE INGREDIENTS: Water, Cetyl Alcohol, PPG-15 Stearyl Ether, Glycolic Acid, Glycerin, Polysorbate 60, Steareth-21, C12-15 Alkyl Lactate, Cetyl Lactate, Potassium Cetyl Phosphate, Sodium Hydroxide, Cocamidopropyl PG-Dimonium Chloride Phosphate, Fragrance, Xanthan Gum, Disodium EDTA, Menthol, Benzalkonium Chloride, Panthenol, Camellia Sinensis Leaf Extract, Butylene Glycol, Cucumus Sativus (Cucumber) Fruit Extract, Green 5, Yellow 5, Blue 1

### Production Example 154: Salicyclic Acid Gel

ACTIVE INGREDIENT: Salicylic Acid 2%
INACTIVE INGREDIENTS: water, cetyl alcohol, PPG-15 stearyl ether, cellulose, glycerin, Polysorbate 60, steareth-21, C12-15 alkyl lactate, glycolic acid, cetyl lactate, potassium cetyl phosphate, sodium hydroxide, xanthan gum, cocamedopropyl PG-dimonium chloride phosphate, disodium EDTA, fragrance, menthol, Benzalkonium chloride, agar, BHT, sodium benzotriazolyl, butylphenone sulfonate, iron oxides, mica, yellow 5, titanium dioxide, red 30, red 40

### Production Example 155: Salicyclic Acid Gel

ACTIVE INGREDIENT: Salicylic Acid 2%
INACTIVE INGREDIENTS: water, sodium C14-16 olefin sulfonate, glycerin, cocamidopropyl betaine, microcrystalline cellulose, acrylates copolymer, cellulose, Polysorbate 20, fragrance, sodium hydroxide, ascorbyl palmitate, C12-15 alkyl lactate, benzalkonium chloride, disodium EDTA, polyquaternuim-7, cocamidopropyl PG-dimonium chloride phosphate, sodium benzotriazolyl butylphenol sulfonate, propylene glycol, citrus grandis (grape fruit) fruit extract, carrageenan, agar, mica, titanium dioxide, red 30, red 40, violet 2.

### Production Example 156: Salicyclic Acid Swab

ACTIVE INGREDIENT: Salicylic Acid 2%
INACTIVE INGREDIENTS: Water, Alcohol Denat. (38%), Butylene Glycol, PPG-5-Ceteth-20, C12-15 Alkyl Lactate, Cetyl Lactate, Cocamidopropyl PG-Dimonium Chloride Phosphate, PEG/PPG-20/6 Dimethicone, Benzalkonium Chloride, Sodium Citrate, Disodium EDTA, Sodium Hydroxide, Fragrance

### Production Example 157: Salicyclic Acid Lotion

ACTIVE INGREDIENT: Salicylic Acid 2%
INACTIVE INGREDIENTS: Alcohol Denat. (38%), Hamamelis Virginiana (Witch Hazel) Water, Water, Polyquaternium-37, Glycerin, Glycolic Acid, Butylene Glycol, Cyclohexasiloxane, C12-15 Alkyl Lactate, Isodecyl Laurate, Cyclopentasiloxane, Cetyl Lactate, Polysorbate 20, Portulaca Oleracea Extract, Sodium Benzotriazolyl Butylphenol Sulfonate, Cocamidopropyl PG-Dimonium Chloride Phosphate, Sodium Hydroxide, Sodium Chloride, Bisabolol, Benzalkonium Chloride, Yellow 6, Red 40, Fragrance

### Production Example 158: Salicyclic Acid Solution

ACTIVE INGREDIENT: Salicylic Acid 2%
INACTIVE INGREDIENTS: water, alcohol denat., butylene glycol, PPG-5-ceteth-20, C12-15 alkyl lactate, sodium citrate, fragrance, cocamidopropyl PG-dimonium chloride phosphate, PEG/PPG-20/6 dimethicone, cetyl lactate, benzalkonium chloride, sodium benzotriazolyl butylphenol sulfonate, disodium EDTA, sodium hydroxide, glycolic acid, yellow 6, red 40

### Production Example 159: Salicyclic Acid Kit

ACTIVE INGREDIENT: Salicylic Acid 2%
INACTIVE INGREDIENTS: Alcohol Denat, Hamamelis Virginiana (Witch Hazel) Water, Water, Polyquaternium-37, Glycerin, Glycolic Acid, Butylene Glycol, Cyclohexasiloxane, C12-15 Alkyl Lactate, Isodecyl Laurate, Cyclopentasiloxane, Cetyl Lactate, Polysorbate 20, Portulaca Oleracea Extract, Sodium Benzotriazolyl Butylphenol Sulfonate, Cocamidopropyl PG-Dimonium Chloride Phosphate, Sodium Hydroxide, Sodium Chloride, Bisabolol, Benzalkonium Chloride, Yellow 6, Red 40, Fragrance.

### Production Example 160: Salicyclic Acid Lotion

ACTIVE INGREDIENT: Salicylic Acid 2%
INACTIVE INGREDIENTS: Water, C12-15 Alkyl Benzoate, Butylene Glycol, Cetyl Alcohol, Dimethicone, Dimethicone PEG-8 Beeswax, Glyceryl Stearate, PEG-100 Stearate, Potassium Cetyl Phosphate, Capryloyl Glycine, Hydrogenated Palm Glycerides, Acrylates/C10-30 Alkyl Acrylate Crosspolymer, Xanthan Gum, Sarcosine, Sodium Hydroxide, Propylene Glycol, Fragrance, Tocopheryl Acetate, Cinnamomum Zeylanicum Bark Extract, Bisabolol, Allantoin, Cedrus Atlantica Bark Extract, Portulaca Oleracea Extract, Citric Acid

### Production Example 161: Salicyclic Acid Gel

ACTIVE INGREDIENT: Salicylic Acid 2%
INACTIVE INGREDIENTS: water, alcohol denat., hamamelis virginiana (witch hazel) water, polyacrylamide, glycerin, C13-14 isoparaffin, cyclopentasiloxane, C12-15 alkyl lactate, alcohol, laureth-7, phenoxyethanol, cetyl lactate, PPG-2 isoceteth-20 acetate, cocamidopropyl PG-dimonium chloride phosphate, polysorbate 20, phenethyl dimethicone, fragrance, dehydroxanthan gum, propylene glycol, benzalkonium chloride, ammonium hydroxide, tetrasodium EDTA, butylene glycol, capryloyl glycine, sarcosine, cinnamomum zeylanicum bark extract, cedrus atlantica bark extract, portulaca oleracea extract

### Production Example 162: Salicyclic Acid Gel

ACTIVE INGREDIENT: Salicylic Acid 2%
INACTIVE INGREDIENTS: Water, Sodium C14-16 Olefin Sulfonate, Glycerin, Cocamidopropyl Betaine, Microcrystalline Cellulose, Acrylates Copolymer, Fragrance, Polysorbate 20, Sodium Hydroxide, C12-15 Alkyl Lactate, Benzalkonium Chloride, Polyquaternium-7, Disodium EDTA, Menthol, Cocamidopropyl PG-Dimonium Chloride Phosphate, Sodium Benzotriazolyl Butylphenol Sulfonate, Carrageenan, Agar, Ascorbyl Palmitate, Iron Oxides, Yellow 5, Red 40, Mica, Red 30, Titanium Dioxide

### Production Example 163: Salicyclic Acid Cream

ACTIVE INGREDIENT: Salicylic Acid 0.5%
INACTIVE INGREDIENTS: Water, cyclopentasiloxane, dimethicone, kaolin, cyclomethicone, VP/hexadecene copolymer, synthetic wax, cetyl PEG/PPG-10/1 dimethicone, trisiloxane, BIS-PEG/PPG-14/14 dimethicone, cyclohexasiloxane, butylene glycol, silica, phenoxyethanol, boron nitride, polysilicone-11, quaternium-90 bentonite, sodium chloride, magnesium sulfate, propylene glycol, methylparaben, farnesol, chamomilla recutita (matricaria) flower extract, propylene carbonate, triethoxycaprylylsilane, propylparaben, aloe barbadensis leaf juice, polymethyl methacrylate, tocopheryl acetate, cocamidopropyl PG-dimonium chloride phosphate, cetyl lactate, C12-15 alkyl lactate, benzalkonium chloride, retinyl palmitate, pantothenic acid, ascorbic acid May Contain: titanium dioxide, iron oxides

### Production Example 164: Salicyclic Acid Cream

ACTIVE INGREDIENT: Salicylic Acid 0.5%
INACTIVE INGREDIENTS: Water, cyclopentasiloxane, dimethicone, kaolin, cyclomethicone, VP/hexadecene copolymer, synthetic wax, cetyl PEG/PPG-10/1 dimethicone, trisiloxane, BIS-PEG/PPG-14/14 dimethicone, cyclohexasiloxane, butylene glycol, silica, phenoxyethanol, boron nitride, polysilicone-11, quaternium-90 bentonite, sodium chloride, magnesium sulfate, propylene glycol, methylparaben, farnesol, chamomilla recutita (matricaria) flower extract, propylene carbonate, triethoxycaprylylsilane, propylparaben, aloe barbadensis leaf juice, polymethyl methacrylate, tocopheryl acetate, cocamidopropyl PG-dimonium chloride phosphate, cetyl lactate, C12-15 alkyl lactate, benzalkonium chloride, retinyl palmitate, pantothenic acid, ascorbic acid May Contain: titanium dioxide, iron oxides

### Production Example 165: Salicyclic Acid Cream

ACTIVE INGREDIENT: Salicylic Acid 0.5%
INACTIVE INGREDIENTS: Water, cyclopentasiloxane, dimethicone, kaolin, cyclomethicone, VP/hexadecene copolymer, synthetic wax, cetyl PEG/PPG-10/1 dimethicone, trisiloxane, BIS-PEG/PPG-14/14 dimethicone, cyclohexasiloxane, butylene glycol, silica, phenoxyethanol, boron nitride, polysilicone-11, quaternium-90 bentonite, sodium chloride, magnesium sulfate, propylene glycol, methylparaben, farnesol, chamomilla recutita (matricaria) flower extract, propylene carbonate, triethoxycaprylylsilane, propylparaben, aloe barbadensis leaf juice, polymethyl methacrylate, tocopheryl acetate, cocamidopropyl PG-dimonium chloride phosphate, cetyl lactate, C12-15 alkyl lactate, benzalkonium chloride, retinyl palmitate, pantothenic acid, ascorbic acid May Contain: titanium dioxide, iron oxides

### Production Example 166: Salicyclic Acid Cream

ACTIVE INGREDIENT: Salicylic Acid 0.5%
INACTIVE INGREDIENTS: Water, cyclopentasiloxane, dimethicone, kaolin, cyclomethicone, VP/hexadecene copolymer, synthetic wax, cetyl PEG/PPG-10/1 dimethicone, trisiloxane, BIS-PEG/PPG-14/14 dimethicone, cyclohexasiloxane, butylene glycol, silica, phenoxyethanol, boron nitride, polysilicone-11, quaternium-90 bentonite, sodium chloride, magnesium sulfate, propylene glycol, methylparaben, farnesol, chamomilla recutita (matricaria) flower extract, propylene carbonate, triethoxycaprylylsilane, propylparaben, aloe barbadensis leaf juice, polymethyl methacrylate, tocopheryl acetate, cocamidopropyl PG-dimonium chloride phosphate, cetyl lactate, C12-15 alkyl lactate, benzalkonium chloride, retinyl palmitate, pantothenic acid, ascorbic acid May Contain: titanium dioxide, iron oxides

### Production Example 167: Salicyclic Acid Liquid

ACTIVE INGREDIENT: Salicylic Acid 0.5%
INACTIVE INGREDIENTS: Water, cyclopentasiloxane, dimethicone, kaolin, VP/hexadecene copolymer, synthetic wax, cetyl PEG/PPG-10/1 dimethicone, trisiloxane, BIS-PEG/PPG-14/14 dimethicone, cyclohexasiloxane, butylene glycol, silica, phenoxyethanol, boron nitride, polysilicone-11, quaternium-90 bentonite, sodium chloride, magnesium sulfate, methylparaben, farnesol, chamomilla recutita (matricaria) flower extract, propylene carbonate, triethoxycaprylylsilane, propylparaben, aloe barbadensis leaf juice, polymethyl methacrylate, tocopheryl acetate, cocamidopropyl PG-dimonium chloride phosphate, cetyl lactate, C12-15 alkyl lactate, benzalkonium chloride, retinyl palmitate, pantothenic acid, ascorbic acid, titanium dioxide, iron oxides

### Production Example 168: Salicyclic Acid Cream

ACTIVE INGREDIENT: Salicylic Acid 2%
INACTIVE INGREDIENTS: Water, Cetyl Alcohol, PPG-15 Stearyl Ether, Glycolic Acid, Glycerin, Polysorbate 60, Steareth-21, C12-15 Alkyl Lactate, Cetyl Lactate, Potassium Cetyl Phosphate, Sodium Hydroxide, Xanthan Gum, Cocamidopropyl PG-Dimonium Chloride Phosphate, Disodium EDTA, Menthol, Fragrance, Benzalkonium Chloride, Panthenol

### Production Example 169: Salicyclic Acid Lotion

ACTIVE INGREDIENT: Salicylic Acid 0.5%
INACTIVE INGREDIENTS: water, dimethicone, dicaprylyl ether, glycerin, C12-15 alkyl benzoate, phenyl trimethicone, cetearyl alcohol, ceteth-10 phosphate, dicetyl phosphate, methyl gluceth-20, magnesium aluminium silicate, steareth-20, xanthan gum, steareth-2, sodium C14-16 olefin sulfonate, sodium hydroxide, cetyl lactate, benzalkonium chloride, cocamidopropyl PG-dimonium chloride phosphate, tetrasodium glutamate diacetate, C12-15 alkyl lactate, titanium dioxide, iron oxides

### Production Example 170: Salicyclic Acid Lotion

ACTIVE INGREDIENT: Salicylic Acid 0.5%
INACTIVE INGREDIENTS: water, dimethicone, dicaprylyl ether, glycerin, C12-15 alkyl benzoate, phenyl trimethicone, cetearyl alcohol, ceteth-10 phosphate, dicetyl phosphate, methyl gluceth-20, magnesium aluminium silicate, steareth-20, xanthan gum, steareth-2, sodium C14-16 olefin sulfonate, sodium hydroxide, cetyl lactate, benzalkonium chloride, cocamidopropyl PG-dimonium chloride phosphate, tetrasodium glutamate diacetate, C12-C15 alkyl lactate, titanium dioxide, iron oxides

### Production Example 171: Salicyclic Acid Lotion

ACTIVE INGREDIENT: Salicylic Acid 0.5%
INACTIVE INGREDIENTS: water, dimethicone, dicaprylyl ether, glycerin, C12-15 alkyl benzoate, phenyl trimethicone, cetearyl alcohol, ceteth-10 phosphate, dicetyl phosphate, methyl gluceth-20, magnesium aluminium silicate, steareth-20, xanthan gum, steareth-2, sodium C14-16 olefin sulfonate, sodium hydroxide, cetyl lactate, benzalkonium chloride, cocamidopropyl PG-dimonium chloride phosphate, tetrasodium glutamate diacetate, C12-15 alkyl lactate, titanium dioxide, iron oxides

### Production Example 172: Salicyclic Acid Lotion

ACTIVE INGREDIENT: Salicylic Acid 0.5%
INACTIVE INGREDIENTS: water, dimethicone, dicaprylyl ether, glycerin, C12-15 alkyl benzoate, phenyl trimethicone, cetearyl alcohol, ceteth-10 phosphate, dicetyl phosphate, methyl gluceth-20, magnesium aluminium silicate, steareth-20, xanthan gum, steareth-2, sodium C14-16 olefin sulfonate, sodium hydroxide, cetyl lactate, benzalkonium chloride, cocamidopropyl PG-dimonium chloride phosphate, tetrasodium glutamate diacetate, C12-15 alkyl lactate, titanium dioxide, iron oxides

### Production Example 173: Salicyclic Acid Lotion

ACTIVE INGREDIENT: Salicylic Acid 0.5%
INACTIVE INGREDIENTS: water, dimethicone, dicaprylyl ether, glycerin, C12-15 alkyl benzoate, phenyl trimethicone, cetearyl alcohol, ceteth-10 phosphate, dicetyl phosphate, methyl gluceth-20, magnesium aluminium silicate, steareth-20, xanthan gum, steareth-2, sodium C14-16 olefin sulfonate, sodium hydroxide, cetyl lactate, benzalkonium chloride, cocamidopropyl PG-dimonium chloride phosphate, tetrasodium glutamate diacetate, C12-15 alkyl lactate, titanium dioxide, iron oxides

### Production Example 174: Salicyclic Acid Cream

ACTIVE INGREDIENT: Salicylic Acid 0.5%
INACTIVE INGREDIENTS: Dimethicone, Zinc Stearate, Polyethylene, Octyldodecyl Stearoyl Stearate, C12-15 Alkyl Benzoate, Polysilicone-11, Nylon-12, Petrolatum, Ethylene/Acrylic Acid Copolymer, Calcium Silicate, Methylparaben, Sodium Dehydroacetate, Tocopheryl Acetate, Retinyl Palmitate, Propylparaben, Panthenol, Polymethyl Methacrylate, Ascorbyl Palmitate, C12-15 Alkyl Lactate, Cetyl Lactate, Benzalkonium Chloride, Cocamidopropyl PG-Dimonium Chloride Phosphate, Ascorbic Acid, Pantothenic Acid, Talc, Iron Oxides, Mica, Zinc Oxide.
May Contain: Titanium Dioxide, Carmine.

### Production Example 175: Salicyclic Acid Cream

ACTIVE INGREDIENT: Salicylic Acid 0.5%
INACTIVE INGREDIENTS: Dimethicone, Zinc Stearate, Polyethylene, Octyldodecyl Stearoyl Stearate, C12-15 Alkyl Benzoate, Polysilicone-11, Nylon-12, Petrolatum, Ethylene/Acrylic Acid Copolymer, Calcium Silicate, Methylparaben, Sodium Dehydroacetate, Tocopheryl Acetate, Retinyl Palmitate, Propylparaben, Panthenol, Polymethyl Methacrylate, Ascorbyl Palmitate, C12-15 Alkyl Lactate, Cetyl Lactate, Benzalkonium Chloride, Cocamidopropyl PG-Dimonium Chloride Phosphate, Ascorbic Acid, Pantothenic Acid, Talc, Iron Oxides, Mica, Zinc Oxide.
May Contain: Titanium Dioxide, Carmine.

### Production Example 176: Salicyclic Acid Gel

ACTIVE INGREDIENT: Salicylic Acid 2%
INACTIVE INGREDIENTS: Water, PPG-14 Butyl Ether, Glycerin, Cetearyl Alcohol, Cyclopentasiloxane, Stearamidopropyl Dimethylamine, Glycol Stearate, Cetyl Alcohol, Polyquaternium-37, Ceteth-2, Propylene Glycol Dicaprylate/Dicaprate, Dipalmitoylethyl Hydroxyethylmonium Methosulfate, Linoleamidopropyl PG-Dimonium Chloride Phosphate, Behentrimonium Chloride, Fragrance, Disodium EDTA, Behentrimonium Methosulfate, Dimethiconol, Ceteareth-20, PPG-1 Trideceth-6

### Production Example 177: Salicyclic Acid Lotion

ACTIVE INGREDIENT: Salicylic Acid 2%
INACTIVE INGREDIENTS: Water, Cocamidopropyl Hydroxysultaine, Sodium C14-16 Olefin Sulfonate, Sorbitol, Cellulose, Sodium Hydrolyzed Potato Starch Dodecenylsuccinate, Acrylates Crosspolymer-4, Fragrance, Sodium Hydroxide, Microcrystalline Wax, Menthol, Disodium EDTA, C12-15 Alkyl Lactate, Benzalkonium Chloride, Cocamidopropyl PG-Dimonium Chloride Phosphate

### Production Example 178: Salicyclic Acid Lotion

ACTIVE INGREDIENT: Salicylic Acid 2%
INACTIVE INGREDIENTS: Alcohol Denat., Water, Hamamelis Virginiana (Witch Hazel) Water, Polyacrylamide, Glycerin, Cyclopentasiloxane, C13-14 Isoparaffin, C12-15 Alkyl Lactate, Alcohol, Phenoxyethanol, Cetyl Lactate, PPG-2 Isoceteth-20 Acetate, Cocamidopropyl PG-Dimonium Chloride Phosphate, Laureth- 7, Polysorbate 20, Phenethyl Dimethicone, Fragrance, Dehydroxanthan Gum, Propylene Glycol, Benzalkonium Chloride, Ammonia, Tetrasodium EDTA, Butylene Glycol, Capryloyl Glycine, Sarcosine, Cinnamomum Zeylanicum Bark Extract, Crus Atlantica Bark Extract, Portulaca Oleracea Extract

### Production Example 179: Salicyclic Acid Solution

ACTIVE INGREDIENT: Salicylic Acid 0.5%
INACTIVE INGREDIENTS: Water, SD Alcohol 40-B, Glycerin, Isoceteth-20, Fragrance, PEG-32, Sodium Citrate, Benzophenone-4, Dimethicone Propyl PG-Betaine, Citric Acid, Sodium Citrate, Blue 1

### Production Example 180: Salicyclic Acid Solution

ACTIVE INGREDIENT: Salicylic Acid 2%
INACTIVE INGREDIENTS: Water, Glycerin, Cocamidopropyl Betaine, Sodium Hydrolyzed Potato Starch Dodecenylsuccinate, Decyl Glucoside, Sodium Chloride, Fragrance, Sodium Hydroxide, PEG-120 Methyl Glucose Dioleate, Menthol, Aloe Barbadensis Leaf Extract, Mentha Piperita (Peppermint) Leaf Extract

### Production Example 181: Salicyclic Acid Gel

ACTIVE INGREDIENT: Salicylic Acid 2%
INACTIVE INGREDIENTS: Water, Cocamidopropyl Hydroxysultaine, Sodium Methyl Cocoyl Taurate, Glycerin, Sodium Chloride, Sodium Hydrolyzed Potato Starch Dodecenylsuccinate, Sodium Hydroxide, Fragrance, PEG-120 Methyl Glucose Dioleate, Menthol, C12-15 Alkyl Lactate, Disodium EDTA, Cocamidopropyl PG-Dimonium Chloride Phosphate, Benzalkonium Chloride, Aloe Barbadensis Leaf Extract, Mentha Piperita (Peppermint) Leaf Extract

### Production Example 182: Salicyclic Acid Cream

ACTIVE INGREDIENT: Salicylic Acid 1%
INACTIVE INGREDIENTS: Water, Glycerin, Kaolin, Bentonite, Sodium Methyl Cocoyl Taurate, Trideceth-9, C12-15 Alkyl Lactate, PEG-5 Ethylhexanoate, Xanthan Gum, Citric Acid, Sodium Hydroxide, Cetyl Lactate, Cocamidopropyl PG-Dimonium Chloride Phosphate, Fragrance, Sodium Citrate, Disodium EDTA, Chlorphenesin, Menthol, Benzalkonium Chloride, Mentha Piperita (Peppermint) Leaf Extract, Aloe Barbadensis Leaf Extract, Titanium Dioxide

### Production Example 183: Salicyclic Acid Gel

ACTIVE INGREDIENT: Salicylic Acid 2%
INACTIVE INGREDIENTS: Water, Sodium C14-16 Olefin Sulfonate, Cocamidopropyl Hydroxysultaine, Sorbitol, Cellulose, Sodium Hydrolyzed Potato Starch Dodecenylsuccinate, Acrylates Crosspolymer-4, Sodium Hydroxide, Fragrance, Microcrystalline Wax, Menthol, C12-15 Alkyl Lactate, Benzalkonium Chloride, Disodium EDTA, Glycerin, Cocamidopropyl PG-Dimonium Chloride Phosphate, Aloe Barbadensis Leaf Extract, Mentha Piperita (Peppermint) Leaf Extract, Ferric Ferrocyanide

### Production Example 184: Salicyclic Acid Liquid

ACTIVE INGREDIENT: Salicylic Acid 2%
INACTIVE INGREDIENTS: Water, Sodium C14-16 Olefin Sulfonate, Cocamidopropyl Betaine, Glycerin, Decyl Glucoside, Acrylates Copolymer, Potassium C12-13 Alkyl Phosphate, Fragrance, Propylene Glycol, Glycol Distearate, Citric Acid, Cocamidopropyl Betaine, Laureth-4, Sodium Hydroxide, Butylene Glycol, Capryloyl Glycine, Sarcosine, Cinnamomum Zeylanicum Bark Extract, Cedrus Atlantica Bark Extract, Portulaca Oleracea Extract

### Production Example 185: Salicyclic Acid Liquid

ACTIVE INGREDIENT: Salicylic Acid 2%
INACTIVE INGREDIENTS: Water, Sodium C14-16 Olefin Sulfonate, Cocamidopropyl Betaine, PEG-120 Methyl Glucose Dioleate, Linoleamidopropyl PG-Dimonium Chloride Phosphate, Propylene Glycol, Polysorbate 20, Fragrance, PEG-80 Sorbitan Laurate, Disodium EDTA, Sodium Hydroxide, C12-15 Alkyl Lactate, Benzalkonium Chloride, Sodium Benzotriazolyl Butylphenol Sulfonate, Cocamidopropyl PG-Dimonium Chloride Phosphate, Polyquaternium-7, Citric Acid

### Production Example 186: Salicyclic Acid Lotion

ACTIVE INGREDIENT: Salicylic Acid 0.5%
INACTIVE INGREDIENTS: Water, C12-15 Alkyl Benzoate, Emulsifying Wax NF, Dimethicone, PPG-10 Cetyl Ether, C12-15 Alkyl Lactate, Methyl Gluceth-20, Cetearyl Alcohol, Glycine Soja (Soybean) Seed Extract, Phenyl Trimethicone, Steareth-21, Isoceteth-20, Polyacrylamide, Phenoxyethanol, Cetyl Lactate, Ethylene/Acrylic Acid Copolymer, Glycerin, Cocamidopropyl PG-Dimonium Chloride Phosphate, Fragrance, C13-14 Isoparaffin, Cetearyl Glucoside, Laureth-7, Pentaerythrityl Tetra-di-t-butyl Hydroxyhydrocinnamate, Benzalkonium Chloride, Disodium EDTA, Propylene Glycol, Butylene Glycol, Capryloyl Glycine, Sarcosine, Cinnamomum Zeylanicum Bark Extract, Cedrus Atlantica Bark Extract, Portulaca Oleracea Extract

### Production Example 187: Salicyclic Acid Lotion

ACTIVE INGREDIENT: Salicylic Acid 2%
INACTIVE INGREDIENTS: Alcohol Denat., Water, Hamamelis Virginiana (Witch Hazel) Water, Polyacrylamide, Glycerin, Cyclopentasiloxane, C13-14 Isoparaffin, C12-15 Alkyl Lactate, Alcohol, Phenoxyethanol, Cetyl Lactate, PPG-2 Isoceteth-20 Acetate, Cocamidopropyl PG-Dimonium Chloride Phosphate, Laureth-7, Polysorbate 20, Phenethyl Dimethicone, Fragrance, Dehydroxanthan Gum, Propylene Glycol, Benzalkonium Chloride, Ammonia, Tetrasodium EDTA, Butylene Glycol, Capryloyl Glycine, Sarcosine, Cinnamomum, Zeylanicum Bark Extract, Cedrus Atlantica Bark Extract, Portulaca Oleracea Extract

### Production Example 188: Salicyclic Acid Lotion

ACTIVE INGREDIENT: Salicylic Acid 2%
INACTIVE INGREDIENTS: Water, SD Alcohol 40-B, Hamamelis Virginiana (Witch Hazel) Water, Polyacrylamide, Glycerin, C13-14 Isoparaffin, Cyclopentasiloxane, C12-15 Alkyl Lactate, Alcohol, Laureth-7,Phenoxyethanol, Cetyl Lactate, PPG-2 Isoceteth-20 Acetate, Cocamidopropyl PG-Dimonium Chloride Phosphate, Polysorbate 20, Phenethyl Dimethicone, Fragrance, Dehydroxanthan Gum, Propylene Glycol, Benzalkonium Chloride, Ammonium Hydroxide, Tetrasodium EDTA, Butylene Glycol, Capryloyl Glycine, Sarcosine, Cinnamomum Zeylanicum Bark Extract, Cedrus Atlantica Bark Extract, Portulaca Oleracea Extract

### Production Example 189: Salicyclic Acid Lotion

ACTIVE INGREDIENT: Salicylic Acid 2%
INACTIVE INGREDIENTS: Alcohol Denat., Hamamelis Virginiana (Witch Hazel) Water, Water, Alcohol, Polyquaternium-37, Glycerin, Glycolic Acid, Butylene Glycol, Cyclopentasiloxane, C12-15 Alkyl Lactate, Isodecyl Laurate, Sodium Hydroxide, Cetyl Lactate, Bisabolol, Polysorbate 20, Cocamidopropyl PG-Dimonium Chloride Phosphate, Fragrance, Benzalkonium Chloride, Sodium Benzotriazolyl Butylphenol Sulfonate, Portulaca Oleracea Extract

### Production Example 190: Salicyclic Acid Cream

ACTIVE INGREDIENT: Salicylic Acid 0.5%
INACTIVE INGREDIENTS: Water, Cetyl Alcohol, PPG-15 Stearyl Ether, Glycerin, Steareth-21, Titanium Dioxide, Polysorbate 60, Oryza Sativa (Rice) Seed Protein, Potassium Cetyl Phosphate, Phenoxyethanol, Caprylyl Glycol, Xanthan Gum, Fragrance, Disodium Lauroamphodiacetate, Cocamidopropyl PG-Dimonium Chloride Phosphate, Disodium EDTA, C12-15 Alkyl Lactate, Menthol, Benzalkonium Chloride, Sodium Benzotriazolyl Butylphenol Sulfonate

### Production Example 191: Salicyclic Acid Paste

ACTIVE INGREDIENT: Salicylic Acid 0.5%
INACTIVE INGREDIENTS: Water, Hydrated Silica, C12-15 Alkyl Benzoate, Propylene Glycol, Glycerin, Isoceteth-20, PEG-6 Caprylic/Capric Glycerides, Titanium Dioxide, Phenoxyethanol, Cocamidopropyl Betaine, Xanthan Gum, Sodium Citrate, Dimethicone Propyl PG-Betaine, Fragrance, Menthyl Lactate, Cocamidopropyl PG-Dimonium Chloride Phosphate, C12-15 Alkyl Lactate, Citric Acid, Benzalkonium Chloride, Blue 1

### Production Example 192: Salicyclic Acid Cream

ACTIVE INGREDIENT: Salicylic Acid 0.5%
INACTIVE INGREDIENTS: Water, Hydrated Silica, C12-15 Alkyl Benzoate, Propylene Glycol, Glycerin, Isoceteth-20, PEG-6 Caprylic/Capric Glycerides, Phenoxyethanol, Cocamidopropyl Betaine, Xanthan Gum, Sodium Citrate, Fragrance, Dimethicone Propyl PG-Betaine, Menthol Lactate, Cocamidopropyl PG-Dimonium Chloride Phosphate, C12-15 Alkyl Lactate, Benzalkonium Chloride, Citric Acid, Titanium Dioxide, Blue 1

### Production Example 193: Salicyclic Acid Cream

ACTIVE INGREDIENT: Salicylic Acid 2%
INACTIVE INGREDIENTS: Water, Cetyl alcohol, PPG-15 Stearyl Ether, Cellulose, Glycerin, Polysorbate 60, Steareth-21, Microcrystalline Wax, Potassium Cetyl Phosphate, Xanthan Gum, Fragrance, Disodium EDTA, Methyl Lactate, Sodium Hydroxide, Ferric Ferrocyanide

### Production Example 194: Salicyclic Acid Strip

ACTIVE INGREDIENT: Salicylic Acid 2%
INACTIVE INGREDIENTS: Polyvinyl Alcohol, Cellulose, Sodium C14-16 Olefin Sultanate, Cocamidopropyl Hydroxysultaine, Glycerin, Water, Glycolic Acid, Menthol, Fragrance, Sodium Hydroxide, Menthyl Lactate, Disodium EDTA, Citric Acid, Red 33, Blue 1

### Production Example 195: Salicyclic Acid Kit

ACTIVE INGREDIENT: Salicylic Acid 2%
INACTIVE INGREDIENTS: Water, SD Alcohol 40-B, Isoceteth-20, Fragrance, Propylene Glycol, Dimethicone Propyl PG-Betaine, Benzophenone-4, Red 4

### Production Example 196: Salicyclic Acid Liquid

ACTIVE INGREDIENT: Salicylic Acid 2%
INACTIVE INGREDIENTS: alcohol (41%), benzophenone-4, denatonium benzoate, dimethicone propyl PG-betaine, fragrance, isoceteth-20, water, red 4

### Production Example 197: Salicyclic Acid Lotion

ACTIVE INGREDIENT: Salicylic Acid 0.5%
INACTIVE INGREDIENTS: Water, Dicaprylyl Ether, Cetearyl Alcohol, Glycerin, Neopentyl Glycol, Diethylhexanoate, Dimethicone, Methyl Gluceth-20, Menthyl Lactate, Aluminum Starch Octenylsuccinate, Ceteth-10 Phosphate, Dicetyl Phosphate, Steareth-20, Steareth-2, Neopentyl Glycol Diisostearate, Fragrance, Sodium Hydroxide, Hydrogenated Lecithin, Xanthan Gum, Magnesium Aluminum Silicate, Disodium EDTA, BHT, Titanium Dioxide

### Production Example 198: Salicyclic Acid Liquid

ACTIVE INGREDIENT: Salicylic Acid 2%
INACTIVE INGREDIENTS: Water, SD Alcohol 40-B, Isoceteth-20, Fragrance, Propylene Glycol, Dimethicone Propyl PG-Betaine, Benzophenone-4, Red 4

### Production Example 199: Salicyclic Acid Liquid

ACTIVE INGREDIENT: Salicylic Acid 0.5%
INACTIVE INGREDIENTS: Water, SD Alcohol 40-B, Glycerin, Isoceteth-20, PEG-32, Sodium Citrate, Fragrance, Propylene Glycol, Dimethicone Propyl PG-Betaine, Benzophenone-4, Algae Extract, Aloe Barbadensis Leaf Extract, Blue 1

### Production Example 200: Salicyclic Acid Lotion

ACTIVE INGREDIENT: Salicylic Acid 0.5%
INACTIVE INGREDIENTS: Water, Dicaprylyl Ether, Cetearyl Alcohol, Glycerin, Neopentyl Glycol Diethylhexanoate, Dimethicone, Phosphate, Ceteth-10 Phosphate, Methyl Gluceth-20, Menthyl Lactate, Aluminum Starch Octenylsuccinate, Steareth-20, Sodium Hydroxide, Steareth-2, Fragrance, Neopentyl Glycol Diisostearate, Lecithin, Xanthan Gum, Disodium EDTA, Magnesium Aluminum Silicate, BHT

### Production Example 201: Salicyclic Acid Liquid

ACTIVE INGREDIENT: Salicylic Acid 0.5%
INACTIVE INGREDIENTS: alcohol (24.5%), algae extract, Aloe barbadensis leaf extract, benzophenone-4, denatonium benzoate, dimethicone propyl PG-betaine, fragrance, glycerin, isoceteth-20, PEG-32, propylene glycol, sodium citrate, water, blue 1

### Production Example 202: Salicyclic Acid Cream

ACTIVE INGREDIENT: Salicylic Acid 2%
INACTIVE INGREDIENTS: water (aqua), cocamidopropyl betaine, distearyl phthalic acid amide, polyethylene, cetyl alcohol, stearyl alcohol, jojoba esters, PEG-120 methyl glucose dioleate, ammonium laureth sulfate, camellia sinensis leaf extract, aloe barbadensis leaf juice, menthol, butylene glycol, sodium hydroxide, disodium EDTA, phenoxyethanol, methylparaben, ethylparaben, butylparaben, propylparaben, isobutylparaben, fragrance (parfum), chromium hydroxide green (CI 77289)

### Production Example 203: Salicyclic Acid Gel

ACTIVE INGREDIENT: Salicylic Acid 0.5%
INACTIVE INGREDIENTS: water (aqua), alcohol denat., hamamelis virginiana (witch hazel) water, butylene glycol, alcohol, hydroxypropyl methylcellulose, citrus medica limonum (lemon) fruit extract, citrus aurantium dulcis (orange) fruit extract, camellia sinensis leaf extract, humulus lupulus (hops) extract, pyrus malus (apple) fruit extract, saccharum officinarum (sugar cane) extract, urtica dioica (nettle) extract, aloe barbadensis leaf juice, tocopheryl acetate, propylene glycol, allantoin, triethanolamine, disodium EDTA, phenoxyethanol, methylparaben, ethylparaben, propylparaben, butylparaben, isobutylparaben, green 5 (CI 61570), yellow 5 (CI 19140)

### Production Example 204: Salicyclic Acid Lotion

ACTIVE INGREDIENT: Salicylic Acid 2%
INACTIVE INGREDIENTS: water (aqua), cyclopentasiloxane, dimethicone, linoleamidopropyl PG-dimonium chloride phosphate, PPG-12/SMDI copolymer, hydroxyethyl acrylate/sodium acryloyldimethyl taurate copolymer, squalane, glyceryl stearate, PEG-100 stearate, rosmarinus officinalis (rosemary) leaf extract, melaleuca alternifolia (tea tree) extract, chamomilla recutita (matricaria) flower extract, retinyl palmitate, cetearyl alcohol, polysorbate 60, hexylene glycol, phenoxyethanol, chlorphenesin, glycerin, propylene glycol, methylparaben, benzoic acid, sodium hydroxide, fragrance parfum)

### Production Example 205: Salicyclic Acid Solution

ACTIVE INGREDIENT: Salicylic Acid 0.5%
INACTIVE INGREDIENTS: ethoxydiglycol, alcohol denat., arnica Montana flower extract, camellia oleifera leaf extract, glycyrrhiza glabra (licorice) root extract, butylenes glycol, propylene glycol, water (aqua), diazolidinyl urea, methylparaben, propylparaben

### Production Example 206: Salicyclic Acid Liquid

ACTIVE INGREDIENT: Salicylic Acid 2%
INACTIVE INGREDIENTS: alcohol denat., allantoin, ammonium hydroxide, camellia sinensis leaf extract, disodium EDTA, glycerin, glycolic acid, hamamelis virginiana (witch hazel) water, imidazolidinyl urea, panthenol, polysorbate 20, sodium benzoate, water

### Production Example 207: Salicyclic Acid Spray

ACTIVE INGREDIENT: Salicylic Acid 2%
INACTIVE INGREDIENTS: water, glycolic acid, ethyl alcohol, ammonium hydroxide, hamamelis virginiana (witch hazel) water, acetone, polysorbate 20, sodium benzoate, imidazolidinyl urea, disodium EDTA, benzoic acid

### Production Example 208: Salicyclic Acid Lotion

ACTIVE INGREDIENT: Salicylic Acid 0.5%
Inactive ingredients: water, alcohol denat., butylene glycol, cyclopentasiloxane, glyceryl stearate, PEG-100 stearate, nylon-12, PVM/MA decadiene crosspolymer, aluminum starch octenylsuccinate, cyclohexasiloxane, decylene glycol, 10-hydroxydecanoic acid, sebacic acid, 1,10-decanediol, sarcosine, xanthan gum, hexylene glycol, sodium hydroxide, disodium EDTA, caprylyl glycol, pentylene glycol, phenoxyethanol, fragrance

### Production Example 209: Salicyclic Acid Gel

ACTIVE INGREDIENT: Salicylic Acid 0.5%
INACTIVE INGREDIENTS: water, dimethicone, cyclopentasiloxane, glycereth-26, glycerin, cyclohexasiloxane, PEG-80 sorbitan laurate, dimethiconol, polyacrylate-13, panthenol, camellia sinensis leaf extract, aloe barbadensis leaf juice, butyrospermum parkii (shea) butter, bisabolol, lactic acid, caprylyl glycol, ethylhexylglycerin, PEG-40 hydrogenated castor oil, polyisobutene, hexylene glycol, polysorbate 20, PPG-26-buteth-26, butyl methoxydibenzoylmethane, ethylhexyl methoxycinnamate, ethylhexyl salicylate, phenoxyethanol, fragrance.

### Production Example 210: Salicyclic Acid Cream

ACTIVE INGREDIENT: Salicylic Acid 1%
INACTIVE INGREDIENTS: water, ethylhexyl palmitate, cetearyl alcohol, PPG-12/SMDI copolymer, cyclopentasiloxane, hydroxyethyl acrylate/sodium acryloyldimethyl taurate copolymer, squalane, hexylene glycol, chamomilla recutita (matricaria) flower extract, aleurites moluccana seed oil, retinol, sodium ascorbate, tocopherol, retinyl palmitate, ascorbic acid, panthenol, sodium hyaluronate, PEG-180, PEG-75, cetyl PEG/PPG-10/1 dimethicone, caprylic/capric triglyceride, butylene glycol, dimethicone, polysorbate 60, glyceryl stearate, PEG-100 stearate, dicetyl phosphate, ceteth-10 phosphate, sodium hydroxide, phenoxyethanol, methylparaben, ethylparaben, propylparaben, fragrance

### Production Example 211: Salicyclic Acid Cream

ACTIVE INGREDIENT: Salicylic Acid 1%
INACTIVE INGREDIENTS: water, ethylhexyl palmitate, cetearyl alcohol, PPG-12/SMDI copolymer, cyclopentasiloxane, hydroxyethyl acrylate/sodium acryloyldimethyl taurate copolymer, squalane, hexylene glycol, chamomilla recutita (matricaria) flower extract, aleurites moluccana seed oil, retinol, sodium ascorbate, tocopherol, retinyl palmitate, ascorbic acid, panthenol, sodium hyaluronate, PEG-180, PEG-75, cetyl PEG/PPG-10/1 dimethicone, caprylic/capric triglyceride, butylene glycol, dimethicone, polysorbate 60, glyceryl stearate, PEG-100 stearate, dicetyl phosphate, ceteth-10 phosphate, sodium hydroxide, phenoxyethanol, methylparaben, ethylparaben, propylparaben, fragrance

### Production Example 212: Salicyclic Acid Lotion

ACTIVE INGREDIENT: Salicylic Acid 2%
INACTIVE INGREDIENTS: water (aqua), cyclopentasiloxane, dimethicone, PPG-12/SMDI copolymer, hydroxyethyl acrylate/sodium acryloyldimethyl taurate copolymer, squalane, glyceryl stearate, PEG-100 stearate, rosmarinus officinalis (rosemary) leaf extract, melaleuca alternifolia (tea tree) extract, chamomilla recutita (matricaria) flower extract, retinyl palmitate, linoleamidopropyl PG-dimonium chloride phosphate, glycerin, cetearyl alcohol, polysorbate 60, propylene glycol, hexylene glycol, sodium hydroxide, phenoxyethanol, chlorphenesin, methylparaben, benzoic acid, fragrance (parfum)

### Production Example 213: Salicyclic Acid Liquid

ACTIVE INGREDIENT: Salicylic Acid 2%
INACTIVE INGREDIENTS: water, alcohol denat., glycolic acid, camellia sinensis leaf extract, hamamelis virginiana (witch hazel) extract, panthenol, polysorbate 20, sodium benzoate, ammonium hydroxide, disodium EDTA, allantoin, imidazolidinyl urea

### Production Example 214: Salicyclic Acid Cream

ACTIVE INGREDIENT: Salicylic Acid 1.5%
INACTIVE INGREDIENTS: caprylic/capric triglyceride, octyldodecanol, euphorbia cerifera (candelilla) wax, copernicia cerifera (carnauba) wax, jojoba esters, polyethylene, phenoxyethanol, ethylhexylglycerin, stearalkonium bentonite, dimethicone, silica, allantoin, tocopheryl acetate, ascorbyl palmitate, propylene carbonate, mica, titanium dioxide, iron oxides

### Production Example 215: Salicyclic Acid Cream

ACTIVE INGREDIENT: Salicylic Acid 1.5%
INACTIVE INGREDIENTS: caprylic/capric triglyceride, octyldodecanol, euphorbia cerifera (candelilla) wax, copernicia cerifera (carnauba) wax, jojoba esters, polyethylene, phenoxyethanol, ethylhexylglycerin, stearalkonium bentonite, dimethicone, silica, allantoin, tocopheryl acetate, ascorbyl palmitate, propylene carbonate, mica, titanium dioxide, iron oxides

### Production Example 216: Salicyclic Acid Liquid

ACTIVE INGREDIENT: Salicylic Acid 2%
INACTIVE INGREDIENTS: water, alcohol denat, hamamelis virginiana (witch hazel) extract, glycolic acid, ammonium hydroxide, polysorbate 20, sodium benzoate, imidazolidinyl urea, disodium EDTA

### Production Example 217: Salicyclic Acid Cream

ACTIVE INGREDIENT: Salicylic Acid 2%
INACTIVE INGREDIENTS: Water, Cocamidopropyl Betaine, Distearyl Phthalic Acid Amide, Polyethylene, Cetyl Alcohol, Stearyl Alcohol, PEG-120 Methyl Glucose Dioleate, Ammonium Laureth Sulfate, Sodium Chloride, Aloe Barbadensis Leaf Juice, Camellia Sinensis Leaf Extract, Microcrystalline Wax, Butylene Glycol, Disodium EDTA, Sodium Hydroxide, Menthol, Phenoxyethanol, Methylparaben, Ethylparaben, Butylparaben, Propylparaben, Isobutylparaben, Fragrance, Ferric Ferrocyanide, Blue 1

### Production Example 218: Salicyclic Acid Cream

ACTIVE INGREDIENT: Salicylic Acid 2%
INACTIVE INCREMENTS: Benzoic Acid, Butylene Cetearyl Alcohol, Cetyl Alcohol, Chlorphenesin, Disodium EDTA, Fragrance, Glycerin, Glyceryl Stearate, Glycolic Acid, Isododecane, Linoleic Acid, Linolenic Acid, Morus Alba Root Extract, Neopentyl Glycol Diheptanoate, Paraffin, PEG-100 Stearate, Phenoxyethanol, Polysorbate 20, Saxifraga Sarmentosa Extract, Scutellaria Baicalensis Root Extract, Sodium Hyaluronate, Sodium Hydroxide, Sorbic Acid, Vitis Vinifera (Grape) Fruit Extract, Water, Xanthan Gum

### Production Example 219: Salicyclic Acid Lotion

ACTIVE INGREDIENT: Salicylic Acid 2%
INACTIVE INGREDIENTS: Water, Cyclopentasiloxane, Dimethicone, PPG-12/SMDI Copolymer, Hydroxyethyl Acrylate/sodium Acryloyldimethyl Taurate Copolymer, Squalane, Glyceryl Stearate, PEG-100 Stearate, Hexylene Glycol, Cetearyl Alcohol, Linoleamidopropyl PG-Dimonium Chloride Phosphate, Rosmarinus Officinalis (Rosemary) Leaf Extract, Chamomilla Recutita (Matricaria) Flower Extract, Melaleuca Alternifolia (Tea Tree) Flower Leaf/Stem Extract, Retinyl Palmitate, Glycerin, Polysorbate 60, Propylene Glycol, Sodium Hydroxide, Phenoxyethanol, Chlorphenesin, Methylparaben, Benzoic Acid, Fragrance

### Production Example 220: Salicyclic Acid Lotion

ACTIVE INGREDIENT: Salicylic Acid 1%
INACTIVE INCREMENTS: Water, Ethylhexyl Palmitate, Cetearyl Alcohol, Cyclopentasiloxane, PPG-12/SMDI Copolymer, Hydroxyethyl Acrylate/Sodium Acryloyldimethyl Taurate Copolymer, Squalane, Hexylene Glycol, Dicetyl Phosphate, Ceteth-10 Phosphate, Chamomilla Recutita (Matricaria) Flower Extract, Aleurites Moluccana Seed Oil, Retinol, Sodium Ascorbate, Tocopherol, Retinyl Palmitate, Ascorbic Acid, PEG-180, PEG-75, PEG-8 Cetyl Dimethicone, Panthenol, Sodium Hyaluronate, Caprylic/Capric Triglyceride, Propylene Glycol, Dimethicone, Polysorbate 60, Glyceryl Stearate, PEG-100 Stearate, Sodium Hydroxide, Phenoxyethanol, Methylparaben, Ethylparaben, Butylparaben, Propylparaben, Isobutylparaben, fragrance

### Production Example 221: Salicyclic Acid Cream

ACTIVE INGREDIENT: Salicylic Acid 1%
INACTIVE INGREDIENTS: water, ethylhexyl palmitate, cetearyl alcohol, PPG-12/SMDI copolymer, cyclopentasiloxane, hydroxyethyl acrylate/sodium acryloyldimethyl taurate copolymer, squalane, chamomilla recutita (matricaria) flower extract, aleurites moluccana seed oil, retinol, sodium ascorbate, tocopherol, retinyl palmitate, ascorbic acid, panthenol, sodium hyaluronate, PEG-180, PEG-75, cetyl PEG/PPG-10/1 dimethicone, hexylene glycol, caprylic/capric triglyceride, propylene glycol, dimethicone, polysorbate 60, glyceryl stearate, PEG-100 stearate, dicetyl phosphate, ceteth-10 phosphate, sodium hydroxide, phenoxyethanol, methylparaben, ethylparaben, propylparaben, isobutylparaben, fragrance.

### Production Example 222: Salicyclic Acid Lotion

ACTIVE INGREDIENT: Salicylic Acid 0.5%
INACTIVE INGREDIENTS: water, butylene glycol, cetearyl alcohol, dimethicone, sodium polyacrylate, scutellaria baicalensis root extract, sophora angustifolia root extract, morus alba root extract, glycyrrhiza glabra (licorice) root extract, arctostaphylos uva ursi leaf extract, kojic acid, zinc gluconate, alpha-glucan oligosaccharide, sodium hyaluronate, bisabolol, allantoin, squalane, safflower oil/palm oil aminopropanediol esters, C12-15 alkyl lactate, caprylic/capric triglyceride, caprylyl glycol, ethylhexylglycerin, glycerin, ethylhexyl palmitate, hydrogenated polydecene, hydroxypropyl cyclodextrin, PPG-5 laureth-5, polyquaternium-11, ceteareth-20, aluminum starch octenylsuccinate, polysorbate 60, hexylene glycol, PPG-2 myristyl ether propionate, sodium hydroxide, disodium EDTA, phenoxyethanol, fragrance

### Production Example 223: Salicyclic Acid Cream

ACTIVE INGREDIENT: Salicylic Acid 2%
INACTIVE INGREDIENTS: water, butylene glycol, hamamelis virginiana (witch hazel) water, glycolic acid, sodium hydroxide, portulaca oleracea extract, ethylhexylglycerin, polysorbate 20, disodium EDTA, sodium benzoate, phenoxyethanol

### Production Example 224: Salicyclic Acid Cream

ACTIVE INGREDIENT: Salicylic Acid 1.5%
INACTIVE INGREDIENTS: caprylic/capric triglyceride, octyldodecanol, euphorbia cerifera (candelilla) wax, copernicia cerifera (carnauba) wax, jojoba esters, polyethylene, phenoxyethanol, ethylhexylglycerin, stearalkonium bentonite, dimethicone, silica, allantoin, tocopheryl acetate, ascorbyl palmitate, propylene carbonate, mica, titanium dioxide, iron oxides

### Production Example 225: Salicyclic Acid Liquid

ACTIVE INGREDIENT: Salicylic Acid 2%
INACTIVE INGREDIENTS: water (aqua), butylene glycol, hamamelis virginiana (witch hazel) water, glycolic acid, sodium hydroxide, portulaca oleracea extract, polysorbate 20, ethylhexylglycerin, disodium EDTA, phenoxyethanol, sodium benzoate, fragrance (parfum)

### Production Example 226: Salicyclic Acid Gel

ACTIVE INGREDIENT: Salicylic Acid 2%
INACTIVE INGREDIENTS: Water, Butylene Glycol, Niacinamide, Cyclopentasiloxane, Dimethicone, Hydroxyethyl Acrylate/Sodium Acryloyldimethyl Taurate Copolymer, Acrylates/C10-30 Alkyl Acrylate Crosspolymer, Cetyl Ethylhexanoate, Dimethicone/Vinyl Dimethicone Crosspolymer, Silica, Propanediol, Polygonum Cuspidatum Root Extract, Myristyl Alcohol, Tetrasodium EDTA, PCA, Ethylhexylglycerin, Sodium Hyaluronate, Sodium Hydroxide, 1,2-Hexanediol, Caprylyl Glycol, Isohexadecane, Polysorbate 60, Phenoxyethanol, Fragrance, Blue 1

### Production Example 227: Salicyclic Acid Lotion

ACTIVE INGREDIENT: Salicylic Acid 0.5%
INACTIVE INGREDIENTS: water (aqua), alcohol denat., butylene glycol, cyclopentasiloxane, glyceryl stearate, PEG-100 stearate, nylon-12, PVM/MA decadiene crosspolymer, aluminum starch octenylsuccinate, cyclohexasiloxane, decylene glycol, 10-hydroxydecanoic acid, sebacic acid, 1,10-decanediol, sarcosine, xanthan gum, hexylene glycol, sodium hydroxide, disodium EDTA, caprylyl glycol, pentylene glycol, phenoxyethanol, fragrance (parfum).

### Production Example 228: Salicyclic Acid Gel

ACTIVE INGREDIENT: Salicylic Acid 0.5%
INACTIVE INGREDIENTS: water, dimethicone, cyclopentasiloxane, glycereth-26, glycerin, cyclohexasiloxane, PEG-80 sorbitan laurate, dimethiconol, polyacrylate-13, panthenol, camellia sinensis leaf extract, aloe barbadensis leaf juice, butyrospermum parkii (shea) butter, bisabolol, lactic acid, caprylyl glycol, ethylhexylglycerin, PEG-40 hydrogenated castor oil, polyisobutene, hexylene glycol, polysorbate 20, PPG-26-buteth-26, butyl methoxydibenzoylmethane, ethylhexyl methoxycinnamate, ethylhexyl salicylate, phenoxyethanol, fragrance.

### Production Example 229: Salicyclic Acid Liquid

ACTIVE INGREDIENT: Salicylic Acid 2%
INACTIVE INGREDIENTS: Water (aqua), SD alcohol 40-B, hamamelis virginiana (witch hazel) extract, glycolic acid, ammonium hydroxide, polysorbate 20, sodium benzoate, imidazolidinyl urea, disodium EDTA.

### Production Example 230: Salicyclic Acid Gel

ACTIVE INGREDIENT: Salicylic Acid 0.5%
INACTIVE INGREDIENTS: water (aqua), dimethicone, cyclopentasiloxane, glycereth-26, glycerin, cyclohexasiloxane, PEG-80 sorbitan laurate, dimethiconol, polyacrylate-13, panthenol, camellia sinensis leaf extract, aloe barbadensis leaf juice, butyrospermum parkii (shea) butter, bisabolol, lactic acid, caprylyl glycol, ethylhexylglycerin, PEG-40 hydrogenated castor oil, polyisobutene, hexylene glycol, polysorbate 20, PPG-26-buteth-26, butyl methoxydibenzoylmethane, ethylhexyl methoxycinnamate, ethylhexyl salicylate, phenoxyethanol, fragrance (parfum).

### Production Example 231: Salicyclic Acid Cream

ACTIVE INGREDIENT: Salicylic Acid 0.5%
INACTIVE INGREDIENTS: Water, Alcohol Denat., Cyclopentasiloxane, PVM/MA Decadiene Crosspolymer, Dimethicone, Oligopeptide-10, sh-Oligopeptide-1, Phenoxyethanol, PPG-26-Buteth-26, Sodium Hydroxide, Butylene Glycol, Panthenol, PEG-40 Hydrogenated Castor Oil, Polysilicone-11, Saccharomyces Lysate Extract, Glycerin, Caprylyl Glycol, Chlorphenesin, Decyl Glucoside, Avena Sativa (Oat) Kernel Extract, Hexylene Glycol, Potassium Sorbate, Citrus Aurantium Dulcis (Orange) Peel Oil, Citric Acid, Pantolactone, Limonene.

### Production Example 232: Salicyclic Acid Liquid

ACTIVE INGREDIENT: Salicylic Acid 2%
INACTIVE INGREDIENTS: water, sodium laureth sulfate, decyl glucoside, glycerin, coco-betaine, mentha piperita (peppermint) leaf extract, PEG-150 pentaerythrityl tetrastearate, zinc gluconate, sodium chloride, sodium hydroxide, capryloyl salicylic acid, tetrasodium EDTA, citric acid, pentasodium ethylenediamine tetramethylene phosphonate, menthol, polyquatermium-47, fragrance

### Production Example 233: Salicyclic Acid Cream

ACTIVE INGREDIENT: Salicylic Acid 2%
INACTIVE INGREDIENTS: Water, Cetyl Alcohol, PPG-15 Stearyl Ether, Polysorbate 60, Steareth-21, Potassium Cetyl Phosphate, Xanthan Gum, Fragrance, Menthyl Lactate, Glycine Soja (Soybean) Seed Extract

### Production Example 234: Salicyclic Acid Cloth

ACTIVE INGREDIENT: Salicylic Acid 0.6%
INACTIVE INGREDIENTS: Water, Glycerin, Sodium C14-16 Olefin Sulfonate, Cocamidopropyl Betaine, PEG-16 Soy Sterol, Polysorbate 20, Sodium Benzoate, Phenoxyethanol, Fragrance, Disodium EDTA, Butylene Glycol, Sodium Hydroxide, Glycine Soja (Soybean) Protein

### Production Example 235: Salicyclic Acid Lotion

ACTIVE INGREDIENT: Salicylic Acid 0.5%
INACTIVE INGREDIENTS: Water, C12-15 Alkyl Benzoate, Cetearyl Alcohol, Dimethicone, Methyl Gluceth-20, PPG-10 Cetyl Ether, Bis-Phenylpropyl Dimethicone, Arachidyl Alcohol, Behenyl Alcohol, Glycine Soja (Soybean) Seed Extract, Glycerin, Isoceteth-20, Polyacrylamide, Cetearyl Glucoside, Arachidyl Glucoside, C13-14 Isoparaffin, Ethylene/Acrylic Acid Copolymer, Fragrance, Laureth-7, Disodium EDTA, BHT, Sodium Hydroxide, Citric Acid

### Production Example 236: Salicyclic Acid Liquid

ACTIVE INGREDIENT: Salicylic Acid 0.5%
INACTIVE INGREDIENTS: water, cocamidopropyl betaine, Sodium C14-16 olefin sulfonate, sodium lauroamphoacetate, sorbitol, polysorbate 20, glycerin, disodium lauroamphoacetate, PEG-6 caprylic/capric glycerides, sodium benzoate, fragrance, xanthan gum, citric acid, disodium EDTA, PEG-16 soy sterol, butylene glycol, glycine soja (soybean) protein

### Production Example 237: Salicyclic Acid Cream

ACTIVE INGREDIENT: Salicylic Acid 2%
INACTIVE INGREDIENTS: Water, Cetyl Alcohol, PPG-15 Stearyl Ether, Polysorbate 60, Steareth-21, Potassium Cetyl Phosphate, Xanthan Gum, Fragrance, Menthyl Lactate, Glycine Soja (Soybean) Seed Extract

### Production Example 238: Salicyclic Acid Cloth

ACTIVE INGREDIENT: Salicylic Acid 0.6%
INACTIVE INGREDIENTS: Water, Glycerin, Sodium C14-16 Olefin Sulfonate, Cocamidopropyl Betaine, PEG-16 Soy Sterol, Polysorbate 20, Sodium Benzoate, Phenoxyethanol, Fragrance, Disodium EDTA, Butylene Glycol, Sodium Hydroxide, Glycine Soja (Soybean) Protein

### Production Example 239: Salicyclic Acid Lotion

ACTIVE INGREDIENT: Salicylic Acid 0.5%
INACTIVE INGREDIENTS: Water, C12-15 Alkyl Benzoate, Cetearyl Alcohol, Dimethicone, Methyl Gluceth-20, PPG-10 Cetyl Ether, Bis-Phenylpropyl Dimethicone, Arachidyl Alcohol, Behenyl Alcohol, Glycine Soja (Soybean) Seed Extract, Glycerin, Isoceteth-20, Polyacrylamide, Cetearyl Glucoside, Arachidyl Glucoside, C13-14 Isoparaffin, Ethylene/Acrylic Acid Copolymer, Fragrance, Laureth-7, Disodium EDTA, BHT, Sodium Hydroxide, Citric Acid

### Production Example 240: Salicyclic Acid Liquid

ACTIVE INGREDIENT: Salicylic Acid 0.5%
INACTIVE INGREDIENTS: Water, Cocamidopropyl Betaine, Sodium C14-16 Olefin Sulfonate, Glycerin, Sorbitol, Disodium Lauroamphodiacetate, Polysorbate 20, PEG-6 Caprylic/Capric Glycerides, Sodium Benzoate, Fragrance, Citric Acid, Xanthan Gum, Disodium EDTA, PEG-16 Soy Sterol, Butylene Glycol, Glycine Soja (Soybean) Protein

### Production Example 241: Salicyclic Acid Kit

ACTIVE INGREDIENT: Salicylic Acid 0.5%
INACTIVE INGREDIENTS: Water, Cocamidopropyl Betaine, Sodium C14-16 Olefin Sulfonate, Sodium Lauroamphoacetate, Sorbitol, Polysorbate 20, Glycerin, Disodium Lauroamphodiacetate, PEG-6 Caprylic/Capric Glycerides, Sodium Benzoate, Fragrance, Xanthan Gum, Citric Acid, Disodium EDTA, PEG-16 Soy Sterol, Butylene Glycol, Glycine Soja (Soybean) Protein

### Production Example 242: Salicyclic Acid Swab

ACTIVE INGREDIENT: Salicylic Acid 0.5%
INACTIVE INGREDIENTS: Aloe barbadensis leaf juice, bilberry (Vaccinium myrtillus) fruit extract, caprylyl glycol, citric acid, disodium EDTA, disodium tetramethylhexadecenylcysteine formylprolinate, lauryl glucoside, lemon (Citrus limon) fruit extract, niacinamide, orange (Citrus aurantium dulcis) fruit extract, phenoxyethanol, PPG-5 ceteth-20, sodium hydroxide, sodium lauryl glucose carboxylate, sugar cane (Saccharum officinarum) extract, sugar maple (Acer saccharum) extract, water, witch hazel (Hamamelis virginiana) bark/leaf/twig extract.

### Production Example 243: Salicyclic Acid Cream

ACTIVE INGREDIENT: Salicylic Acid 2%
INACTIVE INGREDIENTS: Aloe Barbadensis Leaf Juice, Nerium Oleander Leaf Extract (NAE8®), Water (Aqua), Propanediol, Hydrolyzed Egg Shell Membrane (Peptide Matrix—Collagen & Elastin), Cetyl Alcohol, Glycerin, Glyceryl Stearate, Potassium Cetyl Phosphate, Cetearyl Olivate, Caprylic/Capric Triglyceride, Dimethicone, Ricinus Communis (Castor) Seed Oil, Glyceryl Caprylate, Sorbitan Olivate, Lysolecithin, Sclerotium Gum, Xanthan Gum, Allantoin, Arginine, Avena Sativa Kernel Flour (Colloidal Oatmeal), Butyrospermum Parkii (Shea) Butter, Olea Europaea (Olive) Fruit Oil, Pelargonium Graveolens (Geranium) Flower Oil, Tocopherol, Fragrance, Leuconostoc/Radish Root Ferment Filtrate, Pullulan, Ocimum Basilicum (Basil) Extract, Linum Usitatissimum (Linseed) Seed Oil, Carum Petroselinum (Parsley) Extract, Benzyl Alcohol, Potassium Sorbate, Dipotassium Glycyrrhizate, Glyceryl Undecylenate, Sea Salt, Mahonia Aquifolium (Oregon Grape) Root Extract, Sodium Benzoate, Muriella Aurantiaca/Ruttnera Lamellosa Oil (Plankton Extract), Citric Acid, Lecithin

### Production Example 244: Salicyclic Acid Lotion

ACTIVE INGREDIENT: Salicylic Acid 1%
INACTIVE INGREDIENTS: water\aqua\eau, dimethicone, butylene glycol, vinyl dimethicone/methicone silsesquioxane crosspolytner, methyl trimethicone, polysorbate 20, bis-peg-18 methyl ether dimethyl silane, lauryl peg-9 polydimethylsiloxyethyl dimethicone, glycerin, methyl gluceth-20, polysilicone-11, oenothera biennis (evening primrose) flower extract, glycine soja (soybean) seed extract, sigesbeckia orientalis (st. paul's wort) extract, salvia sclarea (clary) extract, plankton extract•laminaria saccharina extract, acetyl hexapeptide-8, acetyl glucosamine, caffeine, gylcine soja (soybean) protein, aminopropyl ascorbyl phosphate, cholesterol, caprylyl glycol, micrococcus lysate, palmitoyl hexapeptide-12, lecithin, whey protein\lactis protein\protéine du petit-lait, sodium hyaluronate, tocopheryl acetate, peg-8, sodium hydroxide, ammonium acryloyldimethyltaurate/vp copolymer, xanthan gum, carbomer, glyceryl polymethacrylate, sodium benzoate, sodium citrate, disodium edta, phenoxyethanol [iln42038]

### Production Example 245: Salicyclic Acid Gel

ACTIVE INGREDIENT: Salicylic Acid 2%
INACTIVE INGREDIENTS: water\aqua\eau, glycerin, sodium laureth sulfate, sodium chloride, lauramidopropyl betaine•butylene glycol, sucrose, gentiana lutea (gentian) root extract, laminaria saccharina extract, caffeine, sodium hyaluronate, acetyl glucosamine, laureth-2, peg-120 methyl glucose dioleate, sodium sulfate, benzophenone-4, sodium hydroxide, edta, disodium edta, bht, phenoxyethanol, blue 1 (ci 42090) [iln41073]

### Production Example 246: Salicyclic Acid Liquid

ACTIVE INGREDIENT: Salicylic Acid 2%
INACTIVE INGREDIENTS: water, glycerin, butylene glycol, sodium methyl cocoyl taurate, sucrose, disodium phosphate, arginine cocoate, laminaria saccharina extract, cola nitida (kola) seed extract, caffeine, algae extract, mentha piperita (peppermint), sea whip extract, peg/ppg-18/18 dimethicone, sodium hyaluronate, ppg-6 decyltetradeceth-30, lactobacillus ferment, stearamidopropyl dimethylamine, longifolene, acetyl glucosamine, capryloyl glycine, perilla aldehyde, 10-hydroxydecanoic acid, polyquaternium-7, beta-caryophyllene, phospholipids, sodium stearate, sodium salicylate, disodium edta, phenoxyethanol, chloroxylenol

### Production Example 247: Salicyclic Acid Liquid

ACTIVE INGREDIENT: Salicylic Acid 1.5%
INACTIVE INGREDIENTS: water, alcohol denat., nylon-12, butylene glycol, Hamamelis virginiana (witch hazel), laminaria saccharina extract, caffeine, sucrose, glycerin, acetyl glucosamine, sorbitol, sea whip extract, barium sulfate, 10-hydroxydecanoic acid, silica, disodium edta, benzalkonium chloride

### Production Example 248: Salicyclic Acid Lotion

ACTIVE INGREDIENT: Salicylic Acid 1%
INACTIVE INGREDIENTS: water, butylene glycol, cetyl esters, stearic acid, kaolin, glyceryl dilaurate, hydrogenated polyisobutene, steareth-21, laminaria saccharina extract, caffeine, peg-150 pentaerythrityl tetrastearate, steareth-2, glyceryl stearate, lecithin, acetyl glucosamine, cellulose gum, 10-hydroxydecanoic acid, magnesium aluminum silicate, silica, xanthan gum, aminomethyl propanediol, simethicone, tocopheryl acetate, disodium edta, phenoxyethanol, potassium sorbate may contain: mica, titanium dioxide, iron oxides, chromium oxide greens iln35126

### Production Example 249: Salicyclic Acid Liquid

ACTIVE INGREDIENT: Salicylic Acid 0.5%
INACTIVE INGREDIENT: methyl trimethicone, water, dipropylene glycol, alcohol denat., nylon-12, butylene glycol, talc, peg-10 dimethicone, dimethicone, trimethylsiloxysilicate, silica, laminaria saccharina extract, calcium stearate, isononyl isononanoate, caffeine, glycerin, hydroxyapatite, dimethicone/peg-10/15 crosspolymer, sorbitan sesquioleate, dimethicone/vinyl dimethicone crosspolymer, triethoxycaprylylsilane, quaternium-90 bentonite, 10-hydroxydecanoic acid, lecithin, tocopherol, sodium chloride, tromethamine, propylene carbonate, alumina, disodium edta, sodium citrate, phenoxyethanol may contain: titanium dioxide, iron oxides, mica iln36140

### Production Example 250: Salicyclic Acid Cream

ACTIVE INGREDIENT: Salicylic Acid 1%
INACTIVE INGREDIENTS: water, kaolin, butylene glycol, magnesium aluminum silicate, sodium lauroyl sarcosinate, glyceryl stearate, peg-100 stearate, gentiana lutea (gentian) root extract, laminaria saccharina extract, sucrose, chamomilla recutita (matricaria), algae extract, aloe barbadensis powder, acetyl glucosamine, inulin, 2-hexanediol, caprylyl glycol, xanthan gum, disodium edta, chlorphenesin, phenoxyethanol, titanium dioxide iln34859

### Production Example 251: Salicyclic Acid Gel

ACTIVE INGREDIENT: Salicylic Acid 0.5%
INACTIVE INGREDIENTS: alcohol denat., water, acrylates/c10-30 alkyl acrylate crosspolymer, Hamamelis virginiana (witch hazel), laminaria saccharina extract, sea whip extract, butylene glycol, benzalkonium chloride

### Production Example 252: Salicyclic Acid Gel

ACTIVE INGREDIENT: Salicylic Acid 2%
INACTIVE INGREDIENTS: water, sodium laureth sulfate, perlite, sodium chloride, acrylates copolymer, sodium hydroxide, coco-betaine, cocamide MEA, glycerin, citric acid, trisodium ethylenediamine disuccinate, hydrogenated castor oil, propylene glycol, hydrogenated jojoba oil, iron oxides, charcoal powder, sorbitol, Vaccinium myrtillus fruit extract, polyglycerin-10, polyglyceryl-10 myristate, polyglyceryl-10 stearate, sodium dehydroacetate, PPG-5-ceteth-20

### Production Example 253: Salicyclic Acid Spray

ACTIVE INGREDIENT: Salicylic Acid 2%
INACTIVE INGREDIENTS: Water, Alcohol Denat., Hamamelis virginiana (Witch Hazel) Extract, Sorbitol, Glycerin, Glycolic Acid, Polysorbate 20, Sodium Hydroxide, Benzalkonium Chloride, Bisabolol, Zingiber Officinale (Ginger) Root Extract, Cocamidopropyl Dimethylamine, Polysorbate 80, Phenoxyethanol, Ethylhexylglycerin, Carthamus Tinctorius (Safflower) Seed Oil, Clea Europaea (Olive) Fruit Oil, Chamomila Recutita (Matricaria) Flower Extract, Leptogorgia Virgulata (Sea Whip) Extract, Butylene Glycol, Aloe Barbadensis Leaf Juice, Panthenol. Methylisothiazolinone, Niacinamide, Disodium EDTA, Lactic Acid

### Production Example 254: Salicyclic Acid Gel

ACTIVE INGREDIENT: Salicylic Acid 2%
INACTIVE INGREDIENTS: Alcohol, Allium Fistulosum Bulb Extract, Backhousia Citriodora Leaf Extract, Butylene Glycol, Butylparaben, Cardiospermum Halicacabum Flower/Leaf/Vine Extract, Citric Acid, Echium Plantagineum Seed Oil, Ethylparaben, Hamamelis virginiana (Witch hazel) Water, Helianthus Annuus (Sunflower) Seed Oil Unsaponifiables, Hydroxypropylcellulose, Isobutylparaben, Methylparaben, Octyldodecanol, Phenoxyethanol, Potassium Benzoate, Potassium Sorbate, Propylparaben, Santalum Album (Sandalwood) Oil, SD Alcohol 40-B, Tocopherol, Water

### Production Example 255: Salicyclic Acid Gel

ACTIVE INGREDIENT: Salicylic Acid 0.5%
INACTIVE INGREDIENTS: Anhydrous Citric Acid, Alcohol Denatured, Benzyl Alcohol, Glycerin, Glyceryl Caprylate, Hydrolyzed Algin, Illite, Kaolin, Lonicera Caprifolium (Honeysuckle) Flower Extract, Lonicera Japonica (Honeysuckle) Flower Extract, Montmorillonite, Polysorbate 80, Santalum Album (Sandalwood) Oil, Santalum Spicatum (Sandalwood) Powder, Terminalia Ferdinandiana Fruit Extract, Water, Zinc Sulfate

### Production Example 256: Salicyclic Acid Cream

ACTIVE INGREDIENT: Salicylic Acid 1%
INACTIVE INGREDIENTS; water, kaolin, glycerin, butylene glycol, magnesium aluminum silicate, glyceryl stearate, peg-100 stearate, silica, sodium lauroyl sarcosinate, allyl methacrylates crosspolymer, gentiana lutea (gentian) root extract, laminaria saccharina extract, sucrose, acetyl glucosamine, caprylyl glycol, 1,2-hexanediol, tocopheryl acetate, sodium hydroxide, xanthan gum, disodium edta, phenoxyethanol, titanium dioxide, blue 1, yellow 5.

### Production Example 257: Salicyclic Acid Cream

ACTIVE INGREDIENT: Salicylic Acid 1%
INACTIVE INGREDIENTS; water, kaolin, glycerin, butylene glycol, magnesium aluminum silicate, glyceryl stearate, peg-100 stearate, silica, sodium lauroyl sarcosinate, allyl methacrylates crosspolymer, gentiana lutea (gentian) root extract, laminaria saccharina extract, sucrose, acetyl glucosamine, caprylyl glycol, 1,2-hexanediol, tocopheryl acetate, sodium hydroxide, xanthan gum, disodium edta, phenoxyethanol, titanium dioxide, blue 1, yellow 5.

### Production Example 258: Salicyclic Acid Cream

ACTIVE INGREDIENT: Salicylic Acid 1%
INACTIVE INGREDIENTS: magnesium sulfate, dimethicone, isononyl isononanoate, isododecane, polysilicone-11, phenyl trimethicone, hydrogenated lecithin, acetyl glucosamine, polysorbate 20, polysorbate 80, polyethylene, diatomaceous earth\solum diatomeae\terre de diatomées, triethylhexanoin, pumice, bambusa arundinacea (bamboo) stem extract, peg-15/lauryl dimethicone crosspolymer, caprylyl glycol, stearyl glycyrrhetinate, water\aqua\eau, silica [iln43644]

### Production Example 259: Salicyclic Acid Lotion

ACTIVE INGREDIENT: Salicylic Acid 1.5%
INACTIVE INGREDIENTS: water, coco-glucoside, propylene glycol, ammonium polyacryloyldimethyl taurate, glycerin, coco-betaine, sodium cocoyl isethionate, sodium methyl cocoyl taurate, dipropylene glycol, sodium chloride, butylene glycol, phenoxyethanol, hydrogenated coconut acid, capryloyl salicylic acid, coconut acid, t-butyl alcohol, sodium PCA, camphor, menthol, boswellia serrata extract, sodium isethionate, Hamamelis virginiana (witch hazel) water, peumus boldus leaf extract, disodium EDTA, alcohol, zingiber officinale (ginger) root extract, sanguisorba officinalis root extract, cinnamomum cassia bark extract, xanthan gum, laminaria saccharina extract, zinc sulfate, pyridoxine HCL, sodium hydroxide, citric acid

### Production Example 260: Salicyclic Acid Lotion

ACTIVE INGREDIENT: Salicylic Acid 1.5%
INACTIVE INGREDIENTS: water, coca-glucoside, propylene glycol, sorbitol, sodium laureth sulfate, treithanolamine, sodium chloride, PEG-120 methyl glucose dioleate, menthol, methylparaben, imidazolidinyl urea, benzophenone-4, disodium EDTA, dipropylene glycol, camphor, zinc PCA, aloe barbadensis leaf juice, boswella serrata extract, laminaria saccharina extract, blue 1, poterium officinale root extract, zinber officinale (ginger) root extract, cinnamomum cassia bark extract, Hamamelis virginiana (witch hazel) leaf extract

### Production Example 261: Salicyclic Acid Lotion

ACTIVE INGREDIENT: Salicylic Acid 0.5%
INACTIVE INGREDIENTS: water, glycerin, montmorillonie, dimethicone, cyclohexasiloxane, glyceryl stearate SE, polyglyceryl-3 methylglycose distearate, stearyl alcohol, pentylene glycol, propylene glycol, methylparaben, chlorphenesin, xanthan gum, carbomer, sodium hydroxide, sodium PCA, butylparaben, dipropylene glycol, tetrasodium EDTA, laminaria saccharina extract, menthol, aloe barbadensis leaf juice, bosewella serrata extract, camphor, Hamamelis virginiana (witch hazel) leaf extract, poterium officinale root extract, zinglber officinale (ginger) root extrct, cinnamomum cassia bark extract, blue 1

### Production Example 262: Salicyclic Acid Lotion

ACTIVE INGREDIENT: Salicylic Acid 1.5%
INACTIVE INGREDIENTS: water, alcohol denat., glycerin, glycolic acid, triethanolamine, ammonium polyacryloyldimethyl taurate, acrylates/C10-30 alkyl acrylate crosspolymer, pentylene glycol, butylene glycol, zinc gluconate, propylelne glycol, dipropylene glycol, camphor, menthol, aloe barbadensis leaf juice, boswellia serrata extract, laminaria saccharina extract, Hamamelis virginiana (witch hazel) leaf extract, potarium officinale root exract, zingiber officinale (ginger) root extract, cimmamoum casaia bark extract

### Production Example 263: Salicyclic Acid Lotion

ACTIVE INGREDIENT: Salicylic Acid 1.5%
INACTIVE INGREDIENTS: water, DI-C12-13 alkyl malate, cyclohexasiloxane, propylene glycol, aluminum starch octenylsuccinate, PEG-100 stearate, glyceryl stearate, cetyl alcohol, sodium hydroxide, acrylates/C10-30 alkyl acrylate crosspolymer, capryloyl salicylic acid, xanthan gum, zinc PCA, iodopropynyl butylcarbamate, octadecenedioic acid

### Production Example 264: Salicyclic Acid Lotion

ACTIVE INGREDIENT: Salicylic Acid 1.5%
INACTIVE INGREDIENTS: water, aloe barbadensis leaf juice, di-C12-13 alkyl malate, niacinamide, cyclohexasiloxane, propylene glycol, aluminum starch octenylsuccinate, PEG-100 stearate, glyceryl stearate, cetyl alcohol, acrylates/C10-30 alkyl acrylate crosspolymer, sodium hydroxide, capryloyl salicylic acid, xanthan gum, zinc PCA, zingiber officinale (ginger) root extract, stearyl alcohol, myristyl alcohol, citric acid, potassium sorbate, octadecenedioic acid, sodium benzoate, citral, BHT

### Production Example 265: Sulfur Lotion

ACTIVE INGREDIENT: Sulfur 10%
INACTIVE INGREDIENTS: water, niacinamide, zea mays (corn) starch, glycolic acid, stearyl alcohol, glycerin, octyldodecyl myristate, cetearyl alcohol, glyceryl stearate, magnesium aluminum silicate, phenoxyethanol, polyacrylamide, ceteareth-20, sodium hydroxide, PEG-100 stearate, phenylethyl resorcinol, tocopheryl acetate, C13-14 isoparaffin, disodium EDTA, laureth-7, allantoin, aloe barbadensis leaf juice, hydrogenated lecithin, ethylhexylglycerin, glycyrrhiza glabra (licorice) root extract

### Production Example 266: Salicyclic Acid Lotion

ACTIVE INGREDIENT: Salicylic Acid 1%
INACTIVE INGREDIENTS: water, helianthus annuus (sunflower) seed oil, glycerin, stearic acid, sucrose polystearate, salix nigra (willow) bark extract, beeswax, cymbopogon schoenanthus extract, Hamamelis virginiana (witch hazel) leaf extract, humulus lupulus (hops) extract, equisetum hiemale extract, hydrastis canadensis (goldenseal) extract, epilobium fleischeri extract, lecithin, chondrus crispus (carageenan), fragrance, glucose, xanthan gum, sucrose, sucrose stearate, potassium sorbate, sodium chloride, glucose oxidase, lactoperoxidase, phenoxyethanol.

### Production Example 267: Salicyclic Acid Cream

ACTIVE INGREDIENT: Salicylic Acid 2%
INACTIVE INGREDIENTS: water, caprylic/capric triglyceride, glycerin, salix nigra (willow) bark extract, stearyl alcohol, cetyl alcohol, glyceryl stearate, beeswax, calophyllum inophyllum seed oil, cymbopogon schoenanthus extract, dipotassium glycyrrhizate, equsetum hiemale extract, Hamamelis virginiana (witch hazel) leaf extract, humulus lupulus (hops) extract, hydrastis canadensis (goldenseal) extract, lonicera caprifulium extract, lonicera japonica extract, simmondsia chinensis (jojoba) seed oil, stearic acid, sorbic acid, tocopherol, xanthan gum, lecithin, glucose, glycine soja (soybean) oil, fragrance, sodium chloride, glucose oxidase, lactoperoxidase, phenoxyethanol.

### Production Example 268: Salicyclic Acid Emulsion

ACTIVE INGREDIENT: Salicylic Acid 1%
INACTIVE INGREDIENTS: water, hydrogenated jojoba oil, stearic acid, crambe abyssinica seed oil, glycerin, vitis vinifera) grape seed oil, cetyl alcohol, salix nigra (willow) bark extract, cocas nucifera (coconut) oil, beeswax, butyrospermum parkii (shea) butter, Vaccinium myrtillus fruit extract, sacchrum officinarum (sugar cane) extrace, Citrus aurantium dulcis (orange) fruit extrace, citrus medica limonum (lemon) fruit extract, Acer saccharum (sugar maple) extract, tocopherol, sucrose stearate, xanthan gum, citric acid, fragrance, phenoxyethanol.

### Production Example 269: Salicyclic Acid Kit

ACTIVE INGREDIENT: Salicylic Acid 1%
INACTIVE INGREDIENTS: water, helianthus annuus (sunflower) seed oil, glycerin, stearic acid, sucrose polystearate, salix nigra (willow) bark extract, beeswax, cymbopogon schoenanthus extract, Hamamelis virginiana (witch hazel) leaf extract, humulus lupulus (hops) extract, equisetum hiemale extract, hydrastis canadensis (goldenseal) extract, epilobium fleischeri extract, lecithin, chondrus crispus (carageenan), fragrance, glucose, xanthan gum, sucrose, sucrose stearate, potassium sorbate, sodium chloride, glucose oxidase, lactoperoxidase, phenoxyethanol.

### Production Example 270: Salicyclic Acid Liquid

ACTIVE INGREDIENT: Salicylic Acid 1%
INACTIVE INGREDIENTS: aqua (water, eau), alcohol denat., glycerin, salix nigra (willow) bark extract, cymbopogon schoenanthus extract, equisetum hiemale extract, parfum (fragrance), Hamamelis virginiana (witch hazel) leaf extract, humulus lupulus (hops) extract, hydrastis canadensis (goldenseal) extract, glucose, sodium chloride, glucose oxidase, lactoperoxidase

### Production Example 271: Salicyclic Acid Lotion

ACTIVE INGREDIENT: Salicylic Acid 1.5%
INACTIVE INGREDIENTS: alcohol denat., water, methylpropanediol, glycolic acid, diisopropyl adipate, octadecenedioic acid, hydroxethylpiperazine ethane sulfonic acid, citric acid, capryloyl salicylic acid, sodium hydroxide, polyquaternium-10

### Production Example 272: Salicyclic Acid and Benzoyl Peroxide Kit

ACTIVE INGREDIENT: Salicylic Acid 2%
INACTIVE INGREDIENTS: water, PPG-15 stearyl ether, glycerin, stearyl alcohol, cetyl betaine, distearyldimonium chloride, sodium lauryl sulfate, oxidized polyethylene, cetyl alcohol, alcohol denat., steareth-21, sodium chloride, behenyl alcohol, PPG-30, steareth-2, fragrance, menthol, disodium EDTA, BHT

### Production Example 273: Salicyclic Acid and Benzoyl Peroxide Kit

ACTIVE INGREDIENT: Salicylic Acid 2%
INACTIVE INGREDIENTS: water, PPG-15 stearyl ether, glycerin, stearyl alcohol, cetyl betaine, distearyldimonium chloride, sodium lauryl sulfate, oxidized polyethylene, cetyl alcohol, alcohol denat., steareth-21, sodium chloride, behenyl alcohol, PPG-30, steareth-2, fragrance, menthol, disodium EDTA, BHT

### Production Example 274: Salicyclic Acid and Benzoyl Peroxide Kit

ACTIVE INGREDIENT: Salicylic Acid 2%
INACTIVE INGREDIENTS: Water, PPG-15 Stearyl Ether, Glycerin, Stearyl Alcohol, Cetyl Betaine, Distearyldimonium Chloride, Sodium Lauryl Sulfate, Oxidized Polyethylene, Cetyl Alcohol, Alcohol, Steareth-21, Sodium Chloride, Behenyl Alcohol, PPG-30, Steareth-2, Fragrance, Menthol, Disodium EDTA, BHT.

### Production Example 275: Salicyclic Acid and Benzoyl Peroxide Kit

ACTIVE INGREDIENT: Salicylic Acid 2%
INACTIVE INGREDIENTS: water, sodium cocoyl isethionate, cetearyl alcohol, glycerin, sodium laureth sulfate, sodium cocoamphoacetate, cocamidopropyl betaine, sodium hydroxide, acrylates/C10-30 alkyl acrylate crosspolymer, fragrance, disodium EDTA, Lavandula stoechas extract, Helichrysum italicum extract, Cistus monspeliensis extract

### Production Example 276: Sulfur Gel

ACTIVE INGREDIENT: Sulfur 6%
INACTIVE INGREDIENTS: water, dicaprylyl carbonate, montmorillonite, alcohol denat., neopentyl glycol diheptanoate, glycerin, acacia senegal gum, triethanolamine, olive oil PEG-7 esters, butylene glycol, camellia sinensis leaf extract, cymbopogon schoenanthus extract, geranium maculatum extract, melaleuca alternifolia (tea tree) leaf oil, dipotassium glycyrrhizate, hydroxyphenyl propamidobenzoic acid, tocopheryl acetate, allantoin, pentylene glycol, ethylhexylglycerin, butyl oleate, glyceryl acrylate/acrylic acid copolymer, acrylates/C10-30 alkyl acrylate crosspolymer, ethoxydiglycol, disodium EDTA, butylparaben, ethylparaben, isobutylparaben, methylparaben, phenoxyethanol, propylparaben, fragrance.

### Production Example 277: Sulfur Lotion

ACTIVE INGREDIENT: Sulfur 8%
INACTIVE INGREDIENTS: water, octyldodecanol, tricaprylin, sorbitol, stearic acid, magnesium aluminum silicate, glyceryl polymethacrylate, glycol stearate, propylene glycol, cyclopentasiloxane, cyclohexasiloxane, stearyl alcohol, ceteareth-20, xanthan gum, talc, triethanolamine, imidazolidinyl urea, methylparaben, propylparaben, fragrance, iron oxides, titanium dioxide.

### Production Example 278: Sulfur Lotion

ACTIVE INGREDIENT: Sulfur 6%
INACTIVE INGREDIENTS: water, kaolin, glycereth-26, linoleic acid, cetearyl alcohol, magnesium aluminum silicate, ceteareth-20, sodium lauroyl sarcosinate, linolenic acid, tocopheryl acetate, tocopherol, butylene glycol, melaleuca alternifolia (tea tree) leaf oil, cymbopogon schoenanthus extract, geranium maculatum extract, hydrated silica, ethoxydiglycol, methylparaben, ethylparaben, fragrance, titanium dioxide, zinc oxide

### Production Example 279: Sulfur Lotion

ACTIVE INGREDIENT: Sulfur 6%
INACTIVE INGREDIENTS: water, kaolin, glycereth-26, linoleic acid, cetearyl alcohol, magnesium aluminum silicate, synthetic wax, ceteareth-20, sodium lauroyl sarcosinate, linolenic acid, tocopherol, tocopheryl acetate, melaleuca alternifolia (tea tree) leaf oil, cymbopogon schoenanthus extract, geranium maculatum extract, hydrated silica, ethoxydiglycol, tetrasodium EDTA, sodium sulfate, butylene glycol, phenoxyethanol, fragrance, titanium dioxide, zinc oxide, chromium hydroxide green

### Production Example 280: Sulfur and Resorcinol Cream

ACTIVE INGREDIENT: Resorcinol 2%
INACTIVE INGREDIENTS: Water, Alcohol Denat., Bentonite, Glyceryl Stearate SE, Propylene Glycol, Isopropyl Myristate, Fragrance, Simethicone, Methylparaben, Propylparaben, Titanium Dioxide, Iron Oxides

### Production Example 281: Adapalene Cream

ACTIVE INGREDIENTS: Adapalene 1 mg in 1 g
INACTIVE INGREDIENTS: carbomer homopolymer type b (allyl pentaerythritol or allyl sucrose crosslinked), cyclomethicone, edetate disodium, methyl glucose sesquistearate, glycerin, methylparaben, peg-20 methyl glucose sesquistearate, phenoxyethanol, propylparaben, water, squalane, trolamine.

### Production Example 282: Adapalene Gel

ACTIVE INGREDIENT: Adapalene 0.1% (retinoid)
INACTIVE INGREDIENTS: carbomer 940, edetate disodium, methylparaben, poloxamer 182, propylene glycol, purified water and sodium hydroxide.
May contain hydrochloric acid to adjust pH

### Production Example 283: Adapalene Gel

ACTIVE INGREDIENT: Adapalene 3 mg in 1g
INACTIVE INGREDIENTS: Carbomer Homopolymer Type C, Edetate Disodium, Propylene Glycol, Water, Sodium Hydroxide, Hydrochloric Acid, Poloxamer 124.

### Production Example 284: Adapalene Gel

ACTIVE INGREDIENT: Adapalene 1 mg in 1 g
INACTIVE INGREDIENTS; carbomer 940, edetate disodium, methylparaben, poloxamer 182, propylene glycol, water, sodium hydroxide, hydrochloric acid.

### Production Example 285: Adapalene Lotion

ACTIVE INGREDIENT: Adapalene 0.1 g in 100 mL
INACTIVE INGREDIENTS: carbomer homopolymer type a, edetate disodium, medium-chain triglycerides, methylparaben, phenoxyethanol, poloxamer 124, polyethylene glycol 300, polyethylene glycol 1500, propylene glycol, propylparaben, sodium hydroxide, stearyl alcohol, water.

### Production Example 286: Adapalene Cream

ACTIVE INGREDIENT: Adapalene 1 mg in 1 g
INACTIVE INGREDIENTS: glycerin, edetate disodium, methylparaben, phenoxyethanol, water, squalane, trolamine.

### Production Example 287: Adapalene Gel

ACTIVE INGREDIENT: Adapalene 3 mg in 1 g
INACTIVE INGREDIENTS: Carbomer Homopolymer Type C, Edetate Disodium, Methylparaben, Propylene Glycol, Water, Sodium Hydroxide, Hydrochloric Acid, Poloxamer 124.

### Production Example 288: Adapalene Gel

ACTIVE INGREDIENT: Adapalene 1 mg in 1 g
INACTIVE INGREDIENT: Carbomer Homopolymer Type C, Edetate disodium, Methylparaben, Poloxamer 182, Propylene Glycol, Water, Sodium hydroxide, Hydrochloric acid.

### Production Example 289: Adapalene Lotion

ACTIVE INGREDIENT: Adapalene 1 mg in 1 g
INACTIVE INGREDIENTS: Carbomer Homopolymer Type A, Edetate Disodium, Medium-chain Triglycerides, Methylparaben, Phenoxyethanol, Poloxamer 124, Propylene Glycol, Propylparaben, Sodium Hydroxide, Stearyl Alcohol, Water.

### Production Example 290: Adapalene Gel

ACTIVE INGREDIENT: Adapalene 0.1% (retinoid)
INACTIVE INGREDIENTS: carbomer 940, edetate disodium, methylparaben, poloxamer 182, propylene glycol, purified water and sodium hydroxide. May contain hydrochloric acid to adjust pH.

### Production Example 291: Adapalene and Benzoyl Peroxide Gel

ACTIVE INGREDIENT: Adapalene 1 mg in 1 g; Benzoyl Peroxide 25 mg in 1 g
INACTIVE INGREDIENTS: carbomer homopolymer type c, docusate sodium, edetate disodium, glycerin, poloxamer 182, polysorbate 80, propylene glycol, water, sorbitan monooleate, xanthan gum.

### Production Example 292: Adapalene and Benzoyl Peroxide Gel

ACTIVE INGREDIENT: Adapalene 0.1 g in 100 g; Benzoyl Peroxide 2.5 mg in 100 g
INACTIVE INGREDIENTS: docusate sodium, edetate disodium, glycerin, isohexadecane, polysorbate 80, propylene glycol, water, sorbitan monooleate, poloxamer 124.

### Production Example 293: Adapalene and Benzoyl Peroxide Gel

ACTIVE INGREDIENT: Adapalene 1 mg in 1 g; Benzoyl Peroxide 25 mg in 1 g
INACTIVE INGREDIENTS: docusate sodium, edetate disodium, glycerin, isohexadecane, poloxamer 124, polysorbate 80, propylene glycol, water, sorbitan monooleate.

### Production Example 294: Adapalene and Benzoyl Peroxide Gel

ACTIVE INGREDIENT: Adapalene 1 mg in 1 g; Benzoyl Peroxide 25 mg in 1 g
INACTIVE INGREDIENTS: carbomer homopolymer type c, docusate sodium, edetate disodium, glycerin, limonene, (+)-, poloxamer 182, polysorbate 80, propylene glycol, water, sodium hydroxide, sorbitan monooleate.

### Production Example 295: Adapalene and Benzoyl Peroxide Gel

ACTIVE INGREDIENT: Adapalene 1 mg in 1 g; Benzoyl Peroxide 25 mg in 1 g
INACTIVE INGREDIENTS; docusate sodium, edetate disodium, glycerin, poloxamer 124, sodium acryloyldimethyltaurate-acrylamide copolymer (1:1; 90000-150000 mpa·s), propylene glycol, water.

### Production Example 296: Adapalene and Benzoyl Peroxide Gel

ACTIVE INGREDIENT: Adapalene 1 mg in 1 g; Benzoyl Peroxide 25 mg in 1 g
INACTIVE INGREDIENTS: docusate sodium, edetate disodium, glycerin, Isohexadecane, poloxamer 124, polysorbate 80, propylene glycol, Water, Sorbitan Monooleate.

### Production Example 297: Adapalene and Benzoyl Peroxide Gel

ACTIVE INGREDIENT: Adapalene 3 mg in 1 g; Benzoyl Peroxide 25 mg in 1 g
INACTIVE INGREDIENTS: acrylic acid/sodium acrylate copolymer (1:1; 600 mpa·s at 0.2%), docusate sodium, edetate disodium, glycerin, isohexadecane, poloxamer 124, polysorbate 80, propylene glycol, water, sorbitan monooleate.

### Production Example 298: Adapalene and Benzoyl Peroxide Gel

ACTIVE INGREDIENT: Adapalene 1 mg in 1 g; Benzoyl Peroxide 25 mg in 1 g
INACTIVE INGREDIENTS: docusate sodium, edetate disodium, glycerin, Isohexadecane, poloxamer 124, polysorbate 80, propylene glycol, Water, Sorbitan Monooleate.

### Production Example 299: Tretinoin Gel

ACTIVE INGREDIENT: Tretinoin Gel, USP 0.05%
INACTIVE INGREDIENTS: benzyl alcohol, butyl paraben, butylated hydroxytoluene, carbomer 980 ethyl paraben, fish collagen hydrolyzates, glycerin, iso-butyl paraben, methylparaben, octoxynol 9, phenoxyethanol, propylparaben, purified water, sodium hyaluronate, and trolamine.

### Production Example 300: Tretinoin Gel

ACTIVE INGREDIENT: Tretinoin 1 mg in 1 g
INACTIVE INGREDIENTS: water, carbomer homopolymer type b (allyl pentaerythritol crosslinked), glycerin, edetate disodium, propylene glycol, sorbic acid, ppg-20 methyl glucose ether distearate, cyclomethicone, benzyl alcohol, trolamine, butylated hydroxytoluene.

### Production Example 301: Dapsone Gel

ACTIVE INGREDIENT: Dapsone 50 mg in 1 g
INACTIVE INGREDIENTS: carbomer homopolymer type c (allyl pentaerythritol crosslinked), diethylene glycol monoethyl ether, methylparaben, water, sodium hydroxide.

### Production Example 302: Dapsone Gel

ACTIVE INGREDIENT: Dapsone 50 mg in 1 g
INACTIVE INGREDIENTS: carbomer homopolymer type C, diethylene glycol monoethyl ether, methylparaben, sodium hydroxide, water.

### Production Example 303: Dapsone Gel

ACTIVE INGREDIENT: Dapsone 75 mg in 1 g
INACTIVE INGREDIENTS: Carbomer Homopolymer Type C (Allyl Pentaerythritol Crosslinked), diethylene glycol monoethyl ether, light mineral oil, methylparaben, polysorbate 80, water, sodium hydroxide, sorbitol monooleate.

### Production Example 304: Dapsone Gel

ACTIVE INGREDIENT: Dapsone 50 mg in 1 g
INACTIVE INGREDIENTS; Carbomer Homopolymer Type C (Allyl Pentaerythritol Crosslinked), diethylene glycol monoethyl ether, methylparaben, water, sodium hydroxide.

### Production Example 305: Dapsone Gel

ACTIVE INGREDIENT; Dapsone 50 mg in 1 g
INACTIVE INGREDIENTS: carbomer homopolymer type c, diethylene glycol monoethyl ether, methylparaben, sodium hydroxide, water.

### Production Example 306: Dapsone Gel

ACTIVE INGREDIENT: Dapsone 75 mg in 1 g
INACTIVE INGREDIENTS: diethylene glycol monoethyl ether, methylparaben, isohexadecane, polysorbate 80, water.

### Production Example 307:

ACTIVE INGREDIENTS (external anti-acne agents)
ACTIVE INGREDIENTS (external anti-acne agents) suitable for formulation with: (1) suitable inactive ingredients (excipients), and (2) a cannabinoid, terpene, flavonoid, or combination thereof, to provide various topical dosage forms (e.g., creams, gels, lotions, etc.).

TABLE 1

| Active | Amount |
|---|---|
| Benzoyl peroxide | 10 wt. % |
| Benzoyl peroxide | 2.50 wt. % |
| Benzoyl peroxide | 5.00 wt. % |
| Benzoyl peroxide | 3.50 wt. % |
| Benzoyl peroxide | 3.70 wt. % |
| Benzoyl peroxide | 7 wt. % |
| Benzoyl peroxide | 6 wt. % |
| Benzoyl peroxide | 8.50 wt. % |
| Benzoyl peroxide | 3 wt. % |
| Salicylic acid | 2 wt. % |
| Salicylic acid | 1 wt. % |
| Salicylic acid | 1.90 wt. % |
| Salicylic acid | 0.50 wt. % |
| Salicylic acid | 1.50 wt. % |
| Salicylic acid | 0.60 wt. % |
| Sulfur | 10 wt. % |
| Sulfur | 6 wt. % |
| Sulfur | 8 wt. % |
| Resorcinol | 2 wt. % |
| Adapalene | 1 mg in 1 g |
| Adapalene | 0.10 wt. % |
| Adapalene | 3 mg in 1 g |
| Adapalene | 0.1 g in 100 mL |
| Adapalene/Benzoyl Peroxide | 1 mg in 1 g/25 mg in 1 g |
| Adapalene/Benzoyl Peroxide | 0.1 g in 100 g/2.5 mg in 100 g |
| Adapalene/Benzoyl Peroxide | 3 mg in 1 g/25 mg in 1 g |
| Tretinoin | 1 mg in 1 g |
| Tretinoin | 0.05 wt. % |
| Dapsone | 50 mg in 1 g |
| Dapsone | 75 mg in 1 g |
| Glycolic acid | 10 wt. % |
| Glycolyic acid | 7 wt. % |

TABLE 2

| ACTIVE | DOSAGE FORM & STRENGTHS |
|---|---|
| Minocycline | Topical foam: 4 wt. % |
| Clindamycin phosphate | Solution or lotion: 10 mg per mL |
| | Gel: 10 mg per gram |
| Clindamycin phosphate | Gel: 1 wt. % or 10 mg per gram |
| Clindamycin phosphate | Solution: 1 wt. % or 10 mg per mL |
| Clindamycin phosphate | Solution: 1 wt. % or 10 mg per mL |
| Clindamycin phosphate | Solution: 1 wt. % or 10 mg per mL |
| Clindamycin phosphate | Solution: 1 wt. % or 10 mg per mL |
| Clindamycin phosphate | Solution: 1 wt. % or 10 mg per mL |
| Clindamycin phosphate | Solution: 1 wt. % w/v |
| Clindamycin phosphate | Foam: 1 wt. % or 10 mg per gram |
| Tretinoin (retinoid) | Cream (Emollient): 0.05 wt. % |
| Tretinoin | Gel microsphere: 0.1 wt. %, 0.08 wt. %, |
| | 0.04 wt. % |
| Tretinoin | Cream: 0.02 wt. % |
| Tretinoin | Gel: 0.05 wt. % |
| Tretinoin | Lotion: 0.05 wt. % |
| Tretinoin | Gel: 0.025 wt. % |
| Tretinoin | Cream or Gel: 0.01 wt. %, 0.025 wt. %, or |
| | 0.05 wt. % w/w |
| Tretinoin | Cream: 0.1 wt. % |
| Tazarotene | Cream: 0.1 wt. % (retinoid) |
| Tazarotene | Lotion: 0.045 wt. % |
| Tazarotene | Foam: 0.1 wt. % |
| Tazarotene | Gel: 0.05 wt. % or 0.1 wt. % |
| Adapalene/benzoyl peroxide | Gel: 1 mg (0.1 wt. %) adapalene and 25 mg |
| | (2.5 wt. %) benzoyl peroxide |
| Adapalene/benzoyl peroxide | Gel: 0.3 wt. % adapalene/2.5 wt. % benzoyl |
| | peroxide in 15 g, 30 g, 45 g, 60 g and 70 g pumps |
| dapsone | Gel: 7.5 wt. % |
| Sulfacetamide sodium | Lotion: 100 mg/mL |
| Sulfamethoxazole/Trimethoprim | Suspension: 40 mg/mL Sulf/8 mg/mL Trim |
| Sulfamethoxazole/Trimethoprim | Suspension: 40 mg/mL Sulf/8 mg/mL Trim |
| Cephalexin | Suspesion: 25 mg/mL, 50 mg/mL |
| Erythromycin | Medicated pad: 20 mg/mL |
| Erythromycin | Gel: 0.02 mg/mg gel |
| Erythromycin | Medicated pad: 2 wt. % |
| Erythromycin | Solution: 20 mg/mL |
| Erythromycin | Ointment: 0.5 wt. % |
| Erythromycin | Solution: 20 mg/mL |
| Benzoyl peroxide/Clindamycin phosphate | Gel: 0.05 mg/mg BP/0.012 mg/mg CP |
| Benzoyl peroxide/Clindamycin phosphate | Gel: 0.05 mg/mg BP/0.012 mg/mg CP |
| Benzoyl peroxide/Clindamycin phosphate | Gel: 0.025 mg/mg BP/0.012 mg/mg CP |
| Benzoyl peroxide/Clindamycin phosphate | Gel: 0.05 mg/mg BP/0.01 mg/mg CP |
| Benzoyl peroxide/Clindamycin phosphate | Gel: 0.0375 mg/mg BP/0.012 mg/mg CP |
| Benzoyl peroxide/Clindamycin phosphate | Gel: 5 wt. % BP/1.2 wt. % CP |
| Benzoyl peroxide/Clindamycin phosphate | Gel: 5 wt. % BP/1.2 wt. % CP |
| Benzoyl peroxide/erythromycin | Gel: 0.05 mg/mg BP/0.03 mg/mg Eryth |
| Benzoyl peroxide/erythromycin | Gel: 5 wt. % BP/3 wt. % Eryth |
| Benzoyl peroxide/erythromycin | Gel: 0.05 mg/mg BP/0.03 mg/mg Eryth |
| Clindamycin/Tretinoin | Gel: 0.012 mg/mg Clin/0.00025 mg/mg Tret |
| Clindamycin/Tretinoin | Gel: 0.012 mg/mg Clin/0.00025 mg/mg Tret |
| Retapamulin | Ointment: 0.01 mg/mg |
| azelaic acid | Cream: 200 mg/mL |
| azelaic acid | Gel: 0.15 mg/mg |
| | Foam: 150 mg/mL |
| benzoyl peroxide/sulfur | Gel: 0.06 mg/mg BP/0.03 mg/mg Sulfur |
| benzoyl peroxide/sulfur | Gel: 6 wt. % BP/3 wt. % Sulfur |
| Sulfacetamide sodium/sulfur | Liquid soap: 100 mg/mL SS/50 mg/mL Sulfur |
| | 2) 98 mg/mL SS/48 mg/mL Sulfur |
| | Medicated Pad: 100 mg/mL SS/50 mg/mL |
| | Sulfur 2) 98 mg/mL SS/48 mg/mL Sulfur |
| | Cream: 100 mg/mL SS/50 mg/mL Sulfur |
| | Lotion: 1) 100 mg/mL SS/50 mg/mL Sulfur 2) |
| | 98 mg/mL SS/48 mg/mL Sulfur |
| Sulfacetamide sodium/sulfur | Suspension: 8 wt. % SS/4 wt. % Sulfur |
| Sulfacetamide sodium/sulfur | Medicated liquid soap: 90 mg/mL SS/ |
| | 40 mg/mL Sulfur |
| Sulfacetamide sodium/sulfur | Foam: 95 mg/mL SS/50 mg/mL Sulfur |
| | Medicated Liquid Soap: 1) 95 mg/mL SS/ |
| | 50 mg/mL Sulfur 2) 100 mg/mL SS/50 mg/mL |
| | Sulfur 3) 100 mg/mL SS/20 mg/mL Sulfur |
| | Gel: 0.1 mg/mg SS/0.05 mg/mg Sulfur |
| | Cream: 1) 100 mg/mL SS/50 mg/mL Sulfur 2) |
| | 100 mg/mL SS/20 mg/mL Sulfur |
| | Medicated Pad: 1) 95 mg/mL SS/50 mg/mL |
| | Sulfur 2) 100 mg/mL SS/20 mg/mL Sulfur |
| Sulfacetamide sodium/sulfur | Cream: 10 wt. % SS/5 wt. % sulfur |
| Sulfacetamide sodium/sulfur | Emollient Cream: 10 wt. % SS/5 wt. % sulfur |
| Sulfacetamide sodium/sulfur | Cream: 10 wt. % SS/2 wt. % sulfur |
| Sulfacetamide sodium/sulfur | Medicated Liquid Soap: 10 wt. % SS/5 wt. % |
| | sulfur or 100 mg/mL SS/50 mg/mL |
| Sulfacetamide sodium/sulfur | Foam: 10 wt. % SS/2 wt. % sulfur |
| Sulfacetamide sodium/sulfur | Medicated Liquid Soap: 10 wt. % SS/2 wt. % |
| | sulfur or 100 mg/mL SS/20 mg/mL sulfur |
| Sulfacetamide sodium/sulfur | Medicated Liquid Soap: 100 mg/mL SS/ |
| | 10 mg/mL Sulfur |
| Sulfacetamide sodium/sulfur | Medicated Liquid Soap: 100 mg/mL SS/ |
| | 10 mg/mL Sulfur |
| Sulfacetamide sodium/sulfur | Foam: 100 mg/mL SS/50 mg/mL Sulfur |
| Sulfacetamide sodium/sulfur | Cream: 100 mg/mL SS/50 mg/mL Sulfur |
| Sulfacetamide sodium/sulfur | Solution: 10 wt. % SS/5 wt. % sulfur |
| Sulfacetamide sodium/sulfur | Cream: 98 mg/mL SS/48 mg/mL Sulfur or 10 |
| | wt. % SS/5 wt. % sulfur |
| Sulfacetamide sodium/sulfur | Cloths: 10 wt. % SS/5 wt. % sulfur |
| Sulfacetamide sodium/sulfur | Cleanser/Cream: 10 wt. % SS/5 wt. % sulfur |
| Sulfacetamide sodium/sulfur | Suspension: 10 wt. % SS/5 wt. % sulfur |
| Sulfacetamide sodium/sulfur | Medicated Liquid Soap: 100 mg/mL SS/ |
| | 50 mg/mL Sulfur |
| | Medicated Pad: 100 mg/mL SS/50 mg/mL Sulfur |
| | Lotion: 100 mg/mL SS/50 mg/mL Sulfur |
| | Cream: 100 mg/mL SS/50 mg/mL Sulfur |
| Sulfacetamide sodium/sulfur | Cloths: 10 wt. % SS/5 wt. % sulfur |
| Sulfacetamide sodium/sulfur | Cream: 10 wt. % SS/5 wt. % sulfur |
| Sulfacetamide sodium/sulfur | Cream: 100 mg/mL SS/50 mg/mL Sulfur |
| | Medicated Liquid Soap: 100 mg/mL SS/ |
| | 10 mg/mL Sulfur |
| Sulfacetamide sodium/sulfur | Medicated Liquid Soap: 100 mg/mL SS/ |
| | 50 mg/mL Sulfur |
| Sulfacetamide sodium | Medicated Liquid Soap: 100 mg/mL |
| Sulfacetamide sodium/sulfur | Medicated Liquid Soap: 100 mg/mL SS/ |
| | 50 mg/mL Sulfur |
| Sulfacetamide sodium/sulfur | Lotion: 100 mg/mL SS/50 mg/mL Sulfur |
| | Gel: 0.1 mg/mg SS/0.05 mg/mg Sulfur |
| | Medicated pad: 100 mg/mL SS/50 mg/mL |
| | Sulfur |
| | Foam: 100 mg/mL SS/40 mg/mL Sulfur |
| | Medicated liquid soap: 1) 100 mg/mL SS/ |
| | 40 mg/mL Sulfur 2) 100 mg/mL SS/50 mg/mL |
| | Sulfur 3) 100 mg/mL SS/45 mg/mL Sulfur |
| Sulfacetamide sodium/sulfur | Suspension: 10 wt. % SS/4.5 wt. % sulfur |
| Sulfacetamide sodium/sulfur | Cream: 10 wt. % SS/5 wt. % sulfur |
| Sulfacetamide sodium/sulfur | Foam: 10 wt. % SS/4 wt. % sulfur |
| Sulfacetamide sodium/sulfur | Emollient foam: 10 wt. % SS/5 wt. % sulfur |
| Sulfacetamide sodium/sulfur | Gel: 0.1 mg/mg SS/0.05 mg/mg Sulfur |
| | Lotion: 100 mg/mL SS/50 mg/mL Sulfur |
| | Cream: 100 mg/mL SS/50 mg/mL Sulfur |
| | Medicated Liquid Soap: 100 mg/mL SS/ |
| | 50 mg/mL Sulfur |
| | Medicated pad: 100 mg/mL SS/40 mg/mL |
| | Sulfur |
| Sulfacetamide sodium/sulfur | Cream: 10 wt. % SS/5 wt. % sulfur |
| Sulfacetamide sodium/sulfur | Cream: 10 wt. % SS/5 wt. % sulfur |
| Sulfacetamide sodium/sulfur | Medicated Liquid Soap: 90 mg/mL SS/ |
| | 45 mg/mL Sulfur |
| Sulfacetamide sodium/sulfur | Medicated liquid soap: 80 mg/mL SS/ |
| | 40 mg/mL Sulfur; 90 mg/mL SS/40 mg/mL |
| | Sulfur |
| Sulfacetamide sodium/sulfur | Medicated pad: 100 mg/mL SS/40 mg/mL |
| | Sulfur |
| Sulfacetamide sodium/sulfur | Suspension: 8 wt. % SS/4 wt. % sulfur |
| Sulfacetamide sodium/sulfur | Cream: 100 mg/mL SS/50 mg/mL Sulfur |
| Sulfacetamide sodium/sulfur | Cream: 100 mg/mL SS/50 mg/mL Sulfur |
| Sulfacetamide sodium/sulfur | Emollient cream: 10 wt. % SS/5 wt. % sulfur |
| Trifarotene | Cream: 0.0005 wt. % |
| Benzoyl peroxide/hydrocortisone | Lotion: 5 wt. % BP/0.5 wt. % HC |
| Benzoyl peroxide/hydrocortisone | Lotion: 5 wt. % BP/0.5 wt. % HC |
| Benzoyl peroxide | Lotion: 50 mg/mL |
| Benzoyl peroxide/hydrocortisone | Lotion: 50 mg/mL BP/5 mg/mL HC |
| Benzoyl peroxide/salicylic acid | Pad: 5 wt. % BP/2 wt. % SA |
| Benzoyl peroxide | Medicated pad: 80 mg/mL or 40 mg/mL |
| Benzoyl peroxide/salicylic acid | Medicated pad: 4 wt. % BP/1 wt. % SA |
| Benzoyl peroxide/salicylic acid | Medicated pad: 80 mg/mL BP/20 mg/mL SA |
| Benzoyl peroxide | Foam: 9.8 wt. % |
| Metronidazole | Cream: 0.75 wt. % |
| Metronidazole | Gel: 0.75 wt. %, 1 wt. % |
| Metronidazole | Lotion: 0.75 wt. % |
| Metronidazole | Cream: 1 wt. % |
| Metronidazole | Cream: 0.75 wt. % |
| Metronidazole | Cream/Gel: 0.75 wt. % |
| Metronidazole | Cream: 0.75 wt. % |
| Benzoyl peroxide | Gel: 0.025 mg/mg or 0.05 mg/mg |
| Benzoyl peroxide | Lotion: 100 mg/mL |
| Benzoyl peroxide | Cream: 100 mg/mL |
| | Gel: 0.1 mg/mg |
| Benzoyl peroxide | Lotion: 5 wt. % |
| Benzoyl peroxide | Gel: 5 wt. % or 10 wt. % |
| Benzoyl peroxide | Gel: 2.75 wt. % or 5.25 wt. % |
| Benzoyl peroxide | Medicated liquid soap: 25 mg/mL, 26 mg/mL, |
| | 50 mg/mL, 100 mg/mL |
| | Gel: 0.025 mg/mg, 0.1 mg/mg, 0.05 mg/mg |
| | Cream: 50 mg/mL |
| Benzoyl peroxide | Gel: 2.5 wt. %, 5 wt. % or 10 wt. % |
| Benzoyl peroxide | Solution: 5 wt. % |
| Benzoyl peroxide | Solution: 5 wt. % or 10 wt. % |
| Benzoyl peroxide | Gel: 0.05 mg/mg, 0.1 mg/mg |
| Benzoyl peroxide | Gel: 0.1 mg/mg |
| Benzoyl peroxide | Cream: 50 mg/mL, 100 mg/mL |
| Benzoyl peroxide | Foam: 53 mg/mL, 98 mg/mL |
| Benzoyl peroxide | Medicated Pad: 60 mg/mL |
| | Medicated liquid soap: 70 mg/mL |
| Benzoyl peroxide | Gel: 0.0525 mg/mg |
| Benzoyl peroxide | Medicated liquid soap: 52.5 mg/mL |
| Benzoyl peroxide | Gel: 0.0275 mg/mg |
| Benzoyl peroxide | Lotion: 75 mg/mL |
| | Medicated pad: 75 mg/mL |
| Benzoyl peroxide | Foam: 5 wt. % |
| Benzoyl peroxide | Foam: 10 wt. % |
| Benzoyl peroxide | Cloth: 3 wt. % |
| Benzoyl peroxide | Cloth: 6 wt. % |
| Benzoyl peroxide | Cloth: 9 wt. % |
| Benzoyl peroxide | Gel: 4 wt. %, 8 wt. % |
| Benzoyl peroxide | Emulsion: 7 wt. % |
| Benzoyl peroxide | Lotion: 40 mg/mL, 80 mg/mL |
| | Gel: 0.08 mg/mg, 0.04 mg/mg |
| | Medicated liquid soap: 40 mg/mL, 80 mg/mL |
| Benzoyl peroxide | Creamy Wash: 4 wt. % or 8 wt. % BP with |
| | Cleansing bar: 5 wt. % BP |
| Benzoyl peroxide | Creamy Wash: 4 wt. % or 8 wt. % |
| Benzoyl peroxide | Cream: 100 mg/mL |
| Benzoyl peroxide | Lotion: 35 mg/mL |
| | Medicated liquid soap: 35 mg/mL |
| Benzoyl peroxide | Gel: 0.07 mg/mg |
| Benzoyl peroxide | Medicated liquid soap: 50 mg/mL, |
| | 100 mg/mL |
| | Medicated bar soap: 0.1 mg/mg |
| Benzoyl peroxide | Medicated liquid soap: 100 mg/mL |
| | Gel: 0.1 mg/mg |
| | Medicated Bar Soap: 0.1 mg/mg |
| Benzoyl peroxide | Cream: 35 mg/mL, 55 mg/mL, 85 mg/mL |
| Benzoyl peroxide | Cream: 5.5 wt. % |
| Benzoyl peroxide | Medicated liquid soap: 70 mg/mL |
| Benzoyl peroxide | Cream: 3.5 wt. % |
| Benzoyl peroxide | Gel: 10 wt. % |
| Benzoyl peroxide | Medicated pad: 30 mg/mL, 60 mg/mL, |
| | 90 mg/mL |
| | Medicated liquid soap: 30 mg/mL, 60 mg/mL, |
| | 90 mg/mL |
| Benzoyl peroxide | Cream: 10 wt. % |
| Benzoyl peroxide | Cream: 10 wt. % |
| Benzoyl peroxide | Medicated pad: 42.5 mg/mL, 70 mg/mL |
| | Medicated liquid soap: 42.5 mg/mL, |
| | 70 mg/mL |
| Benzoyl peroxide | Cream: 50 mg/mL, 30 mg/mL |
| | Gel: 0.1 mg/mg, 0.025 mg/mg, 0.05 mg/mg |
| | Foam: 100 mg/mL |
| | Medicated bar soap: 0.1 mg/mg, 0.05 mg/mg |
| | Medicated liquid soap: 25 mg/mL, 40 mg/mL, |
| | 80 mg/mL, 100 mg/mL |
| Benzoyl peroxide | Cream: 4 wt. % |
| Benzoyl peroxide | Medicated liquid soap: 85 mg/mL, 65 mg/mL, |
| | 45 mg/mL, 70 mg/mL |
| | Gel: 0.065 mg/mg, 0.045 mg/mg, 0.085 mg/mg |
| Benzoyl peroxide | Solution: 7 wt. % |
| Benzoyl peroxide | Gel: 0.05 mg/mg, 0.1 mg/mg |
| Benzoyl peroxide | Foam: 55 mg/mL, 95 mg/mL |
| Benzoyl peroxide | Cream: 5.5 wt. % |
| | Wash solution: 7 wt. % |
| Benzoyl peroxide | Gel: 0.05 mg/mg |
| Benzoyl peroxide | Gel: 0.03 mg/mg |
| Benzoyl peroxide | Cream: 100 mg/mL |
| Benzoyl peroxide | Wash solution: 7 wt. % |
| Salicylic acid | Medicated liquid soap: 20 mg/mL |
| Salicylic acid | Cream: 60 mg/mL |
| | Lotion: 60 mg/mL |
| Salicylic acid | Gel: 0.02 mg/mg |
| | Lotion: 5 mg/mL |
| | Medicated liquid soap: 5 mg/mL |
| Salicylic acid | Liquid: 0.5 wt. % |
| Salicylic acid | Gel: 0.17 mg/mg |
| | Solution: 170 mg/mL |
| | Medicated patch: 0.4 mg/mg |
| Salicylic acid | Lotion: 20 mg/mL |
| Salicylic acid | Medicated shampoo: 30 mg/mL |
| Salicylic acid | Solution: 260 mg/mL |
| Salicylic acid | Gel: 0.05 mg/mg, 0.06 mg/mg, 0.17 mg/mg |
| Salicylic acid | Gel: 0.02 mg/mg |
| Salicylic acid | Solution: 170 mg/mL |
| Salicylic acid | Medicated shampoo: 2 wt. % |
| Salicylic acid | Solution: 20 mg/mL |
| | Medicated Pad: 20 mg/mL |
| | Medicated liquid soap: 20 mg/mL |
| Salicylic acid | Gel: 0.17 mg/mg |
| Salicylic acid | Solution: 20 mg/mL |
| | Foam: 20 mg/mL |
| | Medicated bar soap: 0.02 mg/mg |
| Salicylic acid | Solution: 170 mg/mL |
| Salicylic acid | Lotion: 60 mg/mL |
| | Cream: 60 mg/mL |
| Salicylic acid | Lotion: 60 mg/mL |
| | Cream: 60 mg/mL |
| | Medicated shampoo: 60 mg/mL |
| Salicylic acid | Lotion: 60 mg/mL |
| | Cream: 60 mg/mL |
| Salicylic acid | Foam: 60 mg/mL |
| Salicylic acid | Foam: 60 mg/mL |
| Salicylic acid | Solution: 30 mg/mL |
| Salicylic acid | Medicated bar soap: 0.01 mg/mg |
| | Medicated pad: 5 mg/mL, 20 mg/mL |
| | Gel: 0.02 mg/mg |
| Salicylic acid | Medicated shampoo: 30 mg/mL |
| Salicylic acid | Medicated pad: 10 mg/mL, 20 mg/mL |
| Salicylic acid/Sulfur | Medicated bar soap: 0.02 mg/mg SA/ |
| | 0.02 mg/mg sulfur |
| Sulfur | Lotion: 50 mg/mL |
| Sulfur | Medicated bar soap: 0.03 mg/mg |
| | Lotion: 40 mg/mL |
| Sulfur | Medicated shampoo: 20 mg/mL |
| Sulfur | Cream: 64 mg/mL |
| Sulfur | Cream: 50 mg/mL |
| Sulfur | Medicated bar soap: 0.05 mg/mg |
| Resorcinol | Lotion: 30 mg/mL |
| Resorcinol | Ointment: 0.02 mg/mg |
| Adapalene | Medicated pad: 1 mg/mL |
| | gel: 0.003 mg/mg, 0.001 mg/mg |
| | Cream: 1 mg/mL |
| | Lotion: 1 mg/mL |
| | Solution: 1 mg/mL |

TABLE 3

| ACTIVE | AMOUNT |
|---|---|
| alcloxa | 0.05 wt. %, 0.1 wt. %, 0.13 wt. %, 0.16 wt. % |
| alkyl isoquinolinium bromide | 0.1 wt. %, 0.5 wt. % solution |
| aluminum chlorohydrex | 10 wt. % |
| aluminum hydroxide | 0.275 wt. %, 1.2 wt. %, 2 wt. %, 320 mg/5 mL, |
| | 800 mg/10 mL, 400 mg/5 mL, 200 mg/5 mL, |
| | 400 mg/10 mL, 1200 mg/30 mL |
| benzocaine | 2, 5, 6, 10, 14, 16, 20 wt. %, 20 mg/g |
| benzoic acid | 12x in 1 mL (oral), 30x, 60x, 200x (oral) |
| benzoyl peroxide | 2.5-10 wt. % |
| boric acid | 12x in 1 mL (oral), 30x in 1 mL (oral), 60x, |
| | 200x |
| calcium polysulfide | 25-35 wt. % |
| calcium thiosulfate | |
| camphor | 4x, 3x, 12x, 0.2 wt. %, 0.5 wt. %, 0.7 wt. %, |
| | 1 wt. %, 2 wt. %, 3 wt. %, 3.1 wt. %, 5 wt. %, |
| | 3.18 wt. % (synthetic), 6.2 wt. % (synthetic), |
| | 7.79 wt. % |
| chlorhydroxyquinoline (cloxyquin) | |
| chloroxylenol | 0.1 wt. % (w/w), 3 mg/1 mL, 0.24 wt. %, 0.25 |
| | wt. %, 0.3 wt. %, 0.4 wt. %, 0.45 wt. %, 0.5 |
| | wt. %, 0.6 wt. %, 0.8 wt. %, 1 wt. % (hand |
| | wash), 1.75 wt. % (hand wash) |
| coal tar | 0.5 wt. %, 0.8 wt. %, 1 wt. %, 1.25 wt. %, 1.5 |
| | wt. %, 1.7 wt. %, 2 wt. %, 2.3 wt. %, 5 wt. % |
| dibenzothiophene | 5 wt. % |
| estrone | 6x in 1 mL (oral), 8x in 1 mL (oral), 12x in |
| | 1 mL (oral), 30x in 1 mL (oral), 200C in 1 mL, |
| magnesium aluminum silicate | 0.1-5 wt. % |
| magnesium sulfate | 1X/fl oz, 5X/fl oz, 6X, 9X, 4X in 1 mL, 12X |
| | in 1.2 oz, 4 wt. %, 30C in 1 mL, |
| phenol | 12X in 1 mL, 30X in 1 mL, 60X, 200X |
| phenolate sodium | |
| phenyl salicylate | 18X in 59.1 mL |
| povidone-iodine | 2 wt. %, 5 wt. %, 10 wt. %, |
| pyrilamine maleate | 15 mg/caplet; 12.5 mg/5 mL; 50 mg/tablet; |
| | 8.33 mg/5 mL; 30 mg/tablet; 7.5 mg/5 mL; |
| | 16 mg/tsp; 12.5 mL/15 mL |
| resorcinol (as single ingredient only) | 2-3 wt. %; 30X in 1 mL |
| resorcinol (when combined with sulfur) | 2 wt. % |
| resorcinol monoacetate (as single | 3-8 wt. % |
| ingredient only) | |
| resorcinol monoacetate (when combined | 3 wt. % |
| with sulfur) | |
| salicylic acid | 0.5-2 wt. % or 2-5 wt. % |
| sodium borate | 30X/1 g; 4X, 12X, 3X, 1X in 240 mL; 6X; |
| | 30C/1 mL; 9X; 12X in 30 mL; 8X in 59.1 mL; |
| | 7X; 5C in 25 mL; |
| sodium thiosulfate | 8 wt. %, 125, 25 wt. % |
| sulfur | 3-10 wt. % |
| sulfur (when combined with resorcinol, | 3-8 wt. % |
| resorcinol monoacetate) | |
| tetracaine hydrochloride | 2 wt. %, 4 wt. % |
| thymol | 0.063 wt. %, 0.064 wt. %, |
| vitamin E | 13,650 IU/30 mL (oil); 11 IU (capsule) |
| zinc oxide | 0.015, 3.5 wt. %, 5 wt. %, 10 wt. %, 12 wt. %, |
| | 15 wt. %, 16 wt. %, 24.6 wt. %, 25 wt. %, |
| | 27.81 wt. %, 40 wt. % |
| zinc stearate | |
| zinc sulfide | |
| Glycolic acid | 10 wt. %, 7 wt. %, 4 wt. % |
| Vitamin A | Solution: 2500 IU; 10,000 IU |

### Enumerated Embodiments

Specific enumerated embodiments <1> to <101> provided below are for illustration purposes only, and do not otherwise limit the scope of the disclosed subject matter, as defined by the claims. These enumerated embodiments encompass all combinations, sub-combinations, and multiply referenced (e.g., multiply dependent) combinations described therein.
<1> A topical anti-acne composition including the formulation of any one of Production Examples 1-307, further including a cannabinoid, terpene, flavonoid, or combination thereof.
<2> A topical anti-acne composition including the formulation of any one of Production Examples 1-307, further including a cannabinoid.
<3> A topical anti-acne composition including the formulation of any one of Production Examples 1-307, further including a cannabinoid obtained as a distillate from Cannabis.
<4> A topical anti-acne composition including the formulation of any one of Production Examples 1-307, further including a cannabinoid obtained as an extract from Cannabis.
<5> A topical anti-acne composition including the formulation of any one of Production Examples 1-307, further including a cannabinoid obtained as a resin from Cannabis.
<6> A topical anti-acne composition including the formulation of any one of Production Examples 1-307, further including a cannabinoid isolate obtained from Cannabis.
<7> A topical anti-acne composition including the formulation of any one of Production Examples 1-307, further including a cannabinoid obtained from Cannabis indica, Cannabis ruderalis, or Cannabis sativa.
<8> A topical anti-acne composition including the formulation of any one of Production Examples 1-307, further including a cannabinoid present as an oil from Cannabis.
<9> A topical anti-acne composition including the formulation of any one of Production Examples 1-307, further including a cannabinoid present as hempseed oil.
<10> A topical anti-acne composition including the formulation of any one of Production Examples 1-307, further including a cannabinoid that is synthetically prepared.
<11> A topical anti-acne composition including the formulation of any one of Production Examples 1-307, further including a cannabinoid that is at least one of THC (tetrahydrocannabinol), THCA (tetrahydrocannabinolic acid), CBD (cannabidiol), CBDA (cannabidiolic acid), CBN (cannabinol), CBG (cannabigerol), CBC (cannabichromene), CBL (cannabicyclol) CBV (cannabivarin), THCV (tetrahydrocannabivarin), CBDV (cannabidivarin), CBCV (cannabichromevarin), CBGV (cannabigerovarin), CBGM (cannabigerol monomethyl ether), CBE (cannabielsoin), and CBT (cannabicitran).
<12> A topical anti-acne composition including the formulation of any one of Production Examples 1-307, further including a cannabinoid that is at least one of CBD and THC.
<13> A topical anti-acne composition including the formulation of any one of Production Examples 1-307, further including a terpene.
<14> A topical anti-acne composition including the formulation of any one of Production Examples 1-307, further including a terpene that is a sesquiterpene.
<15> A topical anti-acne composition including the formulation of any one of Production Examples 1-307, further including a terpene obtained as a distillate from plant matter.
<16> A topical anti-acne composition including the formulation of any one of Production Examples 1-307, further including a terpene obtained as an extract from plant matter.
<17> A topical anti-acne composition including the formulation of any one of Production Examples 1-307, further including a terpene obtained as a resin from plant matter.
<18> A topical anti-acne composition including the formulation of any one of Production Examples 1-307, further including a terpene obtained from Cannabis sativa, Syzygium aromaticum (cloves), rosemary, or hops.
<19> A topical anti-acne composition including the formulation of any one of Production Examples 1-307, further including a terpene that is synthetically prepared.
<20> A topical anti-acne composition including the formulation of any one of Production Examples 1-307, further including a terpene that is Beta-Caryophyllene.
<21> The topical anti-acne composition of any one of the above embodiments, wherein the amount of external anti-acne agent expressed in terms of concentration (weight percent), present in the formulation of the any one of Production Examples 1-307, remains unchanged with the addition of the cannabinoid, terpene, flavonoid, or combination thereof. For Example, if the amount of external anti-acne agent present in the formulation of the any one of Production Examples 1-307 is 1 wt. % salicylic acid. With the addition of the cannabinoid, terpene, flavonoid, or combination thereof, the resulting amount of the salicylic acid will remain at 1 wt. %.
<22> The topical anti-acne composition of any one of the above embodiments, wherein the amount of inactive ingredient expressed in terms of concentration (weight percent), present in the formulation of the any one of Production Examples 1-307, decreases with the amount added of the cannabinoid, terpene, flavonoid, or combination thereof. For Example, if the amount of external anti-acne agent present in the formulation of the any one of Production Examples 1-307 is 1 wt. % salicylic acid, with the addition of the cannabinoid, terpene, flavonoid, or combination thereof, the resulting amount of the salicylic acid will be less than 1 wt. %.
<23> The topical anti-acne composition of any one of the above embodiments, wherein the aggregate amount of inactive ingredients, present in the formulation of the any one of Production Examples 1-307, decreases in proportion to the amount added of the cannabinoid, terpene, flavonoid, or combination thereof. For Example, if the aggregate amount of inactive ingredients present in the formulation of the any one of Production Examples 1-307 is 90 wt. %. With the addition of 1 wt. % of cannabinoid, terpene, flavonoid, or combination thereof, the resulting aggregate amount of those inactive ingredients will be 89 wt. %.
<24> The topical anti-acne composition of any one of the above embodiments, wherein the aggregate amount of active ingredient (external anti-acne agent) and inactive ingredient in terms of concentration (weight percent), present in the formulation of the any one of Production Examples 1-307, decreases in proportion to the amount added of the cannabinoid, terpene, flavonoid, or combination thereof. For Example, the aggregate amount of active and inactive ingredients present in the formulation of the any one of Production Examples 1-307 is 100 wt. %. With the addition of 1 wt. % of cannabinoid, terpene, flavonoid, or combination thereof, the resulting aggregate amount of those active and inactive ingredients will be 99 wt. %.
<25> The topical anti-acne composition of any one of the above embodiments, wherein the cannabinoid, terpene, flavonoid, or combination thereof, is present in an aggregate amount of up to 20 wt. %.
<26> The topical anti-acne composition of any one of the above embodiments, wherein the cannabinoid, terpene, flavonoid, or combination thereof, is present in an aggregate amount of up to 10 wt. %.
<27> The topical anti-acne composition of any one of the above embodiments, wherein the cannabinoid, terpene, flavonoid, or combination thereof, is present in an aggregate amount of up to 5 wt. %.
<28> The topical anti-acne composition of any one of the above embodiments, wherein the cannabinoid, terpene, flavonoid, or combination thereof, is present in an aggregate amount of up to 1 wt. %.
<29> The topical anti-acne composition of any one of the above embodiments, wherein the cannabinoid, terpene, flavonoid, or combination thereof, is present in an aggregate amount of up to 0.5 wt. %.
<30> The topical anti-acne composition of any one of the above embodiments, wherein the cannabinoid, terpene, flavonoid, or combination thereof, is present in an aggregate amount of 0.330±0.1 wt. %.
<31> The topical anti-acne composition of any one of the above embodiments, wherein the cannabinoid, terpene, flavonoid, or combination thereof, is present in an aggregate amount of 0.01 to 20 wt. %.
<32> The topical anti-acne composition of any one of the above embodiments, wherein the cannabinoid, terpene, flavonoid, or combination thereof, is present in an aggregate amount of 0.01 to 10 wt. %.
<33> The topical anti-acne composition of any one of the above embodiments, wherein the cannabinoid, terpene, flavonoid, or combination thereof, is present in an aggregate amount of 0.01 to 5 wt. %.
<34> The topical anti-acne composition of any one of the above embodiments, wherein the cannabinoid, terpene, flavonoid, or combination thereof, is present in an aggregate amount of 0.01 to 2.5 wt. %.
<35> The topical anti-acne composition of any one of the above embodiments, wherein the cannabinoid, terpene, flavonoid, or combination thereof, is present in an aggregate amount of 0.01 to 1 wt. %.
<36> The topical anti-acne composition of any one of the above embodiments, wherein the cannabinoid, terpene, flavonoid, or combination thereof, is present in an aggregate amount of 0.01 to 0.5 wt. %.
<37> The topical anti-acne composition of any one of the above embodiments, wherein the cannabinoid, terpene, flavonoid, or combination thereof, is present in an aggregate amount of 0.1 to 20 wt. %.
<38> The topical anti-acne composition of any one of the above embodiments, wherein the cannabinoid, terpene, flavonoid, or combination thereof, is present in an aggregate amount of 0.1 to 10 wt. %.
<39> The topical anti-acne composition of any one of the above embodiments, wherein the cannabinoid, terpene, flavonoid, or combination thereof, is present in an aggregate amount of 0.1 to 5 wt. %.
<40> The topical anti-acne composition of any one of the above embodiments, wherein the cannabinoid, terpene, flavonoid, or combination thereof, is present in an aggregate amount of 0.05 to 2.5 wt. %.
<41> The topical anti-acne composition of any one of the above embodiments, wherein the cannabinoid, terpene, flavonoid, or combination thereof, is present in an aggregate amount of 0.05 to 1 wt. %.
<42> The topical anti-acne composition of any one of the above embodiments, wherein the cannabinoid, terpene, flavonoid, or combination thereof, is present in an aggregate amount of 0.05 to 0.5 wt. %.
<43> The topical anti-acne composition of any one of the above embodiments, which is in the form of a gel, pump gel, gel packet, cream, lotion, roll-on liquid, roll-on gel, spray, pump spray, aerosol spray, stick, patch, ointment, liniment, or balm.
<44> A topical anti-acne composition including the formulation of any one of Production Examples 1-307, further including a cannabinoid, terpene, flavonoid, or combination thereof; wherein the cannabinoid, terpene, flavonoid, or combination thereof, while present in the aggregate amounts, are characterized as inactive ingredients.
<45> A topical anti-acne composition including the formulation of any one of Production Examples 1-307, further including a cannabinoid, terpene, flavonoid, or combination thereof; wherein the cannabinoid, terpene, flavonoid, or combination thereof, while present in the aggregate amounts, are characterized as an excipient.
<46> A topical anti-acne composition including the formulation of any one of Production Examples 1-307, further including a cannabinoid, terpene, flavonoid, or combination thereof; wherein the cannabinoid, terpene, flavonoid, or combination thereof, while present in the aggregate amounts, are characterized as active ingredients.
<47> A topical anti-acne composition including the formulation of any one of Production Examples 1-307, further including a cannabinoid, terpene, flavonoid, or combination thereof; wherein the cannabinoid, terpene, flavonoid, or combination thereof, while present in the aggregate amounts, are characterized as an external anti-acne agent.
<48> A topical anti-acne composition including one or more external anti-acne agents of Production Example 74, further including a cannabinoid, terpene, flavonoid, or combination thereof, and a carrier system.
<49> A topical anti-acne composition including one or more external anti-acne agents of Production Example 74, further including a cannabinoid, and a carrier system.
<50> A topical anti-acne composition including one or more external anti-acne agents of Production Example 74, further including a cannabinoid obtained as a distillate from Cannabis, and a carrier system.
<51> A topical anti-acne composition including one or more external anti-acne agents of Production Example 74, further including a cannabinoid obtained as an extract from Cannabis, and a carrier system.
<52> A topical anti-acne composition including one or more external anti-acne agents of Production Example 74, further including a cannabinoid obtained as a resin from Cannabis, and a carrier system.
<53> A topical anti-acne composition including one or more external anti-acne agents of Production Example 74, further including a cannabinoid isolate obtained from Cannabis, and a carrier system.
<54> A topical anti-acne composition including one or more external anti-acne agents of Production Example 74, further including a cannabinoid obtained from Cannabis indica, Cannabis ruderalis, or Cannabis sativa, and a carrier system.
<55> A topical anti-acne composition including one or more external anti-acne agents of Production Example 74, further including a cannabinoid present as an oil from Cannabis, and a carrier system.
<56> A topical anti-acne composition including one or more external anti-acne agents of Production Example 74, further including a cannabinoid present as hempseed oil, and a carrier system.
<57> A topical anti-acne composition including one or more external anti-acne agents of Production Example 74, further including a cannabinoid that is synthetically prepared, and a carrier system.
<58> A topical anti-acne composition including one or more external anti-acne agents of Production Example 74, further including a cannabinoid that is at least one of THC (tetrahydrocannabinol), THCA (tetrahydrocannabinolic acid), CBD (cannabidiol), CBDA (cannabidiolic acid), CBN (cannabinol), CBG (cannabigerol), CBC (cannabichromene), CBL (cannabicyclol) CBV (cannabivarin), THCV (tetrahydrocannabivarin), CBDV (cannabidivarin), CBCV (cannabichromevarin), CBGV (cannabigerovarin), CBGM (cannabigerol monomethyl ether), CBE (cannabielsoin), and CBT (cannabicitran), and a carrier system.
<59> A topical anti-acne composition including one or more external anti-acne agents of Production Example 74, further including a cannabinoid that is at leak one of CBD and THC, and a carrier system.
<60> A topical anti-acne composition including one or more external anti-acne agents of Production Example 74, further including a terpene, and a carrier system.
<61> A topical anti-acne composition including one or more external anti-acne agents of Production Example 74, further including a terpene that is a sesquiterpene, and a carrier system.
<62> A topical anti-acne composition including one or more external anti-acne agents of Production Example 74, further including a terpene obtained as a distillate from plant matter, and a carrier system.
<63> A topical anti-acne composition including one or more external anti-acne agents of Production Example 74, further including a terpene obtained as an extract from plant matter, and a carrier system.
<64> A topical anti-acne composition including one or more external anti-acne agents of Production Example 74, further including a terpene obtained as a resin from plant matter, and a carrier system.
<65> A topical anti-acne composition including one or more external anti-acne agents of Production Example 74, further including a terpene obtained from Cannabis sativa, Syzygium aromaticum (cloves), rosemary, or hops, and a carrier system.
<66> A topical anti-acne composition including one or more external anti-acne agents of Production Example 74, further including a terpene that is synthetically prepared, and a carrier system.
<67> A topical anti-acne composition including one or more external anti-acne agents of Production Example 74, further including a terpene that is Beta-Caryophyllene, and a carrier system.
<68> The topical anti-acne composition of any one of the above embodiments, wherein the cannabinoid, terpene, flavonoid, or combination thereof, is present in an aggregate amount of up to 20 wt. %.
<69> The topical anti-acne composition of any one of the above embodiments, wherein the cannabinoid, terpene, flavonoid, or combination thereof, is present in an aggregate amount of up to 10 wt. %.
<70> The topical anti-acne composition of any one of the above embodiments, wherein the cannabinoid, terpene, flavonoid, or combination thereof, is present in an aggregate amount of up to 5 wt. %.
<71> The topical anti-acne composition of any one of the above embodiments, wherein the cannabinoid, terpene, flavonoid, or combination thereof, is present in an aggregate amount of up to 1 wt. %.
<72> The topical anti-acne composition of any one of the above embodiments, wherein the cannabinoid, terpene, flavonoid, or combination thereof, is present in an aggregate amount of up to 0.5 wt. %.
<73> The topical anti-acne composition of any one of the above embodiments, wherein the cannabinoid, terpene, flavonoid, or combination thereof, is present in an aggregate amount of 0.330±0.1 wt. %.
<74> The topical anti-acne composition of any one of the above embodiments, wherein the cannabinoid, terpene, flavonoid, or combination thereof, is present in an aggregate amount of 0.01 to 20 wt. %.
<75> The topical anti-acne composition of any one of the above embodiments, wherein the cannabinoid, terpene, flavonoid, or combination thereof, is present in an aggregate amount of 0.01 to 10 wt. %.
<76> The topical anti-acne composition of any one of the above embodiments, wherein the cannabinoid, terpene, flavonoid, or combination thereof, is present in an aggregate amount of 0.01 to 5 wt. %.
<77> The topical anti-acne composition of any one of the above embodiments, wherein the cannabinoid, terpene, flavonoid, or combination thereof, is present in an aggregate amount of 0.01 to 2.5 wt. %.
<78> The topical anti-acne composition of any one of the above embodiments, wherein the cannabinoid, terpene, flavonoid, or combination thereof, is present in an aggregate amount of 0.01 to 1 wt. %.
<79> The topical anti-acne composition of any one of the above embodiments, wherein the cannabinoid, terpene, flavonoid, or combination thereof, is present in an aggregate amount of 0.01 to 0.5 wt. %.
<80> The topical anti-acne composition of any one of the above embodiments, wherein the cannabinoid, terpene, flavonoid, or combination thereof, is present in an aggregate amount of 0.1 to 20 wt. %.
<81> The topical anti-acne composition of any one of the above embodiments, wherein the cannabinoid, terpene, flavonoid, or combination thereof, is present in an aggregate amount of 0.1 to 10 wt. %.
<82> The topical anti-acne composition of any one of the above embodiments, wherein the cannabinoid, terpene, flavonoid, or combination thereof, is present in an aggregate amount of 0.1 to 5 wt. %.
<83> The topical anti-acne composition of any one of the above embodiments, wherein the cannabinoid, terpene, flavonoid, or combination thereof, is present in an aggregate amount of 0.05 to 2.5 wt. %.
<84> The topical anti-acne composition of any one of the above embodiments, wherein the cannabinoid, terpene, flavonoid, or combination thereof, is present in an aggregate amount of 0.05 to 1 wt. %.
<85> The topical anti-acne composition of any one of the above embodiments, wherein the cannabinoid, terpene, flavonoid, or combination thereof, is present in an aggregate amount of 0.05 to 0.5 wt. %.
<86> The topical anti-acne composition of any one of the above embodiments, which is in the form of a gel, pump gel, gel packet, cream, lotion, roll-on liquid, roll-on gel, spray, pump spray, aerosol spray, stick, patch, ointment, liniment, or balm.
<87> A topical anti-acne composition including one or more external anti-acne agents of Production Example 74, further including a cannabinoid, terpene, flavonoid, or combination thereof, and a carrier system; wherein the cannabinoid, terpene, flavonoid, or combination thereof, while present in the aggregate amounts, are characterized as inactive ingredients.
<88> A topical anti-acne composition including one or more external anti-acne agents of Production Example 74, further including a cannabinoid, terpene, flavonoid, or combination thereof, and a carrier system; wherein the cannabinoid, terpene, flavonoid, or combination thereof, while present in the aggregate amounts, are characterized as an excipient.
<89> A topical anti-acne composition including one or more external anti-acne agents of Production Example 74, further including a cannabinoid, terpene, flavonoid, or combination thereof, and a carrier system; wherein the cannabinoid, terpene, flavonoid, or combination thereof, while present in the aggregate amounts, are characterized as an active ingredient.
<90> A topical anti-acne composition including one or more external anti-acne agents of Production Example 74, further including a cannabinoid, terpene, flavonoid, or combination thereof, and a carrier system; wherein the cannabinoid, terpene, flavonoid, or combination thereof, while present in the aggregate amounts, are characterized as an external anti-acne agent.
<91> The topical anti-acne composition of any one of the above embodiments, which is in the form of a cream, gel, lotion, patch, liniment, spray, roll-on, stick, or balm.
<92> The topical anti-acne composition of any one of the above embodiments, having a total THC content of less than 0.05 mg/mL, wherein the total THC content is defined as the amount of THCA/mL*0.877, plus the amount of THC/mL*1, expressed as:

Total THC content=(amount of THCA/mL*0.877)+(amount of THC/mL*1)<0.05 mg/mL
<93> The topical anti-acne composition of any one of the above embodiments, having a total THC content of less than 0.025 mg/mL, wherein the total THC content is defined as the amount of THCA/mL*0.877, plus the amount of THC/mL*1, expressed as:

Total THC content=(amount of THCA/mL*0.877)+(amount of THC/mL*1)<0.025 mg/mL
<94> The topical anti-acne composition of any one of the above embodiments, substantially free from (a)-(e):

(a) Tetrahydrocannabinol (THC),

(b) Tetrahydrocannabinolic Acid (THCA),

(c) Cannabidiolic Acid (CBDA),

(d) Cannabinol (CBN),

(e) Cannabigerol (CBG),

such that any of (a)-(e) present in the topical anti-acne composition is present such that the topical cream includes each in no more than 0.01 mg/mL.
<95> The topical anti-acne composition of any one of the above embodiments, substantially free from (a)-(e):

(a) Tetrahydrocannabinol (THC),

(b) Tetrahydrocannabinolic Acid (THCA),

(c) Cannabidiolic Acid (CBDA),

(d) Cannabinol (CBN),

(e) Cannabigerol (CBG),

such any one or more of (a)-(e) that is present in the topical anti-acne composition is present in a total amount of up to 0.02 mg/mL.
<96> A method that includes topically administering to a subject the topical anti-acne composition of any one of the above embodiments.
<97> The method of embodiment <96>, wherein the topical composition is administering to the skin surface of the subject to treat, prevent, or manage at least one of: (i) acne, (ii) inflammatory acne, (iii) acne causing bacteria (e.g., acne vulgaris), (iv) rosacea, (v) inflammation, (vi) clogged pores, (vii) dry sebum, (viii) inflammatory lesions (blemishes), (ix) pimples, (x) blackheads, and (xi) whiteheads.
<98> The method of embodiment <96>, which effectively: prevents and kills acne bacteria, reduces the number and/or severity of acne pimples, clears acne blemishes, helps prevent development of new acne blemishes, cleanses the skin, removes excess oil, reduces the size and redness of acne breakout, renews skin, normalizes skin, exfoliates the skin, provides smooth & soft skin texture, invigorates skin by lifting away dead skin cells, clears skin, reduces appearance of pores, helps balance skin's pH level, provides clearer complexion, and/or provides softer & smoother skin.
<99> The method of any one of embodiments <96> to <98>, wherein the external anti-acne agent is applied to a clean and dry topical skin surface of the subject.
<100> The method of any one of embodiments <96> to <99>, wherein the subject is a human.
<101> The method of any one of embodiments <96> to <100>, wherein the subject is a human of at least 12 years old.

1. A method comprising topically administering to a skin surface of a human subject afflicted with acne or at risk thereof, the topical anti-acne composition comprising:
external anti-acne agent;
solvent;
emulsifier;
thickening agent;
emollient; and
at least one of a cannabinoid, terpene, and flavonoid;
wherein,
the topical anti-acne composition is substantially free from (a)-(c): (a) Cannabidiolic Acid (CBDA), (b) Cannabinol (CBN), and (c) Cannabigerol (CBG); such that any of (a)-(c) present in the topical anti-acne composition is present such that the topical anti-acne composition includes each in no more than 0.01 mg/mL; and
the topical anti-acne composition has a total THC content of less than 0.02 mg/mL, wherein the total THC content is defined as the amount of THCA*0.877, plus the amount of THC*1, expressed as:
Total THC content=(amount of THCA/mL*0.877)+(amount of THC/mL*1)<0.02 mg/mL.

2. The method of claim 1, wherein the topical anti-acne composition is administered to the skin surface of the subject to treat, prevent, ameliorate, or manage at least one of: (i) acne, (ii) inflammatory acne, (iii) acne causing bacteria (e.g., acne vulgaris), (iv) rosacea, (v) inflammation, (vi) clogged pores, (vii) dry sebum, (viii) inflammatory lesions (blemishes), (ix) pimples, (x) blackheads, and (xi) whiteheads.

3. The method of 1, which effectively: prevents & kills acne bacteria, reduces the number and/or severity of acne pimples, clears acne blemishes, helps prevent development of new acne blemishes, cleanses the skin, removes excess oil, reduces the size and redness of acne breakout, renews skin, normalizes skin, exfoliates the skin, provides smooth & soft skin texture, invigorates skin by lifting away dead skin cells, clears skin, reduces appearance of pores, helps balance skin's pH level, provides clearer complexion, and/or provides softer & smoother skin.

4. The method of 1, wherein the external anti-acne agent comprises at least one of adapalene, alcloxa, alkyl isoquinolinium bromide, aluminum chlorohydrex, aluminum hydroxide, azelaic acid, benzocaine, benzoic acid, benzoyl peroxide, boric acid, calcium polysulfide, calcium thiosulfate, camphor, cephalexin, chlorhydroxyquinoline (cloxyquin), chloroxylenol, cloxyquin (chlorhydroxyquinoline), clindamycin, coal tar, dapsone, dibenzothiophene, doxycycline, erythromycin, estrone, glycolic acid, hydrocortisone, isotretinoin, magnesium aluminum silicate, magnesium sulfate, metronidazole, minocycline, phenol, phenolate sodium, phenyl salicylate, povidone-iodine, pyrilamine maleate, resorcinol, resorcinol monoacetate, Retin-A, salicylic acid, sodium borate, sodium thiosulfate, spironolactone, sulfur, tazarotene, tetracaine hydrochloride, thymol, tretinoin, vitamin A, vitamin E, zinc oxide, zinc stearate, and zinc sulfide.

5. The method of 1, wherein the external anti-acne agent is present in an aggregate amount of up to 20 wt. %.

6. The method of 1, wherein the solvent comprises at least one of water, glycerin, propylene glycol, propanediol, butylene glycol, ethoxydiglycol, isododecane, isohexadecane, octyldodecanol, hexylene glycol, 1,2-hexane diol, and dicaprylyl carbonate.

7. The method of 1, wherein the solvent is present in 60-80 wt. %.

8. The method of 1, wherein the emulsifier comprises at least one of polysorbate 60, laureth-4, potassium cetyl sulfate, cetyl alcohol, cetearyl alcohol, stearyl alcohol, glyceryl stearate, propylene glycol, polyglyceryl-6 laurate, ceteareth-20, PEG-100 stearate, sodium lauroyl lactylate, myristyl myristate, carbomer, polysorbate 80, polawax, sorbitan stearate, gum Arabic, brassica alcohol, carbomer 980 QD, sodium stearate, polyhydroxystearic acid, PEG-150 distearate, glyceryl oleate, emulsifying wax, glyceryl monooleate, phospholipids, polyoxyethylene alkyl ethers, polyoxyethylene, castor oil derivatives, sorbitan fatty acid esters, polyoxyethylene stearates, polyoxylglycerides, polysorbate, and sorbitan esters.

9. The method of 1, wherein the emulsifier is present in 3-8 wt. %.

10. The method of 1, wherein the thickening agent comprises at least one of Carbopol 980, carbomer, cetyl alcohol, stearic acid, carnauba wax, hydroxyethyl cellulose, guar gum, xanthan gum, gelatin, magnesium aluminum silicate, silica, bentonite, cetyl palmitate, ammonium acryloyldimethyltaurate, cetearyl alcohol, glucose-D, hectorite gel, stearyl palmitate, gum arabic, hydroxypropyl starch phosphate, tapioca starch, acrylates octylacrylamide copolymer, carbomer 940, polyamide 3, castor wax, hydroxypropyl methylcellulose, caesalpinia spinosa gum, brassica alcohol, carbomer 980 QD, sodium stearate, polyhydroxystearic acid, tribehenin, arrowroot starch, rice starch, candelilla wax, beeswax, ozokerite wax, sunflower wax, PEG-150 distearate, polyacrylate crosspolymer-6, acrylates C10-30 alkyl acrylate crosspolymer, hydroxypropyl guar, and cyclopentasiloxane (silicone gel).

11. The method of 1, wherein the thickening agent is present in 0.3-3 wt. %.

12. The method of 1, wherein the emollient comprises at least one of beeswax, olive oil, coconut oil, lanolin oil (isolanolin), petrolatum (petroleum jelly), mineral oil, glycerin, methyl glyceth, zinc oxide, goat milk, butyl stearate, diglycol laurate, C12-15 alkyl benzoate, aloe vera palmitate, butylene glycol, ethylhexyl palmitate, isododecane, isohexadecane, isopropyl myristate, octyldodecanol, PEG-40 hydrogenated castor oil, pentylene glycol BIO, (1,3) propanediol, allantoin, lecithin, caprylic capric triglyceride, octyl deodecanol, cetearyl alcohol, squalene, squalene, tocopheryl acetate, polyglyceryl-6 laurate, ceteareth-20, dimethicone, PEG-100 stearate, octyldodecanol, caprylyl glycol, dicaprylyl carbonate, myristyl myristate, hectorite gel, stearyl palmitate, polyamide 3, sorbitol, polyhydroxystearic acid, lauryl laurate, and cyclopentasiloxane (silicone gel).

13. The method of 1, wherein the emollient is present in 10-22 wt. % of the topical anti-acne composition.

14. The method of 1, wherein the topical anti-acne composition is formulated as a cream, lotion, ointment, or gel.

15. The method of 1, wherein the topical anti-acne composition further comprises at least one of a pH adjusting agent, antioxidant, buffer, transdermal delivery agent, preservative, fragrance, coloring agent, and exfoliant.

16. The method of 1, wherein the topical anti-acne composition further comprises a pH adjusting agent comprising at least one of triethanolamine, citric acid, sodium gluconate, magnesium hydroxide, sodium bicarbonate, and lactic acid.

17. The method of 1, wherein the topical anti-acne composition further comprises a preservative comprising at least one of butylparaben, ethylparaben, methylparaben, propylparaben, sorbic acid, benzyl alcohol, salicylic acid, formaldehyde, tetrasodium ethylenediaminetetra-acetic acid (EDTA), neroli hydrosol, phenoxyethanol, ethylhexylglycerin, methylisothiazolinone, methylchloroisothiazolinone, citric acid, and tetrasodium glutamate diacetate.

18. The method of 1, wherein the topical anti-acne composition further comprises a transdermal delivery agent comprising at least one of dimethyl sulfoxide (DMSO), transcutol® (2-(2-ethoxyethoxy) ethanol), ethanol, and isopropyl alcohol.

19. The method of 1, wherein the topical anti-acne composition further comprises an antioxidant comprising at least one of tocopherol (vitamin E), tocopheryl acetate (vitamin E acetate), anthocyanins, proanthocyanins, alpha-lipoic acid, catechins, retinol (vitamin A), beta-carotene (vitamin A), Coenzyme Q10, chlorogenic acid, curcumin, vitamin C, theograndin, luteolin, acetophenone derivatives, monoterpenes, sesquiterpenes, triterpenes, phytosterols, ferulic acid, and lycopene.

20. The method of 1, wherein the topical anti-acne composition further comprises an antioxidant present in 0.05-0.5 wt. %.

21. The method of 1, wherein the topical anti-acne composition further comprises a fragrance comprising at least one of blood orange, citrus punch, clean linen, coconut, coconut lime verbena, coral reef, lemon verbena, mandarin berry, mangosteen, natural rose, neroli, pineapple lily, pink grapefruit and passion fruit, white peach and ginger, white tea, lavender, peppermint, sweet fennel, sweet orange, tea tree, woodland essential, vanilla, cherry blossom, white jasmine, apple blossom, pumpkin, cinnamon, peony, apple, blackberry, cranberry, plum blossom, grapefruit, ginger flower, freesia petal, chamomile, eucalyptus, violet, sweet pea, frangipani, pomegranate, honeydew, cantaloupe, white gardenia, pink nectarine, cotton musk, magnolia blossom, sage, cedarwood, freesia, black currant, white patchouli, sweet almond, and cashmere musk.

22. The method of 1, wherein the topical anti-acne composition further comprises a coloring agent comprising at least one of caramel colorant, Red No. 6 D&C Lake liquid, Red No. 7 D&C Lake liquid, Blue No. 1 FD&C Lake, Red No. 28 D&C Lake, Red No. 40 FD&C Lake, Red No. 7 D&C Lake, Light Red No. 7 D&C Lake, Violet No. 2 External D&C, Yellow No. 5 FD&C Lake, mica beige, mica blackstar red, mica Bordeaux, mica bronze, mica carmine red, mica cinnamon, mica diamond cluster, mica diamond sparkle, mica fine silver, mica gold, mica gold rush, mica interference copper, mica interference green, mica interference violet, mica light blue, mica luster black, mica magenta, mica majectic green, mica patina silver, mica pearl white, mica pink camel, mica purple sky, mica red, mica sand gold, mica sappan red, mica sugar blush, and mica walnut brown.

23. The method of 1, wherein the topical anti-acne composition further comprises an exfoliant comprising at least one of glycolic acid, lactic acid, citric acid, tartaric acid, salicylic acid, avocado powder, bacillus ferment, bamboo stem powder, pumpkin puree, glucosamine HCl, Laminario digitata extract, Saccharomyces cerevisiae extract, urea, walnut shell powder, and yogurt filtrate.

24. The method of 1, wherein the topical anti-acne composition further comprises an exfoliant present in 1-2 wt. %.

25. The method of 1, wherein the topical anti-acne composition has a pH of 4.0-7.0.

26. The method of 1, wherein the topical anti-acne composition has a pH of 4.5-6.0.
